(19) United States
(12) Patent Application Publication
(10) Pub. No.: US 2025/0002908 A1
(43) Pub. Date: Jan. 2, 2025

(54) COMPOSITIONS AND METHODS FOR INHIBITING COMPLEMENT COMPONENT 3 EXPRESSION

(71) Applicant: AstraZeneca Ireland Limited, Dublin 15 (IE)

(72) Inventors: Melissa Lasaro, Sandy Hook, CT (US); Susan Faas McKnight, Old Lyme, CT (US); Henryk T. Dudek, Belmont, MA (US); Jihye Park, Cambridge, MA (US); Bob Dale Brown, Littleton, MA (US); Chengjung Lai, Hudson, NH (US); SungKwon Kim, Cheshire, CT (US)

(73) Assignee: AstraZeneca Ireland Limited, Dublin 15 (IE)

(21) Appl. No.: 18/287,260

(22) PCT Filed: Apr. 20, 2022
(86) PCT No.: PCT/US2022/025648

§ 371 Date: Oct. 17, 2023

Related U.S. Application Data
(60) Provisional application No. 63/177,254, filed on Apr. 20, 2021.

(51) Int. Cl. C12N 15/113; A61P 13/12; A61P 19/02; A61P 25/28; A61P 37/06

(52) U.S. Cl. CPC C12N 15/113; A61P 13/12; A61P 19/02; A61P 25/28; A61P 37/06; C12N 2310/14; C12N 2310/315; C12N 2310/321; C12N 2310/344; C12N 2310/351; C12N 2310/3533; C12N 2310/531

(57) ABSTRACT

Described herein are oligonucleotides (e.g., RNAi oligonucleotides) containing a sense and antisense strands for targeting complement component 3 (C3) mRNA. The RNAi oligonucleotide may be used to inhibit C3 expression, levels, and/or activity in a cell. Also, described herein are methods for using an oligonucleotide (e.g., an RNAi oligonucleotide) for the prophylaxis or treatment of a disease, disorder, or condition mediated by complement pathway activation or dysregulation.

### SEQUENCE LISTING

The present application is being filed along with a Sequence Listing in electronic format. The Sequence Listing is provided as a file entitled 50694-093WO3_Sequence_Listing_4_18_22_ST25_FINAL created on Apr. 20, 2021, which is 77,827 bytes in size. The information in electronic format of the sequence listing is incorporated herein by reference in its entirety.

### BACKGROUND

The complement system plays a central role in the clearance of immune complexes and in immune responses to infectious agents, foreign antigens, virus-infected cells, and tumor cells. Complement consists of a group of more than 50 proteins that form part of the innate immune system. The complement system is poised to defend the body from microbial infections and functions to maintain tissue hemostasis. Complement is a tightly regulated enzymatic cascade that can be activated by one of three pathways: the classical pathway, in which antibody complexes trigger activation, the alternative pathway, which is constitutively activated at a low level by a process called “tickover”, and which can be amplified by bacterial pathogens or injured tissue surfaces, or the lectin pathway, which is initiated by mannose residues found on certain microorganisms including certain bacteria, fungi, and viruses. Uncontrolled activation or insufficient regulation of the complement pathway can lead to systemic inflammation, cellular injury, and tissue damage. Thus, the complement pathway has been implicated in the pathogenesis of a number of diverse diseases. Inhibition or modulation of complement pathway activity has been recognized as a promising therapeutic strategy. The number of treatment options available for these diseases are limited. Thus, developing innovative strategies to treat diseases associated with complement pathway activation or dysregulation is a significant unmet need.

Regardless of which complement pathway starts the process, complement activation converges at complement component 3 (C3) in the cascade. The C3 protein is central to driving several critical biologic processes including complement activation, opsonization and removal of pathogens, immune complexes and damaged cells, and regulation of humoral immunity and T cell adaptive immune responses.

C3 is an integral protein in the complement system that helps to initiate the complement pathway cascade. C3 activation through the classical pathway, alternative pathway, or lectin pathway results in the cleavage of C3 into the split products C3a and C3b. C3a is a potent anaphylatoxin and chemoattractant for neutrophils, eosinophils, and mast cells. C3b participates in the formation of the C3 convertase in the alternative pathway and C5 convertases in all three complement pathways, which in turn catapults the complement cascade into further activation of downstream terminal complement. C5 cleavage results in the formation of C5a, also a potent chemotactic driver and anaphylatoxin, and C5b, which rapidly assembles with complement proteins C6, 7, 8, and 9 into the pore-forming complex C5b-9 on pathogen or tissue surfaces. As a result, C3 may be an ideal target for inhibition or silencing in order to selectively inhibit the complement pathway as a method for treating diseases associated with complement pathway activation or dysregulation.

### SUMMARY OF THE DISCLOSURE

Described herein are oligonucleotides (e.g., RNAi oligonucleotides, including sense and antisense strand oligonucleotides) that target complement component (C3), which is known to play a role in complement pathway activation. The RNAi oligonucleotides, or a pharmaceutically acceptable salt thereof (e.g., a sodium salt thereof), may be used to treat patients with diseases associated with complement pathway activation or dysregulation.

In an aspect, the disclosure provides an RNAi oligonucleotide, or a pharmaceutically acceptable salt thereof, for reducing complement component 3 (C3) expression including a sense strand and an antisense strand, in which the sense strand and the antisense strand form a duplex region. The antisense strand includes a region of complementarity to a C3 mRNA target sequence of SEQ ID NO: 13 or 14, and the region of complementarity is at least 15 contiguous nucleotides in length. In some embodiments, the sense strand is 15 to 50 nucleotides in length (e.g., 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 nucleotides in length). In some embodiments, the sense strand is 18 to 36 nucleotides in length (e.g., 18, 19, 20, 21, 22, 23, 24, 25, 26, 27, 28, 29, 30, 31, 32, 33, 34, 35, and 36 nucleotides in length). In some embodiments, the antisense strand is 15 to 30 nucleotides in length (e.g., 15, 16, 17, 18, 19, 20, 21, 22, 23, 24, 25, 26, 27, 28, 29, and 30 nucleotides in length). In some embodiments, the antisense strand is 22 nucleotides in length and the antisense strand and the sense strand form a duplex region of at least 19 nucleotides in length, optionally at least 20 nucleotides in length. In some embodiments, the sense strand is 36 nucleotides in length and the antisense strand and the sense strand form a duplex region of at least 19 nucleotides in length, optionally at least 20 nucleotides in length. In some embodiments, the region of complementarity is at least 19 contiguous nucleotides in length, optionally at least 20 nucleotides in length.

In some embodiments, the 3′ end of the sense strand includes a stem-loop set forth as S1-L-S2, in which S1 is complementary to S2, and in which L forms a loop between S1 and S2 of 3-5 nucleotides in length. In some embodiments, L is a triloop or a tetraloop. In some embodiments, L is a tetraloop. In some embodiments, the tetraloop includes the nucleic acid sequence of SEQ ID NO: 8. In some embodiments, the S1 and S2 are 1-10 nucleotides in length, in which, optionally, S1 and S2 have the same length. In some embodiments, S1 and S2 are 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in length. In some embodiments, S1 and S2 are 6 nucleotides in length. In some embodiments, the stem loop region includes a nucleic acid sequence having at least 85% identity to SEQ ID NO: 7. In some embodiments, the stem loop region includes a nucleic acid sequence having at least 95% identity to SEQ ID NO: 7 (e.g., at least 95%, 96%, 97%, 98%, 99%, and 100% identity to SEQ ID NO: 7). In some embodiments, the stem loop region includes SEQ ID NO:7. In some embodiments, the stem-loop includes a nucleic acid having up to 1, 2, or 3 substitutions, insertions, or deletions relative to SEQ ID NO: 7.

In some embodiments, the antisense strand includes a 3′ overhang sequence of one or more nucleotides in length. In some embodiments, the antisense strand includes a 3′ overhang of at least 2 linked nucleotides. In some embodiments, the 3′ overhang sequence is 2 nucleotides in length, wherein optionally the 3′ overhang sequence is GG.

In some embodiments, the sense strand includes a nucleotide sequence of SEQ ID NO: 1 or SEQ ID NO: 4. In some embodiments, the antisense strand includes a nucleotide sequence of SEQ ID NO: 3 or SEQ ID NO: 6. In some embodiments, the sense strand and antisense strands include nucleotide sequences selected from the group consisting of (a) SEQ ID NOs: 1 and 3, respectively, and (b) SEQ ID NOS: 4 and 6, respectively. In some embodiments, the sense strand includes a nucleotide sequence as set forth in SEQ ID NO: 1 and the antisense strand includes a nucleotide sequence as set forth in SEQ ID NO: 3. In some embodiments, the sense strand includes a nucleotide sequence as set forth in SEQ ID NO: 4 and the antisense strand includes a nucleotide sequence as set forth in SEQ ID NO: 6. In some embodiments, the sense strand includes a nucleotide sequence as set forth in SEQ ID NO: 37 and the antisense strand includes a nucleotide sequence as set forth in SEQ ID NO: 38, as shown in Compound A. In some embodiments, the sense strand includes a nucleotide sequence as set forth in SEQ ID NO: 39 and the antisense strand includes a nucleotide sequence as set forth in SEQ ID NO: 40, as shown in Compound B. In some embodiments, the sense strand includes a nucleotide sequence as set forth in SEQ ID NO: 41 and the antisense strand includes a nucleotide sequence as set forth in SEQ ID NO: 42, as shown in Compound C. In some embodiments, the sense strand includes a nucleotide sequence as set forth in SEQ ID NO: 43 and the antisense strand includes a nucleotide sequence as set forth in SEQ ID NO: 44, as shown in Compound D. In some embodiments, the sense strand includes a nucleotide sequence as set forth in SEQ ID NO: 45 and the antisense strand includes a nucleotide sequence as set forth in SEQ ID NO: 46, as shown in Compound E. In some embodiments, the sense strand includes a nucleotide sequence as set forth in SEQ ID NO: 47 and the antisense strand includes a nucleotide sequence as set forth in SEQ ID NO: 48, as shown in Compound F. In some embodiments, the sense strand includes a nucleotide sequence as set forth in SEQ ID NO: 49 and the antisense strand includes a nucleotide sequence as set forth in SEQ ID NO: 50, as shown in Compound G. In some embodiments, the sense strand includes a nucleotide sequence as set forth in SEQ ID NO: 51 and the antisense strand includes a nucleotide sequence as set forth in SEQ ID NO: 52, as shown in Compound H. In some embodiments, the sense strand includes a nucleotide sequence as set forth in SEQ ID NO: 53 and the antisense strand includes a nucleotide sequence as set forth in SEQ ID NO: 54, as shown in Compound I.

Provided herein is an RNAi oligonucleotide, or a pharmaceutically acceptable salt thereof, including a sense strand and an antisense strand, in which the sense strand has a nucleic acid sequence with at least 85% (e.g., at least 86%, 87%, 88%, 89%, 90%, 91%, 92%, 93%, 94%, 95%, 96%, 97%, 98%, 99%, or 100%) sequence identity to either SEQ ID NO: 1 or SEQ ID NO: 4, and the antisense strand has a nucleic acid sequence with at least 85% (e.g., at least 86%, 87%, 88%, 89%, 90%, 91%, 92%, 93%, 94%, 95%, 96%, 97%, 98%, 99%, or 100%) sequence identity to either SEQ ID NO: 3 or SEQ ID NO: 6. In some embodiments, the sense strand has at least 95% (e.g., at least 96%, 97%, 98%, or 99%) sequence identity to either SEQ ID NO: 1 or SEQ ID NO: 4 and the antisense strand has at least 95% (e.g., at least 96%, 97%, 98%, or 99%) sequence identity to at least one of SEQ ID NO: 3 or SEQ ID NO: 6. In some embodiments, the sense strand has the nucleic acid sequence of SEQ ID NO: 1 or SEQ ID NO: 4, and the antisense strand has the nucleic acid sequence of SEQ ID NO: 3 or SEQ ID NO: 6. In some embodiments, the RNAi oligonucleotide, or pharmaceutically acceptable salt thereof, includes SEQ ID NO: 4 and SEQ ID NO: 6. In other embodiments, the RNAi oligonucleotide, or pharmaceutically acceptable salt thereof, includes SEQ ID NO: 1 and SEQ ID NO: 3.

In some embodiments, the antisense strand has at least 85% (e.g., at least 86%, 87%, 88%, 89%, 90%, 91%, 92%, 93%, 94%, 95%, 96%, 97%, 98%, 99%, or 100%) sequence identity to either SEQ ID NO: 3 or SEQ ID NO: 6.

In an embodiment, the sense strand includes a stem loop region that is not complementary to the antisense strand and a duplex region that is substantially complementary to the antisense strand. In another embodiment, the duplex region includes between 20 and 22 nucleosides in length.

In other embodiments, the stem loop region includes a nucleic acid sequence having at least 85% (e.g., at least 86%, 87%, 88%, 89%, 90%, 91%, 92%, 93%, 94%, 95%, 96%, 97%, 98%, and 99%) identity to SEQ ID NO: 7. In some embodiments, the stem loop region includes a nucleic acid sequence having at least 95% (e.g., at least 96%, 97%, 98%, and 99%) identity to SEQ ID NO: 7. In some embodiments, the stem loop region includes SEQ ID NO:7.

In some embodiments, the 4′-carbon of the sugar of the 5′-nucleotide of the antisense strand includes a phosphate analog. In some embodiments, the RNAi oligonucleotide, or pharmaceutically acceptable salt thereof, includes a uridine at the first position of the 5′ end of the antisense strand. In some embodiments, the uridine includes a phosphate analog. In some embodiments, the phosphate analog is 4′-O-monomethyl phosphonate. In some embodiments, the uridine including the phosphate analog includes the following structure:

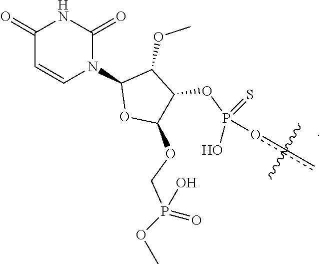

In some embodiments, the oligonucleotide includes at least one (e.g., at least 2, 5, 10, 15, 20, 30, and 40) modified nucleotide. In some embodiments, the oligonucleotide includes between 20 and 50 modified nucleotides (e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 modified oligonucleotides). In some embodiments, the oligonucleotide includes between 20 and 40 (e.g., between 25 and 40, 30 and 40, 35 and 40, 30 and 35, 25 and 35, 20 and 25, 21 and 30, and 31 and 40) modified nucleotides. In some embodiments, all of the nucleotides of the oligonucleotide are modified. In some embodiments, at least one (e.g., at least 2, 5, 10, 15, 20, 30, and 40) modified nucleotide includes a 2′-modification. In some embodiments, the 2′-modification is a 2′-fluoro or 2′-O-methyl, in which, optionally, the 2′-fluoro modification is 2′-fluoro deoxyribonucleoside and/or the 2′-O-methyl modification is 2′-O-methyl ribonucleoside.

In an embodiment, the RNAi oligonucleotide, or pharmaceutically acceptable salt thereof, includes between 40 and 50 (e.g., 41, 42, 43, 44, 45, 46, 47, 48, 49, and 50) 2′-O-methyl modifications, in which, optionally, the RNAi oligonucleotide, or pharmaceutically acceptable salt thereof, includes between 40 and 50 (e.g., 41, 42, 43, 44, 45, 46, 47, 48, 49, and 50) 2′-O-methyl ribonucleosides. In an embodiment, at least one (e.g., at least 2, at least 5, at least 10, at least 20, and at least 30) of nucleotides 1-7, 11-27, and 31-36 of the sense strand and one or more, or all, of nucleotides 1, 6, 8, 9, 11-13, and 15-22 of the antisense strand are modified with a 2′-O-methyl, such as a 2′-O-methyl ribonucleoside. In one embodiment, between 10 and 30 (e.g., between 12 and 28, 12 and 24, 12 and 20, 12 and 16, 16 and 30, 20 and 30, and 24 and 30) of nucleotides 1-7, 11-27, and 31-36 of the sense strand and one or more, or all, of nucleotides 1, 6, 8, 9, 11-13, and 15-22 of the antisense strand are modified with a 2′-O-methyl, such as a 2′-O-methyl ribonucleoside. In one embodiment, all of nucleotides 1-7, 12-27, and 31-36 of the sense strand and one or more, or all, of nucleotides 1, 6, 8, 9, 11-13, and 15-22 of the antisense strand are modified with a 2′-O-methyl, such as a 2′-O-methyl ribonucleoside. In some embodiments, all of nucleotides 1, 2, 4-7, 11, 14-16, 18-27, and 31-36 of the sense strand and one or more, or all, of nucleotides 1, 6, 9, 11, 13, 15, 17, 18, and 20-22 of the antisense strand are modified with a 2′-O-methyl, such as a 2′-O-methyl ribonucleoside.

In another embodiment, the RNAi oligonucleotide, or pharmaceutically acceptable salt thereof, includes between 5 and 15 (e.g., 6, 7, 8, 9, 10, 11, 12, 13, 14 and 15) 2′-fluoro modification, such as 2′-fluoro deoxyribonucleosides. In some embodiments, at least one (e.g., at least 2, 3, 4, 5, 6, or 7) of nucleotides 3, 8, 9, 10, 11, 12, 13, and 17 of the sense strand and one or more, or all, of nucleotides 2, 3, 4, 5, 7, 8, 10, 12, 14, 16, and 19 of the antisense strand are modified with a 2′-fluoro, such as 2′-fluoro deoxyribonucleoside. In another embodiment, between 2 and 4 of nucleotides 3, 8, 9, 10, 11, 12, 13, and 17 of the sense strand and one or more, or all, of nucleotides 2, 3, 4, 5, 7, 8, 10, 12, 14, 16, and 19 of the antisense strand are modified with a 2′-fluoro, such as 2′-fluoro deoxyribonucleoside. In another embodiment, all of nucleotides 8, 9, 10, and 11 of the sense strand and one or more, or all, of nucleotides 2, 3, 4, 5, 7, 10 and 14 of the antisense strand are modified with a 2′-fluoro, such as 2′-fluoro deoxyribonucleoside.

In some embodiments, the RNAi oligonucleotide, or pharmaceutically acceptable salt thereof, includes at least one (e.g., at least 2, at least 5, at least 10, at least 20, and at least 30) modified internucleotide linkage. In some embodiments, the at least one modified internucleotide linkage is a phosphorothioate linkage. In some embodiments, the RNAi oligonucleotide, or pharmaceutically acceptable salt thereof, has a phosphorothioate linkage between nucleotides 1 and 2 of the sense strand and nucleotides 1 and 2, 2 and 3, 20 and 21, and 21 and 22 of the antisense strand.

In one embodiment, there is no internucleotide linkage between the sense strand and the antisense strand.

In some embodiments, at least one nucleotide of the oligonucleotide is conjugated to one or more targeting ligands. In some embodiments, each targeting ligand includes a carbohydrate, amino sugar, cholesterol, polypeptide, or lipid. In some embodiments, each targeting ligand includes a N-acetylgalactosamine (GalNAc) moiety. In some embodiments, the GalNAc moiety is a monovalent GalNAc moiety, a bivalent GalNAc moiety, a trivalent GalNAc moiety or a tetravalent GalNAc moiety. In some embodiments, the RNAi oligonucleotide, or pharmaceutically acceptable salt thereof, includes between one and five 2′-O—N-acetylgalactosamine (GalNAc) moieties conjugated to the sense strand. In some embodiments, up to 4 nucleotides of L of the stem-loop are conjugated to a monovalent GalNAc moiety. In some embodiments, the RNAi oligonucleotide, or pharmaceutically acceptable salt thereof, includes between one and five (e.g., 2, 3, 4, and 5) GalNAc moieties conjugated to the sense strand. In some embodiments, at least one (e.g., at least 2, or at least 3) GalNAc moiety is conjugated to the loop region of the sense strand (SEQ ID NO: 8). In some embodiments, one or more of the nucleotides at nucleotides positions 28-30 on the sense strand is conjugated to a monovalent GalNAc moiety. In some embodiments, each of the nucleotides at positions 28-30 on the sense strand is conjugated to a monovalent GalNAc moiety.

In some embodiments, the nucleotides at positions 28-30 on the sense strand include the structure:

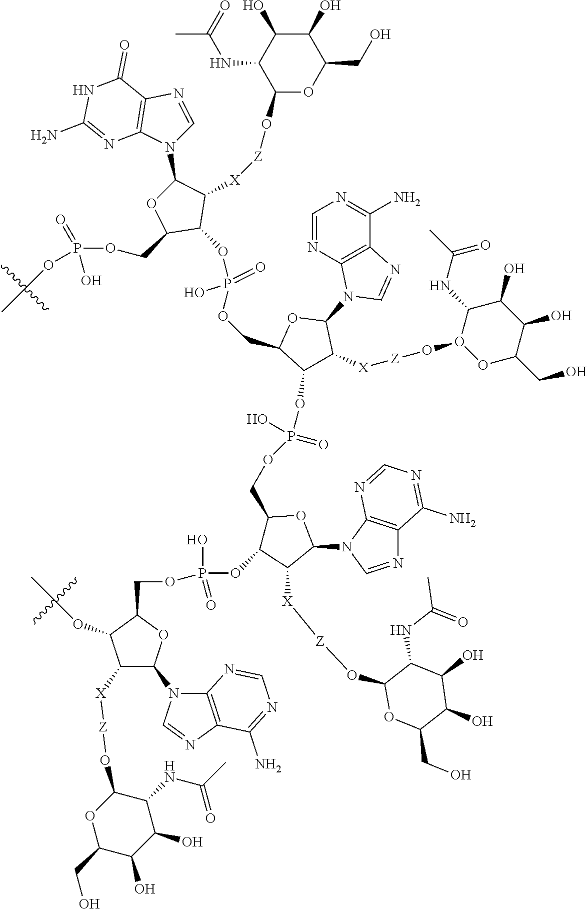

Z represents a bond, click chemistry handle, or a linker of 1 to 20, inclusive, consecutive, covalently bonded atoms in length, selected from the group consisting of substituted and unsubstituted alkylene, substituted and unsubstituted alkenylene, substituted and unsubstituted alkynylene, substituted and unsubstituted heteroalkylene, substituted and unsubstituted heteroalkenylene, substituted and unsubstituted heteroalkynylene, and combinations thereof; and X is an O, S, or N. In some embodiments, Z is an acetal linker. In some embodiments, X is O. In some embodiments, the nucleotides at positions 28-30 on the sense strand include the structure:

In some embodiments, the nucleotides at positions 28-30 on the sense strand include the structure:

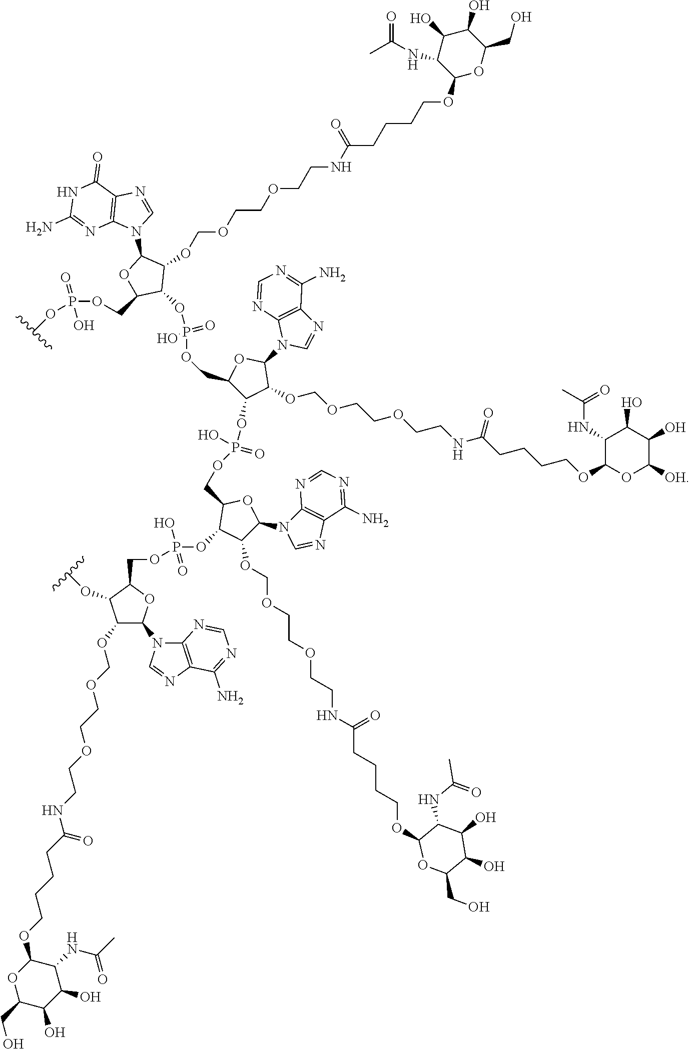 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](O)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

In one embodiment, the antisense strand is 13 to 27 (e.g., 13 to 25, 13 to 22, 13 to 20, 13 to 18, 13 to 15, 15 to 27, 18 to 27, 20 to 27, 22 to 27, and 25 to 27) nucleotides in length. In one embodiment, the antisense strand is 22 nucleotides in length.

In another embodiment, the sense strand is 20 to 50 (e.g., 22 to 50, 25 to 50, 30 to 50, 35 to 50, 40 to 50, 45 to 50, 20 to 45, 20 to 40, 20 to 35, 20 to 30, 20 to 25, 20 to 22) nucleotides in length. In one embodiment, the sense strand is 30 to 40 (e.g., 31, 32, 33, 34, 35, 36, 37, 38, 39, and 40) nucleotides in length.

In some embodiments, the sense strand forms a duplex with the antisense strand. In some embodiments, the duplex structure includes a duplex between all or a portion of the sense strand and all or a portion of the antisense strand. In some embodiments, the region of complementarity is 20 to 30 (e.g., 21, 22, 23, 24, 25, 26, 27, 28, 29 and 30) nucleotides in length. In some embodiments, the antisense strand and/or the sense strand includes a 3′ overhang of at least 2 (e.g., at least 3, at least 4, or at least 5) linked nucleotides. In some embodiments, the RNAi oligonucleotide, or pharmaceutically acceptable salt thereof, is a double-stranded ribonucleic acid (dsRNA). In some embodiments, the RNAi oligonucleotide, or pharmaceutically acceptable salt thereof, is a single-stranded ribonucleic acid.

In some embodiments, the RNA oligonucleotide includes a pharmaceutically acceptable salt. In some embodiments, the pharmaceutically acceptable salt is a sodium salt.

In another aspect, the disclosure provides a pharmaceutical composition including any one of the oligonucleotides (e.g., any of the RNAi oligonucleotides, or pharmaceutically acceptable salts thereof) described herein and a pharmaceutically acceptable carrier, excipient, or diluent.

In another aspect, the disclosure provides a vector encoding at least one strand of any one of the RNAi oligonucleotides, or pharmaceutically acceptable salts thereof, described herein.

In another aspect, the disclosure provides a vector encoding at least one strand of any one of the RNAi oligonucleotides, or pharmaceutically acceptable salts thereof, described herein having the DNA sequence of any one of SEQ ID NOs: 33-36.

In another aspect, the disclosure provides a cell including the vector described herein or any one of the RNAi oligonucleotides, or pharmaceutically acceptable salts thereof, described herein.

In another aspect, the disclosure provides a cell including the vector described herein or any of the oligonucleotides (e.g., any of the RNAi oligonucleotides, or pharmaceutically acceptable salts thereof) described herein.

In another aspect, the disclosure provides a method of treating a disease mediated by complement pathway activation or dysregulation, including contacting a cell of a subject with any of the oligonucleotides (e.g., RNAi oligonucleotides) described herein, the pharmaceutical composition described herein, the vector described herein, or the cell described herein. In some embodiments, the cell is contacted for a time sufficient to obtain degradation of an mRNA transcript of C3. In some embodiments, the expression of C3 in the cell is reduced. In some embodiments, the transcription of C3 in the cell is reduced. In some embodiments, the level and/or activity of C3 in the cell is reduced. In some embodiments, the level and/or activity of C3 is reduced by 10% to 100% (e.g., reduced by 10% to 90%, 10% to 80%, 10% to 70%, 10% to 60%, 10% to 50%, 10% to 40%, 10% to 30%, 10% to 20%, 20% to 100%, 30% to 100%, 40% to 100%, 50% to 100%, 60% to 100%, 70% to 100%, 80% to 100%, and 90% to 100%) relative to the level and/or activity of C3 in the cell of a subject that is not administered any one of the RNAi oligonucleotides, or pharmaceutically acceptable salts thereof, pharmaceutical composition, vector, or cell described herein. In some embodiments, the level and/or activity of C3 is reduced by 50% to 99% (e.g., 50% to 90%, 50% to 80%, 50% to 70%, 50% to 60%, 60% to 99%, 70% to 99%, 80% to 99%, and 90% to 99%) relative to the level and/or activity of C3 in the cell of a subject that is not administered any one of the RNAi oligonucleotides, or pharmaceutically acceptable salts thereof, pharmaceutical composition, vector, or cell described herein. In some embodiments, the subject is a mammal. In some embodiments, the subject is a human.

In another aspect, the disclosure provides a method for reducing C3 expression in a cell, a population of cells, or a subject, the method including the step of i) contacting the cell or the population of cells with any one of the RNAi oligonucleotides, or pharmaceutically acceptable salts thereof, pharmaceutical composition, or vector described herein; or ii) administering to the subject any one of the RNAi oligonucleotides, or pharmaceutically acceptable salts thereof, described herein, pharmaceutical composition, or vector described herein. In some embodiments, reducing C3 expression includes reducing an amount or level of C3 mRNA, an amount or level of C3 protein, or both. In some embodiments, the level of C3 mRNA, level of C3 protein, or both is reduced by 10% to 100% (e.g., reduced by 10% to 90%, 10% to 80%, 10% to 70%, 10% to 60%, 10% to 50%, 10% to 40%, 10% to 30%, 10% to 20%, 20% to 100%, 30% to 100%, 40% to 100%, 50% to 100%, 60% to 100%, 70% to 100%, 80% to 100%, and 90% to 100%) relative to the level of C3 mRNA, level of C3 protein, or both in the cell of a subject that is not administered any one of the RNAi oligonucleotides, or pharmaceutically acceptable salts thereof, pharmaceutical composition, vector, or cell described herein.

In some embodiments, the level of C3 mRNA, level of C3 protein, or both is reduced by 50% to 99% (e.g., 50% to 90%, 50% to 80%, 50% to 70%, 50% to 60%, 60% to 99%, 70% to 99%, 80% to 99%, and 90% to 99%) relative to the level of C3 mRNA, level of C3 protein, or both in the cell of a subject that is not administered any one of the RNAi oligonucleotides, or pharmaceutically acceptable salts thereof, pharmaceutical composition, vector, or cell described herein.

In some embodiments, the subject is identified as having a disease mediated by or associated with complement pathway activation or dysregulation (e.g., dysregulation of the alternative complement pathway, the classical complement pathway, and/or the lectin pathway. In some embodiments, the disease mediated by or associated with complement pathway activation or dysregulation is paroxysmal nocturnal hemoglobinuria (PNH), atypical hemolytic uremic syndrome (aHUS), IgA nephropathy, lupus nephritis, C3 glomerulopathy (C3G), dermatomyositis/autoimmune myositis, systemic sclerosis, demyelinating polyneuropathy, pemphigus, membranous nephropathy, focal segmental glomerular sclerosis (FSGS), bullous pemphigoid, epidermolysis bullosa acquisita (EBA), mucus membrane pemphigoid, ANCA vasculitis, hypocomplementemic urticarial vasculitis, immune complex small vessel vasculitis, cutaneous small vessel vasculitis, autoimmune necrotizing myopathy, rejection of a transplanted organ, such as kidney, liver, heart or lung transplant rejection, including antibody mediated rejection (AMR), such as chronic AMR (cAMR), antiphospholipid (aPL) Ab syndrome, glomerulonephritis, asthma, dense deposit disease (DDD), age related macular degeneration (AMD), systemic lupus erythematosus (SLE), rheumatoid arthritis (RA), severe refractory RA, felty syndrome, multiple sclerosis (MS), traumatic brain injury (TBI), spinal cord injury, ischemia reperfusion injury, preeclampsia, delayed graft function in acute kidney injury (DGF-AKI), cardiopulmonary bypass-associated acute kidney injury, hypoxic-ischemic encephalopathy, dialysis-induced thrombosis, Takayasu arteritis, relapsing polychondritis, acute/prophylactic graft vs. host disease, chronic graft vs. host disease, beta thalassemia, stem cell transplant-associated thrombotic microangiopathy, biliary atresia, inflammatory liver disease, Behcet's disease, ischemic stroke, intracerebral hemorrhage, scleroderma, scleroderma renal crisis, scleroderma-associated interstitial lung disease (SSC-ILD), sickle cell disease, autosomal dominant polycystic kidney disease (ADPKD), chemotherapy-induced peripheral neuropathy (CIPN), diabetic neuropathy, amyotrophic lateral sclerosis (ALS), diabetic nephropathy, diabetic retinopathy, geographic atrophy, pulmonary arterial hypertension, refractory severe asthma, chronic obstructive pulmonary disease, idiopathic pulmonary fibrosis (IPF), chronic lung allograft dysfunction, pulmonary morbidities in cystic fibrosis, hidradenitis suppurativa, nonalcoholic fatty liver disease (NASH), ankylosing spondylitis, hematopoietic stem cell transplantation-associated thrombotic microangiopathy (HSCT-TMA) (prevention), coronary artery disease, atherosclerosis, osteoporosis (prevention), osteoarthritis, high risk drusen, inflammatory bowel disease, ulcerative colitis, interstitial cystitis, dialysis induced complement activation, pyoderma gangrenosum, chronic heart failure, autoimmune myocarditis, nasal polyposis, acute and chronic pancreatitis, atherosclerosis, eosinophilic esophagitis, eosinophilic granulomatosis, hypereosinohilic syndrome, wound healing, and thrombotic thrombocytopenia purpura (TTP). In some embodiments, the subject is identified as having antibody mediated rejection (AMR), such as chronic AMR.

In some embodiments, the disclosure provides a method of treating antibody mediated rejection (AMR), such as chronic AMR (CAMR), including contacting a cell of a subject with any one of the RNAi oligonucleotides, or pharmaceutically acceptable salts thereof, pharmaceutical composition, vector, or cell described herein. In some embodiments, any one of the RNAi oligonucleotides, or pharmaceutically acceptable salts thereof, pharmaceutical composition, vector, or cell described herein is for use in the prophylaxis or treatment of antibody mediated rejection (AMR), such as chronic AMR (cAMR) in a subject in need thereof.

In some embodiments, the RNAi oligonucleotide, or pharmaceutically acceptable salt thereof, the pharmaceutical composition, the vector, or the cell is formulated for daily, weekly, monthly, or yearly administration. In some embodiments, the RNAi oligonucleotide, or pharmaceutically acceptable salt thereof, the pharmaceutical composition, the vector, or the cell is formulated for intravenous, subcutaneous, intramuscular, oral, nasal, sublingual, intrathecal, and intradermal administration. In some embodiments, the RNAi oligonucleotide, or pharmaceutically acceptable salt thereof, the pharmaceutical composition, the vector, or the cell is formulated for subcutaneous administration.

In one embodiment, the oligonucleotide (e.g., the RNAi oligonucleotide, or pharmaceutically acceptable salt thereof), or a composition thereof is formulated for daily, weekly, monthly, or yearly administration. In one embodiment, the oligonucleotide is formulated for subcutaneous, intravenous, intramuscular, oral, nasal, sublingual, intrathecal, and intradermal administration. In one embodiment, the oligonucleotide is formulated for subcutaneous administration. In one embodiment, the oligonucleotide is formulated for administration at a dosage of between about 0.1 mg/kg to about 150 mg/kg. (e.g., 0.1 mg/kg and 125 mg/kg, 0.1 mg/kg and 100 mg/kg, 0.1 mg/kg and 75 mg/kg, 0.1 mg/kg and 50 mg/kg, 0.1 mg/kg and 25 mg/kg, 0.1 mg/kg and 15 mg/kg, 0.1 mg/kg and 10 mg/kg, 0.1 mg/kg and 5 mg/kg, 5 mg/kg and 150 mg/kg, 25 mg/kg and 150 mg/kg, and 50 mg/kg and 150 mg/kg). In one embodiment, the oligonucleotide is formulated for administration at a dosage of between about 0.5 mg/kg to about 15 mg/kg (e.g., 0.5 mg/kg to 13 mg/kg, 0.5 mg/kg to 10 mg/kg, 0.5 mg/kg and 5 mg/kg, 0.5 mg/kg and 1 mg/kg, 1 mg/kg and 15 mg/kg, 5 mg/kg and 15 mg/kg, and 10 mg/kg and 15 mg/kg).

In some embodiments, the oligonucleotide is formulated for administration in combination with one or more additional therapeutic agents.

In another aspect, the disclosure provides a kit including an oligonucleotide (e.g., an RNAi oligonucleotide, or pharmaceutically acceptable salt thereof) described herein, a pharmaceutical composition described herein, a vector described herein, or a cell described herein.

In another aspect, the disclosure provides an oligonucleotide (e.g., an RNAi oligonucleotide, or pharmaceutically acceptable salt thereof) described herein, a pharmaceutical composition described herein, a vector described herein, or a cell described for use in the prevention or treatment of a disease mediated by or associated with complement pathway activation or dysregulation (e.g., activation or dysregulation of the alternative, classical, and/or lectin pathway).

In another aspect, the disclosure provides the oligonucleotide (e.g., the RNAi oligonucleotide, or pharmaceutically acceptable salt thereof), the pharmaceutical composition, the composition, the vector, or the cell, as described herein, in which the RNAi oligonucleotide, or pharmaceutically acceptable salts thereof, pharmaceutical composition, composition, vector, or cell is administered or is formulated for administration subcutaneously.

### BRIEF DESCRIPTION OF THE DRAWINGS

FIG. 1A shows the chemical structure of the antisense strand of Compound A.

FIG. 1B shows the chemical structure of the sense strand of Compound A.

FIG. 1C-1 and FIG. 1C-2 show the chemical structure of the RNAi oligonucleotide of Compound A.

FIG. 1D shows the nucleic acid sequence for the sense and antisense strands of Compound A.

FIG. 1E show a schematic drawing of the double stranded oligonucleotide of Compound A.

FIG. 2A-1 and FIG. 2A-2 show the chemical structure of the sense and antisense strands of Compound B.

FIG. 2B shows the nucleic acid sequence for the sense and antisense strands of Compound B.

FIG. 3A is a graph showing the results of an in vitro screen completed in HepG2 cells measuring the percent of C3 mRNA remaining after cells were treated with various oligonucleotides in an amount of 1 nM.

FIG. 3B is a graph showing the results of an in vitro screen completed in HepG2 cells measuring the percent of C3 mRNA remaining as a result of treating the cells with various oligonucleotides in an amount of 0.1 nM, and 1 nM.

FIG. 4A are schematic drawings of the RNAi oligonucleotide of Compounds A-I.

FIG. 4B is a graph showing the results of an in vivo screen of Compounds A, B, and C in CD-1 mice expressing human C3 cDNA after hydrodynamic injection. A RT-qPCR measurement of the percent human C3 mRNA in the liver four days after administration of a single, subcutaneous dose of 1 mg/kg of Compound A, B and C as compared to phosphate buffered saline (PBS) control group.

FIG. 4C is a graph showing the results of an in vivo screen of Compounds A, D, E, F, G, H, and I in CD-1 mice expressing human C3 cDNA after hydrodynamic injection. A RT-qPCR measurement of the percent human C3 mRNA in the liver four days after administration of a single, subcutaneous dose of 0.5 mg/kg of Compound A, D, E, F, G, H and I as compared to PBS control group.

FIG. 5 is a graph showing measurement of the percent C3 mRNA in the liver of cynomolgus macaques pre dose, 28 days and 56 days after treatment with a single dose of 4 mg/kg Compound A, Compound B, or any one of Compounds C—I as compared to PBS administered as a control.

FIG. 6A is a graph showing measurement of the percent of C3 mRNA in the liver of cynomolgus macaques after treatment with 1 mg/kg or 2 mg/kg Compound A or Compound B on days 0, 28, 56, and 84 as compared to PBS administered as a control.

FIG. 6B is a graph showing measurement of the percent of C3 in serum of cynomolgus macaques after treatment with 1 mg/kg or 2 mg/kg Compound A or Compound B as compared to PBS administered as a control.

FIG. 7 is a graph showing the approximate ED₅₀ for Compound A and Compound B measured as C3 mRNA in liver of cynomolgus macaques 28 days after a single dose of Compound A or Compound B at 2 mg/kg.

FIG. 8 is a graph showing the percent of complement activity (AP) in serum of cynomolgus macaques after treatment 2 mg/kg Compound A or Compound B on days 0, 28, 56 and 84 as measured by WIESLAB® ELISA-based functional assay. PBS was administered in the same multidose regimen as a control group.

FIG. 9 is a graph showing the percent of lysis from serum of cynomolgus macaques after treatment 1 mg/kg or 2 mg/kg Compound A on days 0, 28, 56 and 84 as measured by hemolysis of rabbit erythrocytes method. PBS was administered in the same multidose regimen as a control group.

FIG. 10A is a graph showing RT-qPCR measurement of the percent C3 mRNA in the liver of CD-1 mice after administration of a single, subcutaneous dose of 0.5 mg/kg, 1 mg/kg, and 6 mg/kg of Compound J as compared to PBS administered as a control. The levels of hepatic knockdown were followed for 70 days, and 5 mice were sacrificed at each time point for measurements.

FIG. 10B is a graph showing an ELISA assay measurement of the percent C3 circulating protein in serum of CD-1 mice over a 70-day period after being administered a single, subcutaneous dose of Compound J of 0.5 mg/kg, 1 mg/kg, and 6 mg/kg as compared to PBS administered as a control.

FIG. 11 is a graph showing the stem loop-qPCR measurement of the amount of siRNA exposure in the plasma, liver, kidney, and spleen tissue of CD-1 mice administered a single, subcutaneous dose of 6 mg/kg of Compound J over a period of 672 hours. Five mice were sacrificed at each time point for measurements.

FIG. 12A is a graph showing RT-qPCR measurement of the percent of C3 mRNA in the liver of CD-1 mice over a period of 70 days following administration of 4 doses of 1 mg/kg or 6 mg/kg Compound J on days 0, 14, 28, and 42 as compared to PBS administered as a control.

FIG. 12B is a graph showing an ELISA assay measurement of the C3 serum protein in CD-1 mice over a period of 70 days following administration of 4 doses of 1 mg/kg or 6 mg/kg Compound J on days 0, 14, 28, and 42. C3 levels were calculated as a percentage to C3 serum levels measured from PBS control group (n=5/timepoint).

FIG. 13A is a graph showing stem loop-qPCR measurement of the concentration of Compound J in liver tissue of CD-1 mice dosed with 4 doses of 1 mg/kg Compound J on Days 0, 14, 28, and 42.

FIG. 13B is a graph showing stem loop-qPCR measurement of the concentration of Compound J in plasma of CD-1 mice dosed with 4 doses of 1 mg/kg Compound J on Days 0, 14, 28, and 42.

FIG. 14 is a set of images showing in situ hybridization of fluorescent tags to C3 and properdin to monitor glomerular complement deposition in the kidney of NZB/W F1 mice treated with 0.5 mg/kg, 3 mg/kg, or 6 mg/kg of Compound J monthly for 18 weeks from the ages of 21 to 37 weeks in comparison to naïve C57BL/6 mice and age matched PBS-treated NZB/W F1 as controls.

FIG. 15A is a graph showing the percentage of C3 mRNA in liver of NZB/W F1 mice after receiving monthly subcutaneous doses of 0.5 mg/kg, 3 mg/kg, or 6 mg/kg of Compound J at 21 weeks of age and terminating at 29 weeks of age (n=10/timepoint).

FIG. 15B is a graph showing the percentage of C3 serum protein of NZB/W F1 mice after receiving monthly subcutaneous doses of 0.5 mg/kg, 3 mg/kg, or 6 mg/kg of Compound J at 21 weeks of age and terminating at 29 weeks of age (n=10/timepoint).

FIG. 16A is a graph showing the absorbance measured at 450 nm measuring circulating immune complexes by IgG capture in 29-week old NZB/W F1 mice who had been subcutaneously dosed starting at 21 weeks of age with 0.5 mg/kg, 3 mg/kg, or 6 mg/kg of Compound J.

FIG. 16B is a graph showing the absorbance measured at 450 nm measuring circulating immune complexes by C1q capture in 37-week old NZB/W F1 mice monthly dosed with 0.5 mg/kg, 3 mg/kg, or 6 mg/kg of Compound J starting at 21 weeks of age.

FIG. 17 is a set of images showing in situ hybridization of fluorescent tags to C3 and properdin to monitor complement deposition on the glomeruli of MRL/Ipr mice treated with subcutaneous doses of 6 mg/kg of Compound J every two weeks from the ages of 8 to 16 weeks compared to PBS control group.

FIG. 18 is a set of images showing in situ hybridization of fluorescent tags to C3 and properdin to monitor glomerular complement deposition in the kidney of CFH−/−mice treated with 0.5 mg/kg, 3 mg/kg or 6 mg/kg of Compound J monthly for 4 months from the ages of 4 to 8 months compared to PBS control group. Kidneys were collected and imaged 4 weeks after last dose.

FIG. 19 is a graph showing the RT-qPCR measurement of the percent of C3 mRNA in the liver of CFH—/-mice treated with 0.5 mg/kg, 3 mg/kg or 6 mg/kg of Compound J monthly for 4 months from the ages of 4 to 8 months as compared to CFH—/-mice administered with PBS as control group.

FIG. 20A is a graph showing the clinical score of the hind paws from collagen antibody-induced arthritis model in which arthritis was induced on Day 0 and an LPS booster on Day 3 and then prophylactically treated with 3 doses of 1 mg/kg or 6 mg/kg dose of Compound J on day-7, 0 and 7. PBS treated CAIA animals were used as control group.

FIG. 20B is a graph showing the clinical score of the hind paws from collagen antibody-induced arthritis model in which arthritis was induced on Day 0 and an LPS booster on Day 3 and then therapeutically treated with a single dose of 1 mg/kg or 6 mg/kg dose of Compound J on day 5 post disease induction. PBS-treated CAIA animals were used as control group.

FIG. 21A is a set of images of hind paw inflammation on Day 11 of a CAIA mouse model in which arthritis was induced with a collagen antibody administered on Day 0 and an LPS booster on Day 3 and then prophylactically treated with 3 doses of 6 mg/kg dose of Compound J on day-7, 0 and 7. PBS treated CAIA animals were used as control group.

FIG. 21B is a set of images of hind paw inflammation on Day 13 of a CAIA mouse model in which arthritis was induced with a collagen antibody administered on Day 0 and an LPS booster on Day 3 and then therapeutically treated with a single 6 mg/kg dose of Compound J on day 5 post disease induction. PBS treated CAIA animals were used as control group.

FIG. 22A is a set of images of H&E staining demonstrating the reduction of mononuclear cells infiltration to the hind paws after prophylactic treatment with 3 doses of 6 mg/kg dose of Compound J on day-7, 0 and 7. Naïve and PBS-treated CAIA animals were used as negative and positive controls for inflammation, respectively.

FIG. 22B is a set of images of H&E staining demonstrating the reduction of mononuclear cells infiltration to the hind paws after therapeutic treatment with a single 6 mg/kg dose of Compound J in a CAIA-induced arthritis mouse model 5 days post disease induction. Naïve and PBS treated CAIA animals were used as negative and positive controls for inflammation, respectively.

FIG. 23 is a set of images of Safranin O staining demonstrating prevention of cartilage erosion and pannus formation and H&E staining demonstrating reduction of mononuclear cell infiltration in the knee joint of CAIA-induced arthritis model after animals were prophylactically treated with 3 doses of 6 mg/kg Compound J on day-7, 0 and 7. Naïve and PBS treated CAIA animals were used as negative and positive controls, respectively.

FIG. 24A is a set of images of H&E staining demonstrating reduction of mononuclear cell infiltration in the knee joint of CAIA-induced arthritis model after animals were therapeutically treated with a single dose of 6 mg/kg Compound J on day 5 post disease induction. Naïve and PBS treated CAIA animals were used as negative and positive controls, respectively.

FIG. 24B is a set of images of Safranin O staining demonstrating prevention of cartilage erosion and pannus formation in the knee joint of CAIA-induced arthritis model after animals were therapeutically treated with a single dose of 6 mg/kg Compound J on day 5 post disease induction. Naïve and PBS treated CAIA animals were used as negative and positive controls, respectively.

FIG. 25 is a set of images of lymphocyte (CD45+) staining of the hind paws of CAIA-induced arthritis animals demonstrating the reduction of immune cell infiltration after therapeutic treatment with a single dose of 6 mg/kg of Compound J on day 5 post disease induction. Naïve and PBS treated CAIA animals were used as negative and positive controls, respectively.

FIG. 26 is a set of images of neutrophils and macrophages (CD11b+) staining of the hind paws of CAIA-induced arthritis animals demonstrating the reduction of immune cell infiltration after therapeutic treatment with a single dose of 6 mg/kg of Compound J on day 5 post disease induction. Naïve and PBS treated CAIA animals were used as negative and positive controls, respectively.

FIG. 27 is a set of images of macrophage (F4/80+) staining of the hind paws of CAIA-induced arthritis animals demonstrating the reduction of immune cell infiltration after therapeutic treatment with a single dose of 6 mg/kg of Compound J on day 5 post disease induction. Naïve and PBS treated CAIA animals were used as negative and positive controls, respectively.

FIG. 28 is a set of images of in situ hybridization of fluorescent tags to C3 mRNA (red) to monitor local complement expression and CD45+ cells (green-lymphocytes) infiltration to the hind paw of CAIA-induced animals after therapeutic treatment with a single 6 mg/kg dose of Compound J on day 5 post disease induction.

FIG. 29 is a graph showing the mean clinical score from 2 experiments using MOG-induced experimental autoimmune encephalomyelitis (EAE) mice in which disease was induced on Day 0 and receive 2 doses of Pertussis toxin on Day 0 and 1 and then therapeutically treated with 5 weekly doses of 6 mg/kg dose of Compound J starting on day 7 post disease induction. PBS treated EAE animals were used as disease positive control and C3-deficient (C3−/−) were used as negative control for C3 expression.

FIG. 30 is a set of representative images of Luxol fast blue spinal cord staining of MOG-induced EAE mice after 5 weekly doses of 6 mg/kg of Compound J in comparison to naïve, PBS-treated EAE mice (disease control), and C3-deficient mice MOG-induced EAE.

FIG. 31A is a graph showing the amount of liver C3 mRNA in MOG-induced EAE mice after 5 weekly doses of 6 mg/kg of Compound J in comparison to naïve, PBS-treated EAE mice (positive control), naïve C3 deficient mice (C3−/−) and C3 deficient mice MOG-induced EAE (negative control).

FIG. 31B is a graph showing the amount of serum C3 in MOG-induced EAE mice after 5 weekly doses of 6 mg/kg of Compound J in comparison to naïve, PBS-treated EAE mice (disease positive control), naïve C3 deficient mice (C3−/−) and C3-deficient mice MOG-induced EAE (negative control for C3 expression).

FIG. 32A is a graph showing the mean concentration of Compound A versus time (in hours) in plasma of cynomolgus macaques following administration of a single IV or SC dose of Compound A at 3 mg/kg.

FIG. 32B is a graph showing the mean concentration of Compound A versus time (in hours) in liver of cynomolgus macaques following administration of a single IV or SC dose of Compound A at 3 mg/kg.

FIG. 33 is a graph showing mean percent (+SD) C3 mRNA expression in liver of cynomolgus macaques following a single dose of Compound A at 3 mg/kg by SC or IV injection in comparison to saline (control).

FIG. 34 is a graph showing mean expression (+SD) C3 protein in serum of cynomolgus macaques following administration of a single IV or SC dose of Compound A at 3 mg/kg in comparison to saline (control).

FIG. 35 is a graph showing complement C3 classical pathway activity in cynomolgus macaques following administration of a single IV or SC dose of Compound A at 3 mg/kg in comparison to saline (control).

FIG. 36 is a graph showing complement C3 lectin pathway activity in cynomolgus macaques following administration of a single IV or SC dose of Compound A at 3 mg/kg in comparison to saline (control).

FIG. 37 is a graph showing complement C3 alternative pathway activity in cynomolgus macaques following administration of a single IV or SC dose of Compound A at 3 mg/kg in comparison to saline (control).

### DEFINITIONS

As used herein, the terms “about” and “approximately” refer to an amount that is ±10% of the recited value and is optionally ±5% of the recited value, or more optionally ±2% of the recited value.

As used herein, “administering” and “administration” refers to any method of providing a pharmaceutical preparation to a subject. The oligonucleotides described herein may be administered by any method known to those skilled in the art. Suitable methods for administering an oligonucleotide may include, for example, orally, by injection (e.g., intravenously, intraperitoneally, intramuscularly, intravitreally, and subcutaneously), drop infusion preparations, and the like. Methods of administering an oligonucleotide may include subcutaneous administration. Oligonucleotides prepared as described herein may be administered in various forms, depending on the disorder to be treated and the age, condition, and body weight of the subject, as is known in the art. A preparation can be administered prophylactically; that is, administered to decrease the likelihood of developing a disease or condition.

As used herein, an “agent that reduces the level and/or activity of C3” refers to any oligonucleotide (e.g., an RNAi oligonucleotide) disclosed herein that can be used (e.g., administered) to reduce the level or expression of C3 in a cell or subject, such as in the subject's cells or serum. By “reducing the level of C3,” “reducing expression of C3,” and “reducing transcription of C3” is meant decreasing the level, decreasing the expression, or decreasing the transcription of C3 mRNA and/or C3 protein in a cell or subject, e.g., by administering an RNAi oligonucleotide (such as those described herein) to the cell or subject. The level of C3 mRNA and/or C3 protein may be measured using any method known in the art (e.g., by measuring the level of C3 mRNA or level of C3 protein in a cell or a subject). The reduction may be a decrease in the level, expression, or transcription of C3 mRNA and/or C3 protein of about 5% or more (e.g., about 10%, 20%, 30%, 40%, 50%, 60%, 70%, 80%, 90%, 95%, 96%, 97%, 98%, 99%, or about 100%) in a cell or subject compared to prior to treatment or relative to a level of C3 mRNA or C3 protein in an untreated subject (e.g., a subject with a disease or disorder associated with complement activation or dysregulation (e.g., activation or dysregulation of C3) or relative to a control subject (e.g., a healthy subject (e.g., a subject without a disease or disorder associated with complement activation or dysregulation (e.g., activation or dysregulation of C3)). The C3 may be any C3 (such as, e.g., mouse C3, rat C3, monkey C3, or human C3), as well as variants or mutants of C3. Thus, the C3 may be a wild-type C3, a mutant C3, or a transgenic C3 in the context of a genetically manipulated cell, group of cells, or organism. “Reducing the activity of C3” also means decreasing the level of an activity related to C3 (e.g., by reducing the activation of the complement pathway associated with a disease mediated by complement pathway activation or dysregulation). The activity of C3 may decreased by about 5% or more (e.g., about 10%, 20%, 30%, 40%, 50%, 60%, 70%, 80%, 90%, 95%, 96%, 97%, 98%, 99%, or about 100%. The activity level of C3 may be measured using any method known in the art. The reduction may be a decrease in the level, expression, or transcription of C3 mRNA and/or C3 protein of at least about 5% or more (e.g., about 10%, 20%, 30%, 40%, 50%, 60%, 70%, 80%, 90%, 95%, 96%, 97%, 98%, 99%, or about 100% or more, relative to a cell or a subject not treated with an RNAi oligonucleotide disclosed herein). This reduction in the level, expression, or transcription of C3 mRNA and/or C3 protein may be for a period of at least one day or more (e.g., at least 2 days, 3 days, 4 days, 5 days, 10 days, 15 days, 20 days, 30 days, 40 days, 50 days, 60 days, 70 days, 80 days, 90 days, 100 days, 110 days, 120 days, or more). The reduction may be a decrease in the amount of C3 protein in blood of a treated subject (e.g., a human subject) of at least 75-175 mg/dL (e.g., 75-100 mg/dL, 75-125 mg/L, 75-150 mg/dL, 150 mg/dL-175 mg/dL, 125-175 mg/dL, and 100-175 mg/dL).

The term “alternative nucleoside” or “alternative nucleotide” refers to a nucleoside having an alternative sugar or an alternative nucleobase, such as those described herein. An alternative nucleoside may include a nucleoside in which the nucleobase moiety is modified by changing the purine or pyrimidine into a modified purine or pyrimidine, such as substituted purine or substituted pyrimidine, such as an “alternative nucleobase” selected from isocytosine, pseudoisocytosine, 5-methyl cytosine, 5-thiozolo-cytosine, 5-propynyl-cytosine, 5-propynyl-uridine, 5-bromouridine, 5-thiazolo-uridine, 2-thio-uridine, pseudouridine, 1-methylpseudouridine, 5-methoxyuridine, 2′-thio-thymine, inosine, diaminopurine, 6-aminopurine, 2-aminopurine, 2,6-diaminopurine, and 2-chloro-6-aminopurine. An alternative nucleoside may also include a nucleoside where the sugar moiety is modified; for example, 2′-O-methyladenosine, 2′-O-methylguanosine, 2′-O-methylcytosine, 2′-O-methyluridine, 2-fluoro-deoxyadenosine, 2-fluoro-deoxyguanosine, 2-fluoro-deoxycytidine, and 2-fluoro-deoxyuridine.

Exemplary nucleobases having an alternative uracil include pseudouridine (w), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s₂U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm⁵s²U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp³U), 1-methyl-3-(3-amino-3-carboxypropyl) pseudouridine (acp³ψ), 5-(isopentenylaminomethyl) uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino) uridine.

Exemplary nucleobases having an alternative cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4, N4,2′-O-trimethyl-cytidine (m⁴₂Cm), 1-thio-cytidine, 2′-F-ara-cytidine, 2′-F-cytidine, and 2′-OH-ara-cytidine.

Exemplary nucleobases having an alternative adenine include 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms²m⁶A), N6-isopentenyl-adenosine (i⁶A), 2-methylthio-N6-isopentenyl-adenosine (ms²i⁶A), N6-(cis-hydroxyisopentenyl) adenosine (io⁶A), 2-methylthio-N6-(cis-hydroxyisopentenyl) adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

Exemplary nucleobases having an alternative guanine include inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m²₂G), N2,7-dimethyl-guanosine (m^{2,7}G), N2,N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (m¹Gm), N2,7-dimethyl-2′-O-methyl-guanosine (m^{2,7}Gm), 2′-O-methyl-inosine (Im), 1,2′-O-dimethyl-inosine (m¹Im), 2′-O-ribosylguanosine (phosphate) (Gr(p)), 1-thio-guanosine, 06-methyl-guanosine, 2′-F-ara-guanosine, and 2′-F-guanosine.

The nucleobase moieties may be indicated by the letter code for each corresponding nucleobase, e.g., A, T, G, C, or U, wherein each letter may optionally include alternative nucleobases of equivalent function.

The term “antisense,” as used herein, refers to an oligonucleotide that is sufficiently complementary to all or a portion of a gene, primary transcript, or processed mRNA (e.g., the sequence of C3 (e.g., SEQ ID NO: 12)), so as to interfere with expression of the endogenous gene (e.g., C3).

The terms “antisense strand” and “guide strand” refer to the strand of an RNAi oligonucleotide (e.g., a dsRNA) that includes a region that is substantially complementary to a target sequence, e.g., a C3 mRNA (e.g., SEQ ID NO: 12).

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 10 nucleotides of a 21-nucleotide nucleic acid molecule” means that a range of from 10-21 nucleotides, such as, e.g., 10, 11, 12, 13, 14, 15, 16, 17, 18, 19, 20, or 21 nucleotides, have the indicated property. When “at least” is present before a series of numbers or a range, it is understood that “at least” can modify each of the numbers in the series or range.

As used herein, the term “attenuates” means reduces or effectively halts. As a non-limiting example, one or more of the treatments provided herein may reduce or effectively halt the onset or progression of a disease mediated by complement pathway activation or dysregulation (e.g., C3 activation or dysregulation) in a subject. This attenuation may be exemplified by, for example, a decrease in one or more aspects (e.g., symptoms, tissue characteristics, and cellular, inflammatory or immunological activity, etc.) of a disease associated with complement pathway activation or dysregulation, such as for example, one or more of the diseases associated with complement pathway activation or dysregulation disclosed herein.

The term “cDNA” refers to a nucleic acid sequence that is a DNA equivalent of an mRNA sequence (i.e., having uridine substituted with thymidine). Generally, the terms cDNA and mRNA may be used interchangeably in reference to a particular gene (e.g., C3 gene) as one of skill in the art would understand that a cDNA sequence is the same as the mRNA sequence with the exception that uridines are read as thymidines.

As used herein the terms “C3” and “complementary component 3” refers to the protein or gene encoding the complementary component 3. The term “C3” refers to natural variants of the wild-type C3 protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type human C3, which is set forth in NCBI Reference No: NP_000055.2 or in SEQ ID NO: 11. The term “C3” also refers to natural variants of the wild-type C3 polynucleotide, such as polynucleotides having at least 85% identity (e.g., 85%, 86%, 87%, 88%, 89%, 90%, 91%, 92%, 93%, 94%, 95%, 96%, 97%, 98%, 99%, 99.9% identity, or more) to the nucleic acid sequence of wild-type human C3 which is set forth in NCBI Reference No. NM_000064.4 or in SEQ ID NO: 12.

As used herein, a “combination therapy” or “administered in combination with” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be affected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the term “complement pathway activation or dysregulation” refers to any aberration in the ability of the complement pathway, including the classical pathway, alternative pathway, and lectin pathway, to provide host defense against pathogens and clear immune complexes and damaged cells and for immunoregulation. Complement pathway activation or dysregulation can occur in the fluid phase and at the cell surface and can lead to excessive complement activation or insufficient regulation, both causing tissue injury.

As used herein, “complementary,” when used to describe a first nucleotide or nucleoside sequence in relation to a second nucleotide or nucleoside sequence, refers to the ability of an oligonucleotide comprising the first nucleotide or nucleoside sequence to hybridize and form a duplex structure under certain conditions with an oligonucleotide comprising the second nucleotide sequence, as will be understood by a skilled person in the art. Such conditions can, for example, be stringent conditions, where stringent conditions can include: 400 mM NaCl, 40 mM PIPES pH 6.4, 1 mM EDTA, 50° C., or 70° C., for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides or nucleosides. “Complementary” sequences, as used herein, can also include, or be formed entirely from, non-Watson-Crick base pairs and/or base pairs formed from non-natural and alternative nucleotides or nucleosides, in so far as the above requirements with respect to their ability to hybridize are fulfilled. Such non-Watson-Crick base pairs include, but are not limited to, G: U Wobble or Hoogstein base pairing. Complementary sequences within an oligonucleotide (e.g., RNAi oligonucleotide), or between an oligonucleotide and a target sequence, as described herein, include base-pairing of the oligonucleotide comprising a first nucleotide or nucleoside sequence to an oligonucleotide comprising a second nucleotide or nucleoside sequence over the entire length of one or both nucleotide or nucleoside sequences. Such sequences can be referred to as “fully complementary” with respect to each other herein. Where a first sequence is referred to as “substantially complementary” with respect to a second sequence, the two sequences can be fully complementary or they can form one or more, but generally not more than 5, 4, 3, or 2, mismatched base pairs upon hybridization for a duplex of up to 30 base pairs, while retaining the ability to hybridize under the conditions most relevant to their ultimate application, e.g., reduction of expression via a RISC pathway. “Substantially complementary” can also refer to an oligonucleotide that is substantially complementary to a contiguous portion of the mRNA of interest (e.g., an mRNA encoding a C3). For example, an oligonucleotide is complementary to at least a part of a C3 mRNA if the sequence is substantially complementary to a non-interrupted portion of an mRNA encoding C3. However, where two oligonucleotides are designed to form, upon hybridization, one or more single stranded overhangs, such overhangs shall not be regarded as mismatches with regard to the determination of complementarity. For example, an oligonucleotide (e.g., RNAi oligonucleotide) comprising one oligonucleotide of 22 linked nucleosides in length and another oligonucleotide of 20 nucleosides in length, can be referred to as “fully complementary” for the purposes described herein even though they have different lengths.

As used herein, “complementary oligonucleotides” are those that are capable of base pairing according to the standard Watson-Crick complementarity rules. Specifically, purines will base pair with pyrimidines to form a combination of guanine paired with cytosine (G: C) and adenine paired with either thymine (A: T) in the case of DNA, or adenine paired with uracil (A: U) in the case of RNA. It is understood that two oligonucleotides may hybridize to each other even if they are not completely complementary to each other, provided that each has at least one region that is substantially complementary to the other.

The phrase “contacting a cell with an oligonucleotide,” as used herein, includes contacting a cell with an oligonucleotide, such as a single-stranded oligonucleotide or a double-stranded oligonucleotide (e.g., a single-stranded RNA or a double-stranded RNA that forms a duplex), by methods known in the art. Contacting a cell with an oligonucleotide includes contacting a cell in vitro with the oligonucleotide or contacting a cell in vivo with the oligonucleotide. The contacting may be done directly or indirectly. Thus, for example, the oligonucleotide may be put into physical contact with the cell by the individual performing the method, or alternatively, the RNAi oligonucleotide may be put into a situation that will permit or cause it to subsequently come into contact with the cell. Contacting a cell in vitro may be done, for example, by incubating the cell with the oligonucleotide. Contacting a cell in vivo may be done, for example, by injecting the oligonucleotide into or near the tissue where the cell is located, or by injecting the RNAi oligonucleotide into another area, e.g., the bloodstream or the subcutaneous space, such that the agent will subsequently reach the tissue where the cell to be contacted is located. For example, the oligonucleotide may contain and/or be coupled to a ligand that directs the oligonucleotide to a site of interest or may be integrated into a vector (e.g., a viral vector) that delivers the oligonucleotide to the target site of interest. Combinations of in vitro and in vivo methods of contacting are also possible. For example, a cell may also be contacted in vitro with an oligonucleotide and subsequently transplanted into a subject.

The term “contiguous nucleobase region” refers to a region of an oligonucleotide (e.g., the antisense strand of a RNAi oligonucleotide) that is complementary to a target nucleic acid. The term may be used interchangeably herein with the term “contiguous nucleotide sequence” or “contiguous nucleobase sequence.” In some embodiments, all of the nucleotides of the oligonucleotide are present in the contiguous nucleotide or nucleoside region. In some embodiments, the oligonucleotide includes the contiguous nucleotide region and may optionally include further nucleotide(s) or nucleoside(s). The nucleotide linker region may or may not be complementary to the target nucleic acid. The internucleoside linkages present between the nucleotides of the contiguous nucleotide region may include phosphorothioate internucleoside linkages. Additionally, the contiguous nucleotide region may include one or more sugar-modified nucleosides.

As used herein, the term “deoxyribonucleotide” refers to a nucleotide having a hydrogen in place of a hydroxyl at the 2′ position of its pentose sugar as compared with a ribonucleotide. A modified deoxyribonucleotide is a deoxyribonucleotide having one or more modifications or substitutions of atoms other than at the 2′ position, including modifications or substitutions in or of the sugar, phosphate group or base.

As used herein, the term “disease” refers to an interruption, cessation, or disorder of body functions, systems, or organs. Diseases or disorders of interest include those that would benefit from treatment with an oligonucleotide as described herein (e.g., a single-stranded or a double-stranded RNA construct which forms a duplex as described herein) that is targeted to C3, such as by a treatment method described herein. Non-limiting examples of diseases or disorders mediated by or associated with complement pathway activation or dysregulation that can be treated using the compositions and methods described herein include, for example, cutaneous disorders, neurological disorders, nephrology disorders, acute care, rheumatic disorders, pulmonary disorders, dermatological disorders, hematologic disorders, and ophthalmic disorders, such as e.g., paroxysmal nocturnal hemoglobinuria (PNH), atypical hemolytic uremic syndrome (aHUS), IgA nephropathy, lupus nephritis, C3 glomerulopathy (C3G), dermatomyositis/autoimmune myositis, systemic sclerosis, demyelinating polyneuropathy, pemphigus, membranous nephropathy, focal segmental glomerular sclerosis (FSGS), bullous pemphigoid, epidermolysis bullosa acquisita (EBA), mucus membrane pemphigoid, ANCA vasculitis, hypocomplementemic urticarial vasculitis, immune complex small vessel vasculitis, cutaneous small vessel vasculitis, autoimmune necrotizing myopathy, rejection of a transplanted organ, such as kidney, liver, heart or lung transplant rejection, including antibody mediated rejection (AMR), such as chronic AMR (CAMR), antiphospholipid (aPL) Ab syndrome, glomerulonephritis, asthma, dense deposit disease (DDD), age related macular degeneration (AMD), systemic lupus erythematosus (SLE), rheumatoid arthritis (RA), severe refractory RA, felty syndrome, multiple sclerosis (MS), traumatic brain injury (TBI), spinal cord injury, ischemia reperfusion injury, preeclampsia, delayed graft function in acute kidney injury (DGF-AKI), cardiopulmonary bypass-associated acute kidney injury, hypoxic-ischemic encephalopathy, dialysis-induced thrombosis, Takayasu arteritis, relapsing polychondritis, acute/prophylactic graft vs. host disease, chronic graft vs. host disease, beta thalassemia, stem cell transplant-associated thrombotic microangiopathy, biliary atresia, inflammatory liver disease, Behcet's disease, ischemic stroke, intracerebral hemorrhage, scleroderma, scleroderma renal crisis, scleroderma-associated interstitial lung disease (SSC-ILD), sickle cell disease, autosomal dominant polycystic kidney disease (ADPKD), chemotherapy-induced peripheral neuropathy (CIPN), diabetic neuropathy, amyotrophic lateral sclerosis (ALS), diabetic nephropathy, diabetic retinopathy, geographic atrophy, pulmonary arterial hypertension, refractory severe asthma, chronic obstructive pulmonary disease, idiopathic pulmonary fibrosis (IPF), chronic lung allograft dysfunction, pulmonary morbidities in cystic fibrosis, hidradenitis suppurativa, nonalcoholic fatty liver disease (NASH), ankylosing spondylitis, hematopoietic stem cell transplantation-associated thrombotic microangiopathy (HSCT-TMA) (prevention), coronary artery disease, atherosclerosis, osteoporosis (prevention), osteoarthritis, high risk drusen, inflammatory bowel disease, ulcerative colitis, interstitial cystitis, dialysis induced complement activation, pyoderma gangrenosum, chronic heart failure, autoimmune myocarditis, nasal polyposis, acute and chronic pancreatitis, atherosclerosis, eosinophilic esophagitis, eosinophilic granulomatosis, hypereosinohilic syndrome, wound healing, and thrombotic thrombocytopenia purpura (TTP).

As used herein, the term “duplex,” in reference to nucleic acids (e.g., oligonucleotides), refers to a structure formed through complementary base pairing of two antiparallel sequences of nucleotides.

As used herein, the terms “effective amount,” “therapeutically effective amount,” and “a “sufficient amount” of an agent (e.g., an RNAi oligonucleotide described herein) that reduces the level and/or activity of C3 (e.g., in a cell or a subject) refers to a quantity sufficient to, when administered to the subject, including a human, effect beneficial or desired results, including clinical results, and, as such, an “effective amount” or synonym thereto depends on the context in which it is being applied. For example, in the context of treating a disease associated with complement pathway activation or dysregulation, it is an amount of the agent that reduces the level and/or activity of C3 sufficient to achieve a treatment response as compared to the response obtained without administration of the agent that reduces the level and/or activity of C3. The amount of a given agent that reduces the level and/or activity of C3 described herein that will correspond to such an amount will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent that reduces the level and/or activity of C3 of the present disclosure is an amount which results in a beneficial or desired result in a subject as compared to a control. As defined herein, a therapeutically effective amount of an agent that reduces the level and/or activity of C3 of the present disclosure may be readily determined by one of ordinary skill by routine methods known in the art. Dosage regimen may be adjusted to provide the optimum therapeutic response.

As used herein, the term “excipient” refers to a non-therapeutic agent that may be included in a composition, for example, to provide or contribute to a desired consistency or stabilizing effect.

“G,” “C,” “A,” “T,” and “U” each generally stand for a nucleotide that contains guanine, cytosine, adenine, thymidine, and uracil as a base, respectively, but may include alternative sugar moieties in addition to ribose and deoxyribose. It is also understood that the term “nucleotide” can also refer to an alternative nucleotide, as further detailed below, or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of an oligonucleotide featured in the disclosure by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the disclosure.

As used herein, the term “inhibitor” refers to any agent which reduces the level and/or activity of a protein (e.g., C3). Non-limiting examples of inhibitors include oligonucleotides (e.g., RNAi oligonucleotides, e.g., dsRNA, siRNA, or shRNA). The term “reducing,” as used herein, is used interchangeably with “silencing,” “downregulating,” “suppressing,” and other similar terms, and includes any level of reduction by 5% or more (e.g., 10%, 15%, 25%, 35%, 50%, 75%, and 100%). The typical level of C3 protein found in serum in healthy humans is about 75-175 mg/dL (e.g., 75-100 mg/dL, 75-125 mg/L, 75-150 mg/dL, 150 mg/dL-175 mg/dL, 125-175 mg/dL, and 100-175 mg/dL).

By “level” is meant a level or activity of a protein, or mRNA encoding the protein (e.g., C3), optionally as compared to a reference. The reference can be any useful reference, as defined herein. By a “decreased level” or an “increased level” of a protein is meant a decrease or increase in protein level, respectively, as compared to a reference (e.g., a decrease or an increase of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more, e.g., as compared to a reference; a decrease or an increase of more than about 10%, about 15%, about 20%, about 50%, about 75%, about 100%, or about 200%, e.g., as compared to a reference; a decrease or an increase by less than about 0.01-fold, about 0.02-fold, about 0.1-fold, about 0.3-fold, about 0.5-fold, about 0.8-fold, or less, e.g., as compared to a reference; or a decrease or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more, e.g., as compared to a reference). A level of a protein or mRNA may be expressed in mass/vol (e.g., g/dL, mg/mL, μg/mL, ng/ml) or percentage relative to total protein or mRNA in a sample.

As used herein the term, “loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer or in a cell), the two antiparallel regions, which flank the unpaired region, hybridize to form a duplex (referred to as a “stem”).

As used herein, the term “modified internucleotide linkage” refers to an internucleotide linkage having one or more chemical modifications compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

As used herein, the term “modified nucleotide” refers to a nucleotide having one or more chemical modifications compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

A “nicked tetraloop structure” is a structure of a RNAi oligonucleotide characterized by the presence of separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand. The nicked tetraloop structure causes a single break in the nucleotides of the sense and antisense strands, such that they are no longer joined at that site by a covalent linkage.

The terms “nucleobase” and “base” include the purine (e.g., adenine and guanine) and pyrimidine (e.g., uracil, thymine, and cytosine) moieties present in nucleosides and nucleotides which form hydrogen bonds in nucleic acid hybridization. In the context of the present disclosure, the term nucleobase also encompasses alternative nucleobases which may differ from naturally-occurring nucleobases but are functional during nucleic acid hybridization. In this context “nucleobase” refers to both naturally occurring nucleobases such as adenine, guanine, cytosine, thymidine, uracil, xanthine, and hypoxanthine, as well as alternative nucleobases. Such variants are, for example, described in Hirao et al. (Accounts of Chemical Research, vol. 45: page 2055, 2012) and Bergstrom (Current Protocols in Nucleic Acid Chemistry Suppl. 37 1.4.1, 2009).

The term “nucleoside” refers to a monomeric unit of or an oligonucleotide having a nucleobase and a sugar moiety. A nucleoside may include those that are naturally-occurring as well as alternative nucleosides, such as those described herein. The nucleobase of a nucleoside may be a naturally-occurring nucleobase or an alternative nucleobase. Similarly, the sugar moiety of a nucleoside may be a naturally-occurring sugar or an alternative sugar.

A “nucleotide,” as used herein, refers to a monomeric unit of an oligonucleotide that comprises a nucleoside and an internucleosidic linkage. The internucleosidic linkage may or may not include a phosphate linkage. Similarly, “linked nucleosides” may or may not be linked by phosphate linkages. Many “alternative internucleosidic linkages” are known in the art, including, but not limited to, phosphate, phosphorothioate, and boronophosphate linkages. Alternative nucleosides include bicyclic nucleosides (BNAs) (e.g., locked nucleosides (LNAs) and constrained ethyl (cEt) nucleosides), peptide nucleosides (PNAs), phosphotriesters, phosphorothionates, phosphoramidates, and other variants of the phosphate backbone of native nucleoside, including those described herein.

As used herein, the term “oligonucleotide” refers to a short nucleic acid, e.g., of less than 100 nucleotides in length. An oligonucleotide may be single-stranded or double-stranded. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA, or single-stranded siRNA. In some embodiments, an oligonucleotide is an RNAi oligonucleotide.

As used herein, the term “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of an oligonucleotide (e.g., RNAi oligonucleotide). In certain embodiments, the overhang is a 3′ or 5′ overhang on the antisense strand or sense strand of an oligonucleotide (e.g., RNAi oligonucleotide).

As used herein, the term “patient in need thereof” or “subject in need thereof,” refers to the identification of a subject based on need for treatment of a disease or disorder, such as a disease mediated by complement dysregulation (e.g., dysregulation related to C3, such as dysregulation of one or all of the complement pathways (e.g., alternative, classical, and/or lectin pathways)). A subject can be identified, for example, as having a need for treatment of a disease or disorder (e.g., a disease or disorder associated with complement pathway activation or dysregulation disclosed herein), e.g., based upon an earlier diagnosis by a person of skill in the art (e.g., a physician).

“Percent (%) sequence identity” with respect to a reference oligonucleotide or polypeptide sequence is defined as the percentage of nucleic acids or amino acids in a candidate sequence that are identical to the nucleic acids or amino acids in the reference oligonucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)
where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid. The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “pharmaceutical composition,” as used herein, refers to a composition containing a compound (e.g., an RNAi oligonucleotide) as described herein formulated with a pharmaceutically acceptable excipient, and optionally manufactured or sold with the approval of a governmental regulatory agency as part of a therapeutic regimen for the treatment of disease in a mammal. Pharmaceutical compositions can be formulated, for example, for subcutaneous administration, for intravenous administration (e.g., as a sterile solution free of particulate emboli and in a solvent system suitable for intravenous use); for intrathecal injection; for intracerebroventricular injections; for intraparenchymal injection; for oral administration in unit dosage form (e.g., a tablet, capsule, caplet, gelcap, or syrup); for topical administration (e.g., as a cream, gel, lotion, or ointment; or in any other pharmaceutically acceptable formulation.

As used herein, the term “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include 5′ phosphonates, such as 5′ methylenephosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, for example, US 2019/0177729, the contents of each of which relating to phosphate analogs are incorporated herein by reference. Other modifications have been developed for the 5′ end of oligonucleotides (see, e.g., WO 2011/133871; U.S. Pat. No. 8,927,513; and Prakash et al. (2015), Nucleic Acids Res., 43 (6): 2993-3011, the contents of each of which relating to phosphate analogs are incorporated herein by reference).

The term “probe,” as used herein, refers to any molecule that is capable of selectively binding to a specific sequence, e.g., a nucleic acid molecule, such as an mRNA. Probes can be synthesized using well-known and conventional methods of the art or derived from appropriate biological preparations. Probes may be specifically designed to be labeled. Examples of molecules that can be utilized as probes include, but are not limited to, RNA, DNA, proteins, antibodies, and organic molecules.

As used herein, the term “reduced expression” of a gene refers to a decrease in the amount of RNA transcript or protein encoded by the gene and/or a decrease in the amount of activity of the gene in a cell or subject, as compared to an appropriate reference cell or subject. For example, the act of treating a cell with an RNAi oligonucleotide (e.g., one having an antisense strand that is complementary to C3 mRNA sequence) may result in a decrease in the amount of RNA transcript, protein and/or activity (e.g., encoded by the C3 gene) compared to a cell that is not treated with the RNAi oligonucleotide. Similarly, “reducing expression” as used herein refers to an act that results in reduced expression of a gene (e.g., C3). The reduction in expression can be assessed by a decrease in the serum concentration of C3, as described herein (e.g., relative to, e.g., a cell not contacted with an oligonucleotide described herein). Alternatively, the reduction in expression can be assessed by a decrease in the level of transcription and/or translation of C3 mRNA (e.g., a reduction of at least 1%, 2%, 3%, 4%, 5%, 10%, 15%, 20%, 25%, 30%, 40%, 50%, 55%, or 60% or more, such as a reduction in the range of 1%-60% or more, relative to, e.g., a cell not contacted with an oligonucleotide described herein).

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified oligonucleotide or protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., a disease or disorder associated with complement pathway activation or dysregulation); a subject that has been treated with a compound described herein. In preferred embodiments, the reference sample, standard, or level is matched to the subject sample by at least one of the following criteria: age, weight, sex, disease stage, and overall health. A standard curve of levels of a purified oligonucleotide or protein, e.g., any described herein, within the normal reference range can also be used as a reference.

As used herein, the term “region of complementarity” refers to the region on the antisense strand of an oligonucleotide that is substantially complementary to all or a portion of a gene, primary transcript, a sequence (e.g., a target sequence, e.g., a C3 nucleotide sequence), or processed mRNA, so as to interfere with expression of the endogenous gene (e.g., C3). Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- and/or 3′-terminus of the oligonucleotide (e.g., RNAi oligonucleotide).

As used herein, the term “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

As used herein, the term “RNAi oligonucleotide” refers” to either (a) a double stranded oligonucleotide having a sense strand (passenger) and antisense strand (guide), in which the antisense strand or part of the antisense strand is used by the Argonaute 2 (Ago2) endonuclease in the cleavage of a target mRNA or (b) a single stranded oligonucleotide having a single antisense strand, where that antisense strand (or part of that antisense strand) is used by the Ago2 endonuclease in the cleavage of a target mRNA. In some embodiments, the RNAi oligonucleotide includes a loop region, such as a stem-loop, that contains nucleosides as that term is defined herein. RNAi oligonucleotide includes, for example, dsRNAs, siRNAs, and shRNAs, which mediate the targeted cleavage of an RNA transcript via an RNA-induced silencing complex (RISC) pathway. RNAi oligonucleotide directs the sequence-specific degradation of mRNA through a process known as RNA interference (RNAi). The RNAi oligonucleotide reduces the expression of C3 in a cell, e.g., a cell within a subject, such as a mammalian subject. In general, the majority of the nucleosides of a RNAi oligonucleotide are ribonucleosides, but as described in detail herein, each or both strands can also include one or more non-ribonucleosides, e.g., deoxyribonucleosides and/or alternative nucleosides. A RNAi oligonucleotide is substantially in a duplex form. In some embodiments, the complementary base-pairing of duplex region(s) of a RNAi oligonucleotide is formed between antiparallel sequences of nucleotides of covalently separate nucleic acid strands. In some embodiments, complementary base-pairing of duplex region(s) of an RNAi oligonucleotide is formed between antiparallel sequences of nucleotides of nucleic acid strands that are covalently linked. In some embodiments, complementary base-pairing of duplex region(s) of an RNAi oligonucleotide is formed from a single nucleic acid strand that is folded (e.g., via a hairpin) to provide complementary antiparallel sequences of nucleotides that base pair together. In some embodiments, an RNAi oligonucleotide comprises two covalently separate nucleic acid strands that are fully duplexed with one another. However, in some embodiments, an RNAi oligonucleotide comprises two covalently separate nucleic acid strands that are partially duplexed, e.g., having overhangs at one or both ends. In some embodiments, an RNAi oligonucleotide comprises an antiparallel sequence of nucleotides that are partially complementary, and, thus, may have one or more mismatches, which may include internal mismatches or end mismatches.

The terms “sense strand” and “passenger strand,” as used herein, refer to the strand of an RNAi oligonucleotide that includes a region that is substantially complementary to a region of the antisense strand. The region of the sense strand that is complementary to a region of the antisense strand is at least 85% (e.g., 86%, 87%, 88%, 89%, 90%, 91%, 92%, 93%, 94%, 95%, 96%, 97%, 98%, 99%, and 100%) identical to a portion of the target gene (e.g., the C3 gene). For example, the sense strand may have a region that is at least 85% identical to a portion of SEQ ID NO: 12, such as, e.g., over at least 10 to 36 nucleotides, e.g., over a length of 10 to 31 nucleotides, 10 to 26 nucleotides, 10 to 20 nucleotides, or 10 to 15 nucleotides.

The terms “siRNA” and “short interfering RNA” also known as “small interfering RNA” refer to an RNA agent, optionally a RNAi agent, of about 10-50 nucleotides in length, the strands optionally having overhanging ends comprising, for example 1, 2 or 3 overhanging linked nucleosides, which is capable of directing or mediating RNA interference. Naturally-occurring siRNAs are generated from longer dsRNA molecules (e.g., >25 linked nucleosides in length) by a cell's RNAi machinery (e.g., Dicer or a homolog thereof).

As used herein, the term “strand” refers to a single contiguous sequence of nucleotides linked together through internucleotide linkages (e.g., phosphodiester linkages, phosphorothioate linkages). In some embodiments, a strand has two free ends, e.g., a 5′-end and a 3′-end.

As used herein, the term “subject” refers to any organism to which a composition in accordance with the disclosure may be administered, e.g., for experimental, diagnostic, prophylactic, and/or therapeutic purposes. Typical subjects include any animal (e.g., mammals, such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

A “sugar” or “sugar moiety,” includes naturally occurring sugars having a furanose ring. A sugar also includes an “alternative sugar,” defined as a structure that is capable of replacing the furanose ring of a nucleoside. In certain embodiments, alternative sugars are non-furanose (or 4′-substituted furanose) rings or ring systems or open systems. Such structures include simple changes relative to the natural furanose ring, such as a six-membered ring, or may be more complicated as is the case with the non-ring system used in peptide nucleic acid. Alternative sugars may also include sugar surrogates wherein the furanose ring has been replaced with another ring system such as, for example, a morpholino or hexitol ring system. Sugar moieties useful in the preparation of oligonucleotides having motifs include, without limitation, β-D-ribose, β-D-2′-deoxyribose, substituted sugars (such as 2′,5′ and bis substituted sugars), 4′-S-sugars (such as 4′-S-ribose, 4′-S-2′-deoxyribose and 4′-S-2′-substituted ribose), bicyclic alternative sugars (such as the 2′-O—CH₂-4′ or 2′-O—(CH₂)₂-4′ bridged ribose derived bicyclic sugars) and sugar surrogates (such as when the ribose ring has been replaced with a morpholino or a hexitol ring system).

The type of heterocyclic base and internucleoside linkage used at each position is variable and is not a factor in determining the motif. In most nucleosides having an alternative sugar moiety, the heterocyclic nucleobase is generally maintained to permit hybridization.

As used herein, the term “stem-loop” refers to a region of an oligonucleotide where two regions have a complementary nucleotide sequence when one is read in the 5′ to 3′ direction and the other is read in the 3′ to 5′ direction and nucleotides between the two regions form an unpaired loop. A stem-loop region may also be referred to as a hairpin or a hairpin loop.

As used herein, the term “strand” refers to an oligonucleotide comprising a chain of linked nucleosides” A “strand comprising a nucleobase sequence” refers to an oligonucleotide comprising a chain of linked nucleosides that is described by the sequence referred to using the standard nucleobase nomenclature.

As used herein, the term “synthetic” refers to a nucleic acid or other molecule that is artificially synthesized (e.g., using a machine (e.g., a solid state nucleic acid synthesizer)) or that is otherwise not derived from a natural source (e.g., a cell or organism) that normally produces the molecule.

As used herein, the term “target” or “targeting” refers to an oligonucleotide able to specifically bind to a C3 gene or a C3 mRNA encoding a C3 gene product. For example, it refers to an oligonucleotide able to inhibit said gene or said mRNA (e.g., by reducing the level of protein encoded by the gene or mRNA) by the methods known to those of skill in the art (e.g., in the antisense and RNA interference field).

As used herein, the term “targeting ligand” refers to a molecule (e.g., a carbohydrate, amino sugar, cholesterol, polypeptide or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide or to a vector (e.g., a viral vector) containing an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide or vector facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, the term “tetraloop” refers to a loop that increases stability of an adjacent duplex formed by hybridization of flanking sequences of nucleotides. The increase in stability is detectable as an increase in melting temperature (Tm) of an adjacent stem duplex that is higher than the Tm of the adjacent stem duplex expected, on average, from a set of loops of comparable length consisting of randomly selected sequences of nucleotides. For example, a tetraloop can confer a melting temperature of at least 50° C., at least 55° C., at least 56° C., at least 58° C., at least 60° C., at least 65° C. or at least 75° C. in 10 mM NaHPO₄ to a hairpin comprising a duplex of at least 2 base pairs in length. In some embodiments, a tetraloop may stabilize a base pair in an adjacent stem duplex by stacking interactions. In addition, interactions among the nucleotides in a tetraloop include but are not limited to non-Watson-Crick base pairing, stacking interactions, hydrogen bonding, and contact interactions (Cheong et al., Nature 1990 Aug. 16; 346 (6285): 680-2; Heus and Pardi, Science 1991 Jul. 12; 253 (5016): 191-4). In some embodiments, a tetraloop comprises or consists of 3 to 6 nucleotides and is typically 4 to 5 nucleotides. In certain embodiments, a tetraloop comprises or consists of three, four, five, or six nucleotides, which may or may not be modified (e.g., which may or may not be conjugated to a targeting moiety). In one embodiment, a tetraloop consists of four nucleotides. Any nucleotide may be used in the tetraloop and standard IUPAC-IUB symbols for such nucleotides may be used as described in Cornish-Bowden (1985) Nucl. Acids Res. 13:3021-3030. For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop. (Woese et al., Proc Natl Acad Sci USA. 1990 November; 87 (21): 8467-71; Antao et al., Nucleic Acids Res. 1991 Nov. 11; 19 (21): 5901-5). Examples of DNA tetraloops include the d (GNNA) family of tetraloops (e.g., d (GTTA), the d (GNRA)) family of tetraloops, the d (GNAB) family of tetraloops, the d (CNNG) family of tetraloops, and the d (TNCG) family of tetraloops (e.g., d (TTCG)). See, for example: Nakano et al. Biochemistry, 41 (48), 14281-14292, 2002. SHINJI et al. Nippon Kagakkai Koen Yokoshu VOL. 78th; NO. 2; pg. 731 (2000), which are incorporated by reference herein for their relevant disclosures. In some embodiments, the tetraloop is contained within a nicked tetraloop structure.

A “therapeutically-effective amount” or “prophylactically effective amount” refers to an amount (either administered in a single or in multiple doses) of an oligonucleotide composition of the disclosure (e.g., a RNAi oligonucleotide such as a dsRNA) that produces a desired local or systemic effect. e.g., the treatment of one or more symptoms of a disease resulting from complement pathway activation or dysregulation). Oligonucleotides (e.g., RNAi oligonucleotides) employed in the methods of the present disclosure may be administered in a sufficient amount to produce a reasonable benefit/risk ratio applicable to such treatment.

As used herein, the term “treat” refers to the act of providing care to a subject in need thereof, e.g., through the administration a therapeutic agent (e.g., an oligonucleotide described herein) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom, or contributing factor of a condition (e.g., disease, disorder) experienced by a subject. In some embodiments, the nucleic acid or oligonucleotide (e.g., RNAi oligonucleotide) described herein are used to control the cellular and clinical manifestations of a disorder of the complement pathway, such as, e.g., one or more of the diseases associated with complement pathway activation or dysregulation disclosed herein.

### DETAILED DESCRIPTION

Described herein are oligonucleotides, e.g., RNAi oligonucleotides including sense and antisense strand oligonucleotides, and pharmaceutically acceptable salts thereof, that target complement component (C3), which is known to play a role in complement pathway activation. The oligonucleotides can be administered to decrease the level and/or activity of C3 in a cell (e.g., by hepatocytes). For example, the oligonucleotides can be administered in vivo and can be internalized by a cell (e.g., a hepatocyte; such as by binding to the sialoglycoprotein receptor (ASGPR)). Following cellular internalization, the oligonucleotides can be bound by the RNA-induced silencing complex (RISC) and targeted to C3 mRNA, thereby initiating degradation of the C3 mRNA and blocking translation thereof.

Diseases mediated by complement dysregulation are often a result of complement overactivity. Described herein are methods for treating diseases mediated by, or associated with, complement pathway activation or dysregulation by administration of the oligonucleotides described herein, which reduce the level of expression of C3. Examples of disorders mediated by, or associated with, complement pathway activation or dysregulation that can be treated by the oligonucleotides and compositions described herein include, for example, cutaneous disorders, neurological disorders, nephrology disorders, acute care, rheumatic disorders, pulmonary disorders, dermatological disorders, hematologic disorders, and ophthalmic disorders, such as e.g., paroxysmal nocturnal hemoglobinuria (PNH), atypical hemolytic uremic syndrome (aHUS), IgA nephropathy, lupus nephritis, C3 glomerulopathy (C3G), dermatomyositis/autoimmune myositis, systemic sclerosis, demyelinating polyneuropathy, pemphigus, membranous nephropathy, focal segmental glomerular sclerosis (FSGS), bullous pemphigoid, epidermolysis bullosa acquisita (EBA), mucus membrane pemphigoid, ANCA vasculitis, hypocomplementemic urticarial vasculitis, immune complex small vessel vasculitis, cutaneous small vessel vasculitis, autoimmune necrotizing myopathy, rejection of a transplanted organ, such as kidney, liver, heart or lung transplant rejection, including antibody mediated rejection (AMR), such as chronic AMR (CAMR), antiphospholipid (aPL) Ab syndrome, glomerulonephritis, asthma, dense deposit disease (DDD), age related macular degeneration (AMD), systemic lupus erythematosus (SLE), rheumatoid arthritis (RA), severe refractory RA, felty syndrome, multiple sclerosis (MS), traumatic brain injury (TBI), spinal cord injury, ischemia reperfusion injury, preeclampsia, delayed graft function in acute kidney injury (DGF-AKI), cardiopulmonary bypass-associated acute kidney injury, hypoxic-ischemic encephalopathy, dialysis-induced thrombosis, Takayasu arteritis, relapsing polychondritis, acute/prophylactic graft vs. host disease, chronic graft vs. host disease, beta thalassemia, stem cell transplant-associated thrombotic microangiopathy, biliary atresia, inflammatory liver disease, Behcet's disease, ischemic stroke, intracerebral hemorrhage, scleroderma, scleroderma renal crisis, scleroderma-associated interstitial lung disease (SSC-ILD), sickle cell disease, autosomal dominant polycystic kidney disease (ADPKD), chemotherapy-induced peripheral neuropathy (CIPN), diabetic neuropathy, amyotrophic lateral sclerosis (ALS), diabetic nephropathy, diabetic retinopathy, geographic atrophy, pulmonary arterial hypertension, refractory severe asthma, chronic obstructive pulmonary disease, idiopathic pulmonary fibrosis (IPF), chronic lung allograft dysfunction, pulmonary morbidities in cystic fibrosis, hidradenitis suppurativa, nonalcoholic fatty liver disease (NASH), ankylosing spondylitis, hematopoietic stem cell transplantation-associated thrombotic microangiopathy (HSCT-TMA) (prevention), coronary artery disease, atherosclerosis, osteoporosis (prevention), osteoarthritis, high risk drusen, inflammatory bowel disease, ulcerative colitis, interstitial cystitis, dialysis induced complement activation, pyoderma gangrenosum, chronic heart failure, autoimmune myocarditis, nasal polyposis, acute and chronic pancreatitis, atherosclerosis, eosinophilic esophagitis, eosinophilic granulomatosis, hypereosinohilic syndrome, wound healing, and thrombotic thrombocytopenia purpura (TTP).

The compositions and methods described herein feature an oligonucleotide (e.g., an RNAi oligonucleotide) that include a sense strand and antisense strand, which has substantial sequence identity to a region of the C3 gene.

The oligonucleotide (e.g., RNAi oligonucleotide) can be used to regulate complement pathway activity, e.g., by reducing the level and/or activity of C3 in a cell (e.g., a hepatocyte), such as a cell in a subject (e.g., a human) in need thereof. The overall design targets C3 of the complement pathway and leaves activation (protection) of the other pathways of the alternative, classical, and lectin pathways intact. Accordingly, the disclosure features compositions and methods for treating diseases or disorders mediated by complement pathway activation or dysregulation e.g., diseases or disorders mediated by activation or dysregulation of C3).

### Complement Component 3 Target Sequence

Oligonucleotide-based inhibitors of C3 expression are provided herein that can be used to achieve a therapeutic benefit. Through examination of the C3 mRNA, (see, e.g., Example 3) and in vitro and in vivo testing, it has been discovered that sequences of C3 mRNA are useful as targeting sequences because they are amenable to oligonucleotide-based inhibition. For example, a C3 target sequence can include, or may consist of, a sequence as forth in either of SEQ ID Nos: 13 or 14, which corresponds to nucleotides 4121-4141 and 780-798, respectively, of the Homo sapiens complement C3 with Reference Sequence NM_0.000064.4 (SEQ ID NO: 12). These C3 sequences may be the target sequences of Compound A and Compound B, respectively, and variants thereof described herein that have up to 85% sequence identity thereto. Compounds A and B (and their variants described herein) may also effectively target the Rhesus macaque and Cynomolgus macaques complement C3 with Reference Sequences XM_015122636.2 and XM_005587719.2, respectively. Furthermore, a C3 target sequence can include, or may consist of, a sequence as forth in either of SEQ ID NO: 31 which corresponds to nucleotides 2903-2922 of the Mus musculus complement C3 with Reference Sequence NM_009778.3 (SEQ ID NO: 32), which may be the target of Compound J (e.g., an RNAi oligonucleotide having the sense sequence of SEQ ID NO: 15 and the antisense sequence of SEQ ID NO: 16). Compound J may also target the Rattus norvegicus complement C3 with Reference Sequence NM_016994.2. These regions of C3 mRNA may be targeted using the RNAi oligonucleotides such as the dsRNA agents described herein for purposes of inhibiting C3 mRNA expression and subsequent C3 protein expression.

In some embodiments, the antisense strands of the oligonucleotide (e.g., RNAi oligonucleotide) agents provided herein can be designed to have regions of complementarity to C3 mRNA (e.g., within a target sequence of C3 mRNA) for purposes of targeting the mRNA in cells and inhibiting its expression. The region of complementarity is generally of a suitable length and base content to promote annealing of the oligonucleotide (e.g., RNAi oligonucleotide), or a strand thereof, to C3 mRNA for purposes of inhibiting its transcription. The region of complementarity can be at least 11, e.g., at least 12, at least 13, at least 14, at least 15, at least 16, at least 17, at least 18, at least 19 or at least 20 nucleotides in length. For example, an oligonucleotide provided herein may have a region of complementarity to C3 mRNA that is in the range of 12 to 30 (e.g., 12 to 30, 12 to 22, 15 to 25, 17 to 21, 18 to 27, 19 to 27, or 15 to 30) nucleotides in length. Accordingly, the oligonucleotide provided herein may have a region of complementarity to C3 that is 12, 13, 14, 15, 16, 17, 18, 19, 20, 21, 22, 23, 24, 25, 26, 27, 28, 29 or 30 nucleotides in length. In some instances, the oligonucleotide provided herein may have a region of complementarity to the C3 mRNA that is 19 nucleotides in length. In certain embodiments, the region of complementarity of an oligonucleotide (e.g., an antisense strand of an RNAi oligonucleotide) may be complementary to a contiguous sequence of nucleotide of a sequence as set forth in SEQ ID NO: 12 that is 20 nucleotides in length.

In certain instances, an RNAi oligonucleotide of the present disclosure may include a region of complementarity (e.g., on an antisense strand of an RNAi oligonucleotide) that is at least partially complementary to a sequence as set forth in SEQ ID NO: 12. For example, an oligonucleotide disclosed herein may comprise a region of complementarity (e.g., on an antisense strand of an RNAi oligonucleotide) that is fully complementary to a sequence as set forth in SEQ ID NO: 12. The region of complementarity of an oligonucleotide (e.g., on an antisense strand of an RNAi oligonucleotide) may be complementary to a contiguous sequence of nucleotides of a sequence as set forth in SEQ ID NO: 12 that is in the range of 12 to 20 nucleotides (e.g., 12 to 20, 12 to 18, 12 to 16, 12 to 14, 14 to 20, 14 to 18, 14 to 16, 16 to 20, 16 to 18, or 18 to 20) in length. In some embodiments, the region of complementarity of an oligonucleotide (e.g., on an antisense strand of an RNAi oligonucleotide) may be complementary to a contiguous sequence of nucleotides of a sequence as set forth in SEQ ID NO: 12 that is 19 nucleotides in length. In certain embodiments, the region of complementarity of an oligonucleotide (e.g., an antisense strand of an RNAi oligonucleotide) may be complementary to a contiguous sequence of nucleotide of a sequence as set forth in SEQ ID NO: 12 that is 20 nucleotides in length.

The region of complementarity of an oligonucleotide that is complementary to contiguous nucleotides of a sequence as set forth in SEQ ID NO: 12 may span a portion of the entire length of an antisense strand. For example, the region of complementarity of an oligonucleotide that is complementary to contiguous nucleotides of a sequence as set forth in SEQ ID NO: 12 may span at least 85% (e.g., at least 86%, at least 90%, at least 95%, and at least 99%) of the entire length of the antisense strand. In certain embodiments, the region of complementarity of the oligonucleotide that is complementary to contiguous nucleotides as set forth in SEQ ID NO: 12 may span the entire length of the antisense strand.

The region of complementarity to C3 mRNA may have one or more mismatches as compared with a corresponding sequence of C3 mRNA. For example, a region of complementarity on an oligonucleotide (e.g., an oligonucleotide of 20 to 50 nucleotides in length, such as an oligonucleotide of 20-25 nucleotides in length (e.g., 22 nucleotides in length) may have up to 1, up to 2, up to 3, up to 4, or up to 5 mismatches provided that it maintains the ability to form complementary base pairs with C3 mRNA under appropriate hybridization conditions. Alternatively, a region of complementarity on an oligonucleotide may have no more than 1, no more than 2, no more than 3, no more than 4, or no more than 5 mismatches provided that it maintains the ability to form complementary base pairs with C3 mRNA under appropriate hybridization conditions. If there is more than one mismatch in a region of complementarity, the mismatches may be positioned consecutively (e.g., 2, 3, 4, or 5 in a row) or interspersed throughout the region of complementarity, provided that the oligonucleotide maintains the ability to form complementary base pairs with C3 mRNA under appropriate hybridization conditions. For example, the RNAi oligonucleotide may include a sense oligonucleotide with the sequence of SEQ ID NO: 4 and variants thereof with up to 1, 2, 3, 4, or 5 mismatches relative to the corresponding C3 sequence of SEQ ID NO: 12, or a corresponding antisense sequence of SEQ ID NO: 6 and variants thereof with up to 1, 2, 3, 4, or 5 mismatches relative to the sequence of SEQ ID NO: 4.

### Types of Oligonucleotides

There are a variety of structures of oligonucleotides that are useful for targeting C3 in the methods of the present disclosure, including RNAi, antisense miRNA, shRNA, and others. Any of the structures described herein or elsewhere may be used as a framework to incorporate or target a sequence described herein (e.g., a hotspot sequence of C3, such as those of SEQ ID NOs: 13 or 14).

The compositions described herein, which are oligonucleotides (e.g., RNAi oligonucleotides), encode inhibitory constructs (e.g., nucleic acid vectors encoding the same) that target a C3 mRNA (e.g., SEQ ID NO: 12). The oligonucleotides for reducing the expression of C3 expression may engage RNA interference (RNAi) pathways upstream or downstream of dicer involvement. For example, oligonucleotides (e.g., RNAi oligonucleotides) have been developed with 19-25 nucleotides in lengths and with at least one of the sense or antisense strands having a 3′ overhang between 1 and 5 nucleotides (see, e.g., U.S. Pat. No. 8,372,968, which is incorporated herein by reference). Longer oligonucleotides have also been developed that are processed by dicer to generate active RNAi products (see, e.g., U.S. Pat. No. 8,883,996, which is incorporated herein by reference). Furthermore, extended oligonucleotides (e.g., RNAi oligonucleotides) have been produced where either one or both of the 5′ or the 3′ ends of either one or both of the antisense and sense strands are extended beyond a duplex targeting region, such that either the sense strand or the antisense strand includes a thermodynamically-stabilizing tetraloop structure (see, e.g., U.S. Pat. Nos. 8,513,207 and 8,927,705, as well as WO2010033225, which are incorporated by reference herein for their disclosure of these oligonucleotides). Such structures may include single-stranded extensions on one or both of the 5′ and 3′ ends of the molecule, as well as RNAi extensions.

Additionally, or alternatively, the oligonucleotides provided herein may be designed to engage in the RNA interference pathway downstream of the involvement of dicer, meaning after cleavage by dicer. Such oligonucleotides may have an overhang which includes 1, 2, or 3 nucleotides at the 3′ end of the sense strand. Such oligonucleotides, such as siRNAs, may include a 22-nucleotide guide strand that is antisense to a target RNA (e.g., SEQ ID NO: 13 and 14) and a complementary passenger strand, in which both strands anneal to form a 20-bp duplex and 2 nucleotide overhangs at either or both 3′ ends. Longer oligonucleotide designs are also available, including oligonucleotides having a guide strand of 23 nucleotides and a passenger strand of 21 nucleotides, where there is a blunt end on the 3′-end of passenger strand and 5′-end of the guide strand and a two nucleotide 3′-guide strand overhang on the left side of the molecule 5′-end of the passenger strand and 3′-end of the guide strand. In such molecules, there is a 21 base pair duplex region (see U.S. Pat. Nos. 9,012,138, 9,012,621, and 9,193,753, which are incorporated by reference herein for their disclosure regarding longer oligonucleotides).

The oligonucleotides as disclosed herein may include sense and antisense strands that are both in the range of 17 to 26 (e.g., 17 to 26, 20 to 25, or 21-23) nucleotides in length. For example, an oligonucleotide disclosed herein may include a sense and antisense strand that are both in the range of 19-22 nucleotide in length. The sense and antisense strands may also be of equal length. Alternatively, an oligonucleotide may include sense and antisense strands, such that there is a 3′-overhang on either the sense strand or the antisense strand, or both the sense and antisense strand. For example, the 3′ overhang on the sense, antisense, or both sense and antisense strands may be 1 or 2 nucleotides in length. In some embodiments, the oligonucleotide has an antisense strand of 22 nucleotides and a sense strand of 20 nucleotides, where there is a blunt end on the “right” side of the molecule (i.e., at the 3′-end of the passenger strand and the 5′-end of the guide strand) and a two nucleotide 3′-guide strand overhang on the “left” side of the molecule (i.e., at the 5′-end of the passenger strand and the 3′-end of the guide strand). In such molecules, there may be, e.g., a 20 base pair duplex region.

Other oligonucleotide designs for use with the compositions and methods disclosed herein include, e.g., 16-mer siRNAs (see, e.g., Nucleic Acids in Chemistry and Biology. Blackburn (ed.), Royal Society of Chemistry, 2006), shRNAs (e.g., having 19 bp or shorter stems; see, e.g., Moore et al. Methods Mol. Biol. 2010; 629:141-158), blunt siRNAs (e.g., of 19 bps in length; see: e.g., Kraynack and Baker, RNA Vol. 12, p 163-176 (2006)), asymmetrical siRNAs (aiRNA; see, e.g., Sun et al., Nat. Biotechnol. 26, 1379-1382 (2008)), asymmetric shorter-duplex siRNA (see, e.g., Chang et al., Mol Ther. 2009 April; 17 (4): 725-32), fork siRNAs (see, e.g., Hohjoh, FEBS Letters, Vol 557, issues 1-3; January 2004, p 193-198), single-stranded siRNAs (Elsner; Nature Biotechnology 30, 1063 (2012)), dumbbell-shaped circular siRNAs (see, e.g., Abe et al. J Am Chem Soc 129:15108-15109 (2007)), and small internally segmented interfering RNA (siRNA; see, e.g., Bramsen et al., Nucleic Acids Res. 2007 September; 35 (17): 5886-5897). Each of the foregoing references is incorporated by reference in its entirety for the related disclosures therein. Further non-limiting examples of an oligonucleotide structures that may be used in some embodiments to reduce or inhibit the expression of C3 are microRNA (miRNA), short hairpin RNA (shRNA), and short siRNA (see, Hamilton et al., Embo J., 2002, 21 (17): 4671-4679; see also U.S. Pat. Appln Pub. No. 2009/0099115).

### Oligonucleotides

Oligonucleotides (e.g., RNAi oligonucleotides) for targeting C3 expression via the RNAi pathway generally have a sense strand and an antisense strand that form a duplex with one another. The oligonucleotides (e.g., RNAi oligonucleotides) may be single-stranded or double-stranded ribonucleic acids (dsRNA). Furthermore, the sense and antisense strands may not be covalently linked; for example, the oligonucleotide may be nicked between the sense and antisense strand. The oligonucleotides (e.g., RNAi oligonucleotides) may be in the form of a pharmaceutically acceptable salt. For example, the oligonucleotide (e.g., RNAi oligonucleotide) may be in the form of a sodium salt.

The foregoing oligonucleotide (e.g., RNAi oligonucleotide) sequences are represented as RNA sequences that can be synthesized within the cell; however, these sequences may also be represented as corresponding DNA (e.g., cDNA) that can be incorporated into a vector of the disclosure. One skilled in the art would understand that the cDNA sequence is equivalent to the mRNA sequence, except for the substitution of uridines with thymidines, and can be used for the same purpose herein, i.e., the generation of an antisense oligonucleotide for inhibiting the expression of C3 mRNA. In the case of DNA, the polynucleotide containing the antisense nucleic acid is a DNA sequence. The DNA sequence may correspond to the antisense strand of Compound A or Compound B and may have the polynucleotide sequence of SEQ ID NO: 34 or SEQ ID NO: 35, respectively, or may have at least 85% or more sequence identity thereto. The DNA sequence may correspond to the sense strand of Compound A or Compound B and may have the polynucleotide sequence of SEQ ID NO: 33 or SEQ ID NO: 35, respectively, or may have at least 85% or more sequence identity thereto. In the case of RNA vectors, the transgene cassette incorporates the RNA equivalent of the antisense DNA sequences described herein.

In certain embodiments, the sense strand may include an oligonucleotide sequence having at least 85% (e.g., at least 87%, at least 90%, at least 95%, at least 97%, and at least 99%) sequence identity to SEQ ID NO: 4 or SEQ ID NO: 5. For example, the sense strand may include an oligonucleotide sequence of SEQ ID NO: 4, as in the case of Compound B. In other embodiments, the sense strand may include an oligonucleotide sequence having at least 85% (e.g., at least 87%, at least 90%, at least 95%, at least 97%, and at least 99%) sequence identity to SEQ ID NO: 1 or SEQ ID NO: 2. For example, the sense strand may include an oligonucleotide sequence of SEQ ID NO: 1, as in the case of Compound A.

In some embodiments, the antisense strand may include an oligonucleotides sequence having at least 85% (e.g., at least 87%, at least 90%, at least 95%, at least 97%, and at least 99%) sequence identity to SEQ ID NO: 6. In other embodiments, the antisense strand may include an oligonucleotide sequence having at least 85% (e.g., at least 87%, at least 90%, at least 95%, at least 97%, and at least 99%) sequence identity to SEQ ID NO: 3. For example, the antisense strand may include an oligonucleotide sequence of SEQ ID NO: 6, as in the case of Compound B, and/or the antisense strand may include an oligonucleotide sequence of SEQ ID NO: 3, as in the case of Compound A.

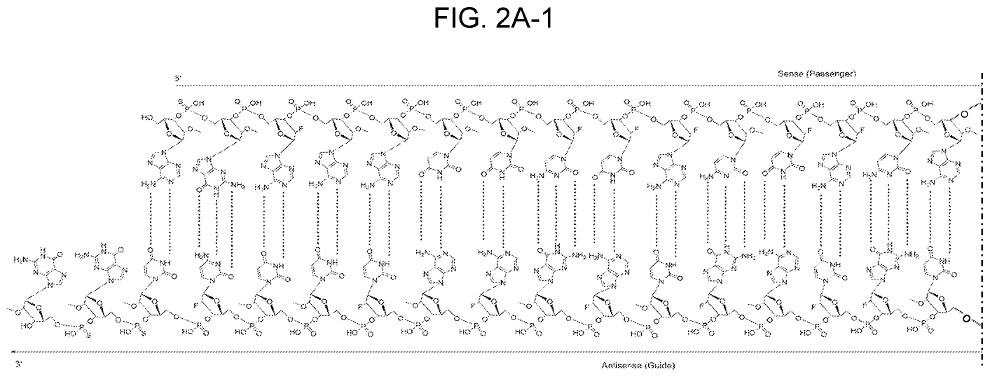

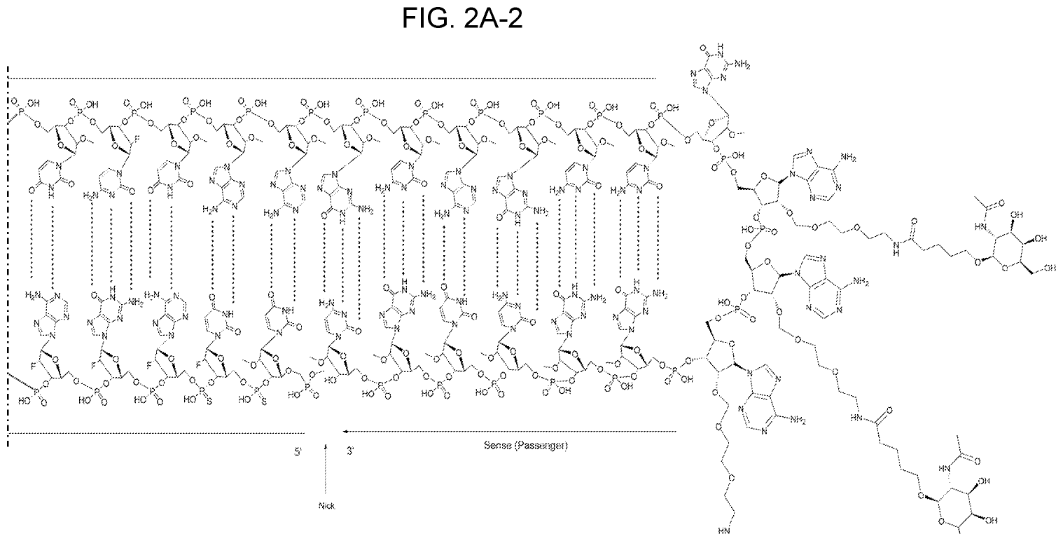

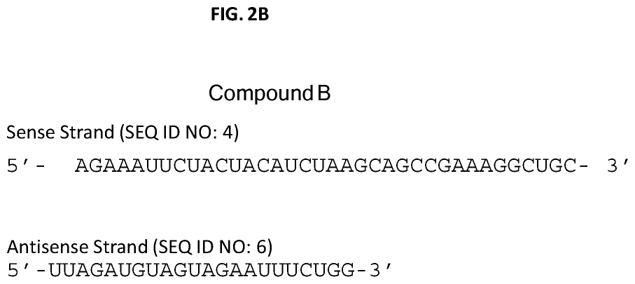

Furthermore, the sense strand may include an oligonucleotide sequence having at least 85% (e.g., at least 87%, at least 90%, at least 95%, at least 97%, and at least 99%) sequence identity to SEQ ID NO: 4 or SEQ ID NO: 5 and the antisense strand may include an oligonucleotide sequence having at least 85% (e.g., at least 87%, at least 90%, at least 95%, at least 97%, and at least 99%) sequence identity to SEQ ID NO: 6. The oligonucleotide (e.g., RNAi oligonucleotide) may contain a sense strand that includes an oligonucleotide sequence of SEQ ID NO: 4 or SEQ ID NO: 5 and an antisense strand that includes an oligonucleotide sequence of SEQ ID NO: 6, as shown for Compound B in FIG. 2B.

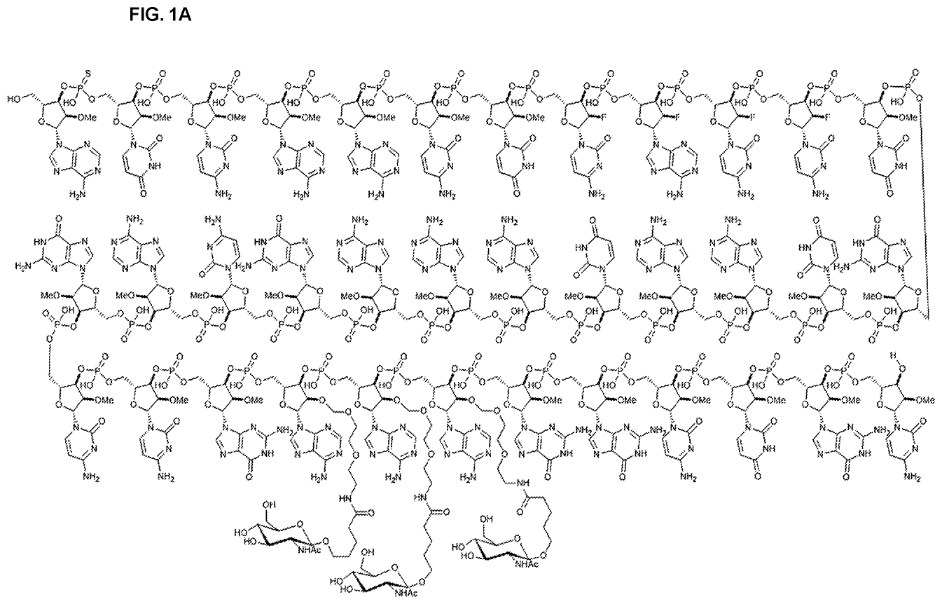

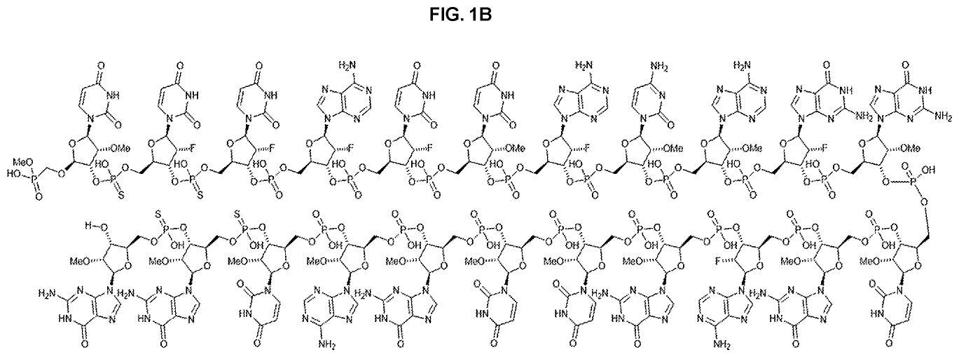

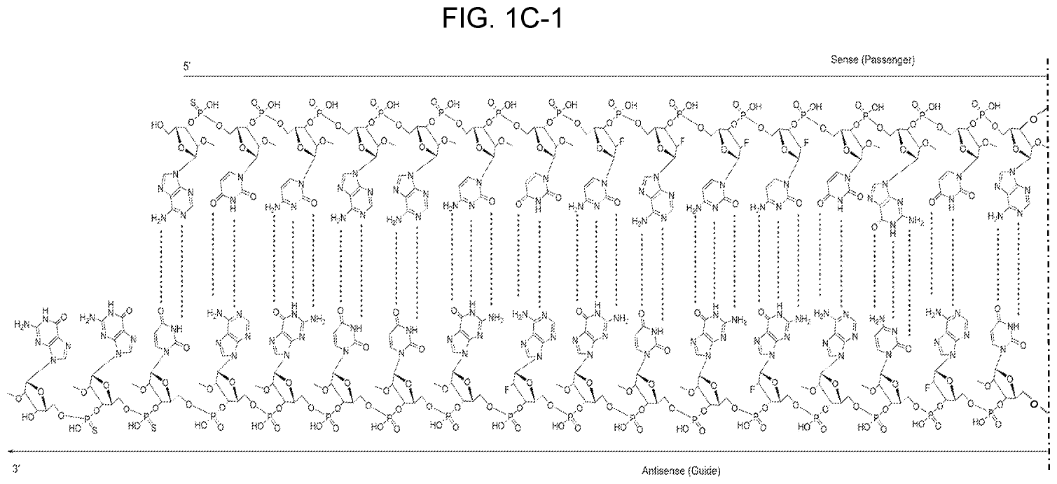

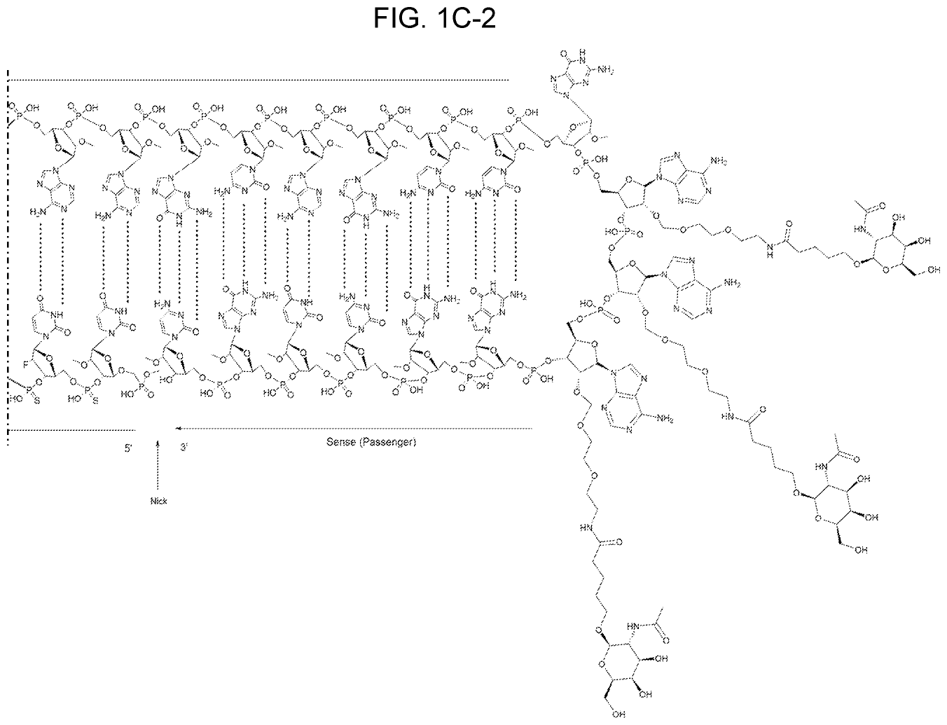

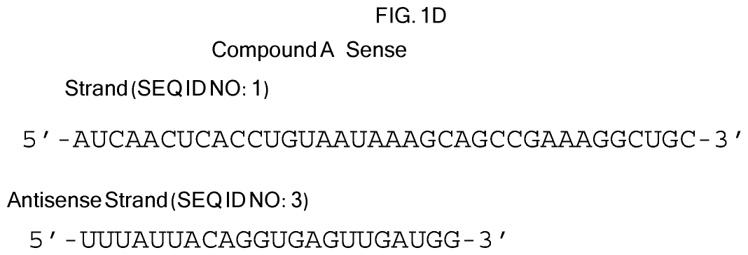

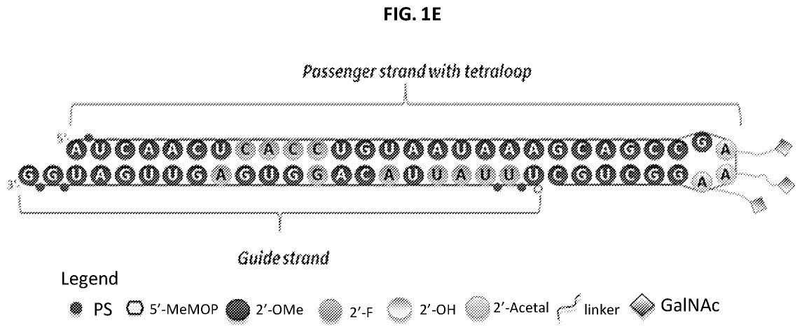

Additionally, the sense strand may include an oligonucleotide sequence having at least 85% (e.g., at least 87%, at least 90%, at least 95%, at least 97%, and at least 99%) sequence identity to SEQ ID NO: 1 or SEQ ID NO: 2 and the antisense strand may include an oligonucleotide sequence having at least 85% (e.g., at least 87%, at least 90%, at least 95%, at least 97%, and at least 99%) sequence identity to SEQ ID NO: 3. Furthermore, the oligonucleotide (e.g., RNAi oligonucleotide) may contain a sense strand that includes an oligonucleotide sequence of SEQ ID NO: 1 or SEQ ID NO: 2 and an antisense strand that includes an oligonucleotide sequence of SEQ ID NO: 3, as shown for Compound A in FIGS. 1D and 1E. An oligonucleotide provided herein may include a sense strand having a sequence as set forth in any one of SEQ ID NOs: 1, 2, 4, and 5 and an antisense strand including a complementary sequence selected from SEQ ID NOs: 3 and 6.

Furthermore, the sense strand may include an oligonucleotide sequence of SEQ ID NO: 37 and the antisense strand may include an oligonucleotide sequence of SEQ ID NO: 38 as shown below.

Sense Strand (SEQ ID NO: 37):
5′ mA-S-mU-mC-mA-mA-mC-mU-fC-fA-fC-fC-mU-mG-mU-
mA-mA-mU-mA-mA-mA-mG-mC-mA-mG-mC-mC-mG-[ademA-
GalNAc]-[ademA-GalNAc]-[ademA-GalNAc]-mG-mG-mC-
mU-mG-mC 3′

hybridized to:

Antisense Strand (SEQ ID NO: 38):
5′ [MePhosphonate-40-mU]-S-fU-S-fU-fA-fU-mU-fA-mC-
mA-fG-mG-mU-mG-fA-mG-mU-mU-mG-mA-mU-S-mG-S-mG 3′

in which mX is a 2′-O-methyl ribonucleotide, fX is a 2′-fluoro-deoxyribonucleotide, [ademA-GalNAc] is a 2′-O-GalNAc-modified adenosine, [MePhosphonate-40-mU] is a 4′-O-monomethylphosphonate-2′-O-methyl uridine, “-” denotes a phosphodiester linkage, and “—S—” denotes a phosphorothioate linkage as shown in FIG. 1E. In some embodiments, the antisense stand may be a pharmaceutically acceptable salt (e.g., a sodium salt) of SEQ ID NO: 38. In some embodiments, the sense stand may be a pharmaceutically acceptable salt (e.g., a sodium salt) of SEQ ID NO: 37.

Furthermore, the sense strand may include an oligonucleotide sequence having at least 85% (e.g., at least 87%, at least 90%, at least 95%, at least 97%, and at least 99%) sequence identity to SEQ ID NO: 1 and the antisense strand may include an oligonucleotide sequence having at least 85% (e.g., at least 87%, at least 90%, at least 95%, at least 97%, and at least 99%) sequence identity to SEQ ID NO: 3. For example, the oligonucleotide (e.g., RNAi oligonucleotide) may contain a sense strand that includes an oligonucleotide sequence of SEQ ID NO: 1 and an antisense strand that includes an oligonucleotide sequence of SEQ ID NO: 3, as shown for Compound A. The sense strand may include an oligonucleotide sequence having at least 85% (e.g., at least 87%, at least 90%, at least 95%, at least 97%, and at least 99%) sequence identity to SEQ ID NO: 4 and the antisense strand may include an oligonucleotide sequence having at least 85% (e.g., at least 87%, at least 90%, at least 95%, at least 97%, and at least 99%) sequence identity to SEQ ID NO: 6. For example, the oligonucleotide (e.g., RNAi oligonucleotide) may contain a sense strand that includes an oligonucleotide sequence of SEQ ID NO: 4 and an antisense strand that includes an oligonucleotide sequence of SEQ ID NO: 6, as shown for Compound B. The sense strand may include an oligonucleotide sequence having at least 85% (e.g., at least 87%, at least 90%, at least 95%, at least 97%, and at least 99%) sequence identity to SEQ ID NO: 17 and the antisense strand may include an oligonucleotide sequence having at least 85% (e.g., at least 87%, at least 90%, at least 95%, at least 97%, and at least 99%) sequence identity to SEQ ID NO: 18. For example, the oligonucleotide (e.g., RNAi oligonucleotide) may contain a sense strand that includes an oligonucleotide sequence of SEQ ID NO: 17 and an antisense strand that includes an oligonucleotide sequence of SEQ ID NO: 18, as shown for Compound C. The sense strand may include an oligonucleotide sequence having at least 85% (e.g., at least 87%, at least 90%, at least 95%, at least 97%, and at least 99%) sequence identity to SEQ ID NO: 19 and the antisense strand may include an oligonucleotide sequence having at least 85% (e.g., at least 87%, at least 90%, at least 95%, at least 97%, and at least 99%) sequence identity to SEQ ID NO: 20. For example, the oligonucleotide (e.g., RNAi oligonucleotide) may contain a sense strand that includes an oligonucleotide sequence of SEQ ID NO: 19 and an antisense strand that includes an oligonucleotide sequence of SEQ ID NO: 20, as shown for Compound D. The sense strand may include an oligonucleotide sequence having at least 85% (e.g., at least 87%, at least 90%, at least 95%, at least 97%, and at least 99%) sequence identity to SEQ ID NO: 21 and the antisense strand may include an oligonucleotide sequence having at least 85% (e.g., at least 87%, at least 90%, at least 95%, at least 97%, and at least 99%) sequence identity to SEQ ID NO: 22. For example, the oligonucleotide (e.g., RNAi oligonucleotide) may contain a sense strand that includes an oligonucleotide sequence of SEQ ID NO: 21 and an antisense strand that includes an oligonucleotide sequence of SEQ ID NO: 22, as shown for Compound E. The sense strand may include an oligonucleotide sequence having at least 85% (e.g., at least 87%, at least 90%, at least 95%, at least 97%, and at least 99%) sequence identity to SEQ ID NO: 23 and the antisense strand may include an oligonucleotide sequence having at least 85% (e.g., at least 87%, at least 90%, at least 95%, at least 97%, and at least 99%) sequence identity to SEQ ID NO: 24. For example, the oligonucleotide (e.g., RNAi oligonucleotide) may contain a sense strand that includes an oligonucleotide sequence of SEQ ID NO: 23 and an antisense strand that includes an oligonucleotide sequence of SEQ ID NO: 24, as shown for Compound F. The sense strand may include an oligonucleotide sequence having at least 85% (e.g., at least 87%, at least 90%, at least 95%, at least 97%, and at least 99%) sequence identity to SEQ ID NO: 25 and the antisense strand may include an oligonucleotide sequence having at least 85% (e.g., at least 87%, at least 90%, at least 95%, at least 97%, and at least 99%) sequence identity to SEQ ID NO: 26. For example, the oligonucleotide (e.g., RNAi oligonucleotide) may contain a sense strand that includes an oligonucleotide sequence of SEQ ID NO: 25 and an antisense strand that includes an oligonucleotide sequence of SEQ ID NO: 26, as shown for Compound G. The sense strand may include an oligonucleotide sequence having at least 85% (e.g., at least 87%, at least 90%, at least 95%, at least 97%, and at least 99%) sequence identity to SEQ ID NO: 27 and the antisense strand may include an oligonucleotide sequence having at least 85% (e.g., at least 87%, at least 90%, at least 95%, at least 97%, and at least 99%) sequence identity to SEQ ID NO: 28. For example, the oligonucleotide (e.g., RNAi oligonucleotide) may contain a sense strand that includes an oligonucleotide sequence of SEQ ID NO: 27 and an antisense strand that includes an oligonucleotide sequence of SEQ ID NO: 28, as shown for Compound H. The sense strand may include an oligonucleotide sequence having at least 85% (e.g., at least 87%, at least 90%, at least 95%, at least 97%, and at least 99%) sequence identity to SEQ ID NO: 29 and the antisense strand may include an oligonucleotide sequence having at least 85% (e.g., at least 87%, at least 90%, at least 95%, at least 97%, and at least 99%) sequence identity to SEQ ID NO:

30. For example, the oligonucleotide (e.g., RNAi oligonucleotide) may contain a sense strand that includes an oligonucleotide sequence of SEQ ID NO: 29 and an antisense strand that includes an oligonucleotide sequence of SEQ ID NO: 30, as shown for Compound I. The sense strand may include an oligonucleotide sequence having at least 85% (e.g., at least 87%, at least 90%, at least 95%, at least 97%, and at least 99%) sequence identity to SEQ ID NO: 15 and the antisense strand may include an oligonucleotide sequence having at least 85% (e.g., at least 87%, at least 90%, at least 95%, at least 97%, and at least 99%) sequence identity to SEQ ID NO: 16. For example, the oligonucleotide (e.g., RNAi oligonucleotide) may contain a sense strand that includes an oligonucleotide sequence of SEQ ID NO: 15 and an antisense strand that includes an oligonucleotide sequence of SEQ ID NO: 16, as shown for Compound J. See Table 1 for examples of sense strand and antisense strand pairs.

TABLE 1
RNAi oligonucleotides targeting C3 mRNA

| | | | | | Target |
|---|---|---|---|---|---|
| | | SEQ | | SEQ | sequence |
| Con- | | ID | Antisense | ID | nucleotide |
| struct | Sense Strand | NO: | Strand | NO: | (C3 mRNA) |
| 1 | AUCAACUCACCU | 1 | UUUAUUACA | 3 | 4122-4141 |
| | GUAAUAAAGCAG | | GGUGAGUU | | of SEQ ID |
| | CCGAAAGGCUGC | | | | NO: 12 |
| 2 | AUCAACUCACCU | 2 | GAUGG | | |
| | GUAAUAAA | | | | |
| 3 | AGAAAUUCUACU | 4 | UUAGAUGUA | 6 | 780-798 of |
| | ACAUCUAAGCAG | | GUAGAAUUU | | SEQ ID NO: |
| | CCGAAAGGCUGC | | CUGG | | 12 |
| 4 | AGAAAUUCUACU | 5 | UUAGAUGUA | 6 | |
| | ACAUCUAA | | GUAGAAUUU | | |
| | | | CUGG | | |
| 5 | AGGAAUGAGAAU | 15 | UUUUGUUGA | 16 | 2903-2922 |
| | CAACAAAAAGCA | | UUCUCAUUC | | of SEQ ID |
| | GCCGAAAGGCUG | | CUG | | NO: 32 |
| | C | | | | |

The oligonucleotide (e.g., RNAi oligonucleotide) includes a duplex region between the sense strand and the antisense strand. The duplex formed between the sense and antisense strand may be between 10 and 30 nucleotides in length (e.g., 10, 11, 12, 13, 14, 15, 16, 17, 18, 19, 20, 21, 22, 23, 24, 25, 26, 27, 28, 29, and 30 nucleotides in length). Accordingly, the duplex formed between a sense and antisense strand may be may between 15 and 25 nucleotides in length (e.g., 15, 16, 17, 18, 19, 20, 21, 22, 23, 24, and 25 nucleotides in length). In some embodiments, the duplex region may be 20 nucleotides in length.

The region on the sense strand that forms a duplex with the antisense strand may have a nucleotide sequence that is at least 85% identical (e.g., 86%, 87%, 88%, 89%, 90%, 91%, 92%, 93%, 94%, 95%, 96%, 97%, 98%, 99%, or more) to the oligonucleotide sequences of either of SEQ ID NOs: 2 and 5. For example, the region on the sense strand that forms a duplex with the antisense strand may have an oligonucleotide sequence of either of SEQ ID NOs: 2 and 5.

Furthermore, a duplex formed between a sense and antisense strand may not span the entire length of the sense strand and/or antisense strand.

The oligonucleotide (e.g., RNAi oligonucleotide) may include a sense strand that is longer than 22 nucleotides (e.g., 23, 24, 25, 26, 27 28, 29, 30, 31, 32, 33, 34, 35, 36, 37, 38, 39 or 40 nucleotides in length), such as a 36-nucleotide sense strand, and an antisense strand that is 18-36 nucleotides in length, such as a 22-nucleotide antisense strand. The oligonucleotide (e.g., RNAi oligonucleotide) has a length such that, when acted upon by a dicer enzyme, the result is an antisense strand that is incorporated into the mature RISC.

The oligonucleotides provided herein may have one 5′ end that is thermodynamically less stable compared to the other 5′ end. The oligonucleotides provided herein may be an asymmetric oligonucleotide that includes a blunt end at the 3′ end of a sense strand and an overhang at the 3′ end of an antisense strand. The 3′ overhang on an antisense strand may be 1-8 nucleotides in length (e.g., 1, 2, 3, 4, 5, 6, 7 or 8 nucleotides in length). For example, the 3′ overhang on the antisense strand may be two nucleotides in length. Typically, an oligonucleotide for RNAi has a two-nucleotide overhang on the 3′ end of the antisense, guide, strand; however, other overhangs are possible. In other embodiments, the 3′ overhang may have a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5, or 6 nucleotides. In some instances, the oligonucleotides may have an overhang on the 5′ end. The overhang may be a 5′ overhang including a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides.

The two terminal nucleotides on the 3′ end of an antisense strand may be modified. In certain embodiments. The two terminal nucleotides on the 3′ end of the antisense strand may be complementary with the target C3 mRNA. Alternatively, the two terminal nucleotides on the 3′ end of the antisense strand may not be complementary with the target C3 mRNA. In some embodiments, the two terminal nucleotides on the 3′ end of the antisense strand may be GG. Typically, one or both of the two terminal GG nucleotides on each 3′ end of an oligonucleotide is not complementary with the target.

There may be one or more (e.g., 1, 2, 3, 4, 5) mismatches in complementarity between a sense and antisense strand. If there is more than one mismatch between a sense and antisense strand, they may be positioned consecutively (e.g., 2, 3 or more in a row), or interspersed throughout the region of complementarity. For instance, the 3′ end of the sense strand may contain one or more mismatches. Accordingly, two mismatches may be incorporated at the 3′ end of the sense strand. Base mismatches or destabilization of segments at the 3′-end of the sense strand of the oligonucleotide may improve the potency of synthetic duplexes in RNAi, possibly through facilitating processing by dicer.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modifications compared with the specified sequence while retaining essentially same or similar complementary properties as the specified sequence.

### Antisense Strands

The antisense strand of an oligonucleotide may be referred to as a guide strand. For example, if an antisense strand can engage with RNA-induced silencing complex (RISC) and bind to an Argonaute protein, or engage with or bind to one or more similar factors, and direct silencing of a target gene, it may be referred to as a guide strand.

In certain embodiments, the antisense strand is fewer nucleotides in length than the sense strand. In some examples, an oligonucleotide (e.g., RNAi oligonucleotide) provided herein may have an antisense strand including between 10 and 40 nucleotides (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40 nucleotides) in length. Accordingly, the oligonucleotide (e.g., RNAi oligonucleotide) provided herein may have an antisense strand including between 15 and 30 nucleotides (e.g., 15, 16, 17, 19, 20, 21, 22, 23, 24, 25, 26, 27, 28, 29, and 30 nucleotides) in length. For example, the antisense strand may include between 20 and 25 nucleotides (e.g., 20, 21, 22, 23, 24, and 25 nucleotides) in length. In certain embodiments, the antisense strand may be 22 nucleotides in length.

The oligonucleotide disclosed herein may include an antisense strand including a contiguous sequence between 12 and 22 nucleotides (e.g., 12, 13, 14, 15, 16, 17, 18, 19, 20, 21, and 22 nucleotides) in length that is complementary to a sequence of SEQ ID NO: 12. For example, the oligonucleotide may include an antisense strand including a contiguous sequence of between 15 and 21 nucleotides (e.g., 15, 16, 17, 18, 19, 20, and 21 nucleotides) in length that is complementary to a sequence of SEQ ID NOs: 12. In some embodiments, the oligonucleotide may include an antisense strand having a contiguous sequence of 19 nucleotides in length that is complementary to a sequence of SEQ ID NO: 12.

An oligonucleotide disclosed herein may include an antisense strand having a sequence of either of SEQ ID NOS: 3 or 6. In some embodiments, the oligonucleotide disclosed herein may include an antisense strand having the amino acid sequence of SEQ ID NO: 6, as in Compound B shown in FIG. 2B. In some embodiments, the antisense stand may be a pharmaceutically acceptable salt (e.g., a sodium salt) of SEQ ID NO: 6. SEQ ID NO:6 may have the chemical structure as shown in FIG. 1B. Alternatively, the antisense strand may have a sequence of SEQ ID NO: 3, as in Compound A shown in FIGS. 1D and 1E. In some embodiments, the antisense stand may be a pharmaceutically acceptable salt (e.g., a sodium salt) of SEQ ID NO: 3

Additionally, the first position at the 5′ end of antisense strand may be a uridine. The uridine may include a phosphate analog; for example, the uridine may be a 4′-O-monomethylphosphonate-2′-O-methyl uridine.

### Sense Strands

The sense strand of an oligonucleotide may be referred to as a passenger strand. In certain embodiments, the passenger strand is a greater number of nucleotides in length than the guide strand. In some examples, an oligonucleotide (e.g., RNAi oligonucleotide) provided herein may have a sense strand including between 10 and 45 nucleotides (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, and 45 nucleotides) in length. Accordingly, the oligonucleotide (e.g., RNAi oligonucleotide) provided herein may have a sense strand including between 20 and 50 nucleotides (e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 nucleotides) in length. In certain embodiments, the sense strand may be 20 nucleotides in length. In other embodiments, the sense strand may be 36 nucleotides in length.

The oligonucleotide may have a sense strand that includes a contiguous sequence of between 7 to 36 nucleotides in length (e.g., 7, 8, 9, 10, 11, 12, 13, 14, 15, 16, 17, 18, 19, 20, 21, 22, 23, 24, 25, 26, 27, 28, 29, 30, 31, 32, 33, 34, 35, and 36 nucleotides) relative to the sequence of SEQ ID NO: 12. Accordingly, the sense strand may include a contiguous sequence between 10 and 30 nucleotides (e.g., 10, 11, 12, 13, 14, 15, 16, 17, 18, 19, 20, 21, 22, 23, 24, 25, 26, 27, 28, 29, and 30 nucleotides) in length of SEQ ID NO: 12. In some embodiments, the oligonucleotides disclosed herein may include a sense strand that includes a contiguous sequence of nucleotides relative to the sequence of SEQ ID NO: 12 that is 19 nucleotides in length.

The sense strand may include a stem-loop at its 3′-end. In some embodiments, a sense strand includes a stem-loop at its 5′ end. The sense strand including a stem-loop may be in the range of 10 to 50 nucleotides in length (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 nucleotides in length). Accordingly, the sense strand including a stem-loop may be in the range of 20 to 40 nucleotides in length (e.g., 20, 21, 22, 23, 24, 25, 26, 27, 28, 29, 30, 31, 32, 33, 34, 35, 36, 37, 38, 39, and 40 nucleotides in length). For example, the sense strand including a stem-loop may be 36 nucleotides in length.

Furthermore, the stem-loop region on the sense strand may form a duplex region with itself. The duplex region included in the stem-loop may be 1, 2, 3, 4, 5, 6, 7, 8, 9, 10, 11, 12, 13, or 14 nucleotides in length. For example, the duplex region included in the stem-loop may be 6 nucleotides in length. A stem-loop may provide the RNAi oligonucleotide with protection against degradation (e.g., enzymatic degradation) and may facilitate targeting characteristics for delivery to a target cell. For example, a loop may provide added nucleotides on which modification can be made without substantially affecting the gene expression inhibition activity of an oligonucleotide. In certain embodiments, an oligonucleotide provided herein in which the sense strand includes (e.g., at its 3′-end) a stem-loop set forth as: S₁-L-S₂, in which S₁ is complementary to S₂, and in which L forms a loop between S₁ and S₂ of up to 10 nucleotides in length (e.g., 1, 2, 3, 4, 5, 6, 7, 8, 9, or 10 nucleotides in length). Accordingly, the loop between S₁ and S₂ may be 4 nucleotides in length, forming a tetraloop, as described herein. In some embodiments, the S₁ region is 6 nucleotides in length, the S₂ regions is 6 nucleotides in length, and the L region is a 4 nucleotide tetraloop.

The sense strand of the oligonucleotide (e.g., RNAi oligonucleotide) may include a stem-loop region and a region that forms a duplex with the antisense strand. The stem-loop region may include a nucleotide sequence that is at least 85% identical (e.g., 86%, 87%, 88%, 89%, 90%, 91%, 92%, 9%, 94%, 95%, 96%, 97%, 98%, 99%, or more) to the oligonucleotide sequence of SEQ ID NO: 7. In some embodiments, the stem-loop region has the oligonucleotide sequence of SEQ ID NO: 7.

The loop (L) of a stem-loop may be a tetraloop (e.g., within a nicked tetraloop structure). The loop of the stem-loop may have the nucleotide sequence of SEQ ID NO: 8. The tetraloop may contain ribonucleotides, deoxyribonucleotides, modified nucleotides, and combinations thereof. Typically, a loop of a stem-loop has 4 to 5 nucleotides. However, in some embodiments, a loop of a stem-loop may include 3 to 6 nucleotides. For example, the loop of the stem-loop may include 3, 4, 5, or 6 nucleotides. The loop of the stem-loop may include a combination of guanosine and adenosine nucleic acid residues.

An oligonucleotide disclosed herein may include a sense strand sequence having a polynucleotide sequence of any one of SEQ ID NOs: 1, 2, 4, and 5. The sense strand may have a sequence of SEQ ID NO: 4, as in Compound B shown in FIG. 2B. SEQ ID NO: 4 may have the chemical structure as shown in FIG. 1A. In some embodiments, the sense stand may be a pharmaceutically acceptable salt (e.g., a sodium salt) of SEQ ID NO: 4. Alternatively, the sense strand may have a nucleotide sequence of SEQ ID NO: 1, as in Compound A shown in FIGS. 1D and 1E. In some embodiments, the sense stand may be a pharmaceutically acceptable salt (e.g., a sodium salt) of SEQ ID NO: 1.

### Oligonucleotide Modifications

Oligonucleotides may be modified in various ways to improve or control specificity, stability, delivery, bioavailability, resistance from nuclease degradation, immunogenicity, base-paring properties, RNA distribution and cellular uptake and other features relevant to therapeutic or research use, see, Bramsen et al., Nucleic Acids Res., 2009, 37, 2867-2881; Bramsen et al., Frontiers in Genetics, 3 (2012): 1-22). Accordingly, in some embodiments, oligonucleotides of the present disclosure may include one or more suitable modifications. The modified nucleotide may have a modification in its base or nucleobase, the sugar (e.g., ribose, deoxyribose), or the phosphate group.

The number of modifications on an oligonucleotide and the positions of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier, it may be advantageous for at least some of the nucleotides to be modified. Accordingly, in certain embodiments of any of the oligonucleotides provided herein, all, or substantially all, of the nucleotides of an oligonucleotide are modified. In certain embodiments, more than half of the nucleotides are modified. In other embodiments, less than half of the nucleotides are modified. Typically, with naked delivery, every sugar is modified at the 2′-position. These modifications may be reversible or irreversible. The oligonucleotide as disclosed herein may have a number and type of modified nucleotides sufficient to cause the desired characteristic (e.g., protection from enzymatic degradation, capacity to target a desired cell after in vivo administration, and/or thermodynamic stability).

### Sugar Modifications

A modified sugar, also referred herein to a sugar analog, includes a modified deoxyribose or ribose moiety, in which one or more modifications occur at the 2′,3′,4′, and/or 5′ carbon position of the sugar. The modified sugar may also include non-natural alternative carbon structures such as those present in locked nucleic acids (“LNA”) (see, Koshkin et al. (1998), Tetrahedron 54, 3607-3630), unlocked nucleic acids (“UNA”) (see, Snead et al. (2013), Molecular Therapy-Nucleic Acids, 2, e103), and bridged nucleic acids (“BNA”) (see, Imanishi and Obika (2002), The Royal Society of Chemistry, Chem. Commun., 1653-1659). Koshkin et al., Snead et al., and Imanishi and Obika are incorporated by reference herein for their disclosures relating to sugar modifications.

A nucleotide modification at a sugar may include a 2′-modification. A 2′-modification may be 2′-aminoethyl, 2′-fluoro, 2′-O-methyl, 2′-O-methoxyethyl, and 2′-deoxy-2′-fluoro-β-d-arabinonucleic acid. Typically, the modification is 2′-fluoro, 2′-O-methyl, or 2′-O-methoxyethyl. In some embodiments, the modification is a 2′-fluoro and/or a 2′-O-methyl. In some embodiments, the 2′-fluoro modification is 2′-fluoro deoxyribonucleoside and/or the 2′-O-methyl modification is 2′-O-methyl ribonucleoside. A modification at a sugar may include a modification of the sugar ring, which may have a modification of one or more carbons of the sugar ring. For example, a modification of a sugar of a nucleotide may include a 2′-oxygen of a sugar linked to a 1′-carbon or 4′-carbon of the sugar, or a 2′-oxygen linked to the 1′-carbon or 4′-carbon via an ethylene or methylene bridge. In certain embodiments, a modified nucleotide may have an acyclic sugar that lacks a 2′-carbon to 3′-carbon bond. In some embodiments, a modified nucleotide may have a thiol group, e.g., in the 4′ position of the sugar.

The oligonucleotide (e.g., RNAi oligonucleotide) described herein may include at least one modified nucleotide (e.g., at least 1, at least 5, at least 10, at least 15, at least 20, at least 25, at least 30, at least 35, at least 40, at least 45, at least 50, at least 55, at least 60, or more). For example, the sense strand of the oligonucleotide may include at least one modified nucleotide (e.g., at least 1, at least 5, at least 10, at least 15, at least 20, at least 25, at least 30, at least 35, or more). Also, for example, the antisense strand of the oligonucleotide may include at least one modified nucleotide (e.g., at least 1, at least 5, at least 10, at least 15, at least 20, or more).

In certain embodiments, the oligonucleotide (e.g., RNAi oligonucleotide) described herein may contain between 20 and 50 (e.g., 20 to 30, 24 to 30, 28 to 30, 30 to 40, 34 to 40, 38 to 44, 44 to 50, and 48 to 50) modified nucleotides.

All of the nucleotides of the sense strand of the oligonucleotide may be modified. Furthermore, all of the nucleotides of the antisense strand of the oligonucleotide may be modified. In some embodiments, all of the nucleotides of the oligonucleotide (e.g., RNAi oligonucleotide) including both the sense strand and the antisense strand are modified. The modified nucleotide may be a 2′-modification (e.g., a 2′-fluoro or 2′-O-methyl). The 2′-modification to the nucleotide may be a 2′-fluoro and/or a 2′-O-methyl, wherein optionally the 2′-fluoro modification is 2′-fluoro deoxyribonucleoside and/or the 2′-O-methyl modification is 2′-O-methyl ribonucleoside.

The disclosure provides oligonucleotides having different modification patterns. The oligonucleotide including the sense strand and the antisense strand may include between 40 and 50 (e.g., 41, 2, 43, 44, 45, 46, 47, 48, and 49) 2′-O-methyl modifications. The modified oligonucleotides may include a sense strand having a nucleotide sequence of either of SEQ ID NO: 1 or 4, and an antisense strand having a nucleotide sequence of either of SEQ ID NO: 3 or 6 (e.g., the RNAi oligonucleotide may have a sense strand of SEQ ID NO: 4 and an antisense strand of SEQ ID NO: 6, or the RNAi oligonucleotide may have a sense strand of SEQ ID NO: 1 and an antisense strand of SEQ ID NO: 3). In some embodiments, for these oligonucleotides, one or more of positions 1, 2, 3, 4, 5, 6, 7, 11, 12, 13, 14, 15, 16, 17, 18, 19, 20, 21, 22, 23, 24, 25, 26, 27, 31, 32, 33, 34, 35, and 36 of the sense strand, and/or one or more of positions 1, 6, 8, 9, 11, 12, 13, 15, 16, 17, 18, 19, 20, 21, and 22 of the antisense strand are modified with a 2′-O-methyl modified nucleoside, such as a 2′-O-methyl ribonucleoside. In some embodiments, all of positions 1, 2, 3, 4, 5, 6, 7, 12, 13, 14, 15, 16, 17, 18, 19, 20, 21, 22, 23, 24, 25, 26, 27, 31, 32, 33, 34, 35, and 36 of the sense strand, and all of positions 1, 6, 8, 9, 11, 12, 13, 15, 16, 17, 18, 19, 20, 21, and 22 of the antisense strand are modified with a 2′-O-methyl modified nucleoside, such as a 2′-O-methyl ribonucleoside. In other embodiments, one or more of positions 1, 2, 4, 5, 6, 7, 11, 14, 15, 16, 18, 19, 20, 21, 22, 23, 24, 25, 26, 27, 31, 32, 33, 34, 35, and 36 of the sense strand, and/or one or more of positions 1, 6, 9, 11, 13, 15, 17, 18, 20, 21, and 22 of the antisense strand are modified with a 2′-O-methyl modified nucleoside, such as a 2′-O-methyl ribonucleoside. In certain embodiments, all of positions 1, 2, 4, 5, 6, 7, 11, 14, 15, 16, 18, 19, 20, 21, 22, 23, 24, 25, 26, 27, 31, 32, 33, 34, 35, and 36 of the sense strand, and/or all of positions 1, 6, 9, 11, 13, 15, 17, 18, 20, 21, and 22 of the antisense strand are modified with a 2′-O-methyl modified nucleoside, such as a 2′-O-methyl ribonucleoside.

The oligonucleotide including the sense strand and the antisense strand may have between 5 and 15 (e.g., 6, 7, 8, 9, 10, 11, 12, 13, and 14) 2′-fluoro modifications. For these oligonucleotides, one or more of positions 8, 9, 10, 11, 12, 13, and 17 of the sense strand, and/or one or more of positions 2, 3, 4, 5, 7, 10, 14, 16, and 19 of the antisense strand may be modified with a 2′-fluoro modified nucleoside. For example, all of positions 8, 9, 10, and 11 of the sense strand, and/or all of positions 2, 3, 4, 5, 7, 10, and 14 of the antisense strand may be modified with a 2′-fluoro modified nucleoside. In other embodiments, one or more of positions 3, 8, 10, 12, 13, and 17 of the sense strand, and/or one or more of positions 2, 3, 4, 5, 7, 8, 10, 12, 14, 16, and 19 of the antisense strand may be modified. In another example, all of positions 3, 8, 9, 10, 12, 13, and 17 of the sense strand, and/or all of positions 2, 3, 4, 5, 7, 8, 10, 12, 14, 16, and 19 of the antisense strand may be modified with a 2′-fluoro modified nucleoside.

For oligonucleotides comprising a sense strand having a sequence of SEQ ID NO: 1, and an antisense strand having a sequence of SEQ ID NO: 3, one or more of positions 1-7, 12-27, and 31-36 of the sense strand, and/or one or more of positions 1, 6, 8, 9, 11-13, and 15-22 of the antisense strand may be modified with a 2′-O-methyl modified nucleoside. Furthermore, all of positions 1-7, 12-27, and 31-36 of the sense strand, and/or one or more of positions 1, 6, 8, 9, 11-13, and 15-22 of the antisense strand may be modified with a 2′-O-methyl modified nucleoside. For oligonucleotides with a sense strand having a sequence of SEQ ID NO: 1, and an antisense strand having sequence of SEQ ID NO: 3, one or more of positions 8-11 of the sense strand, and one or more of positions 2, 3, 4, 5, 7, 10, and 14 of the antisense strand may be modified with a 2′-fluoro modified nucleoside. Accordingly, all of positions 8-11 of the sense strand, and all of positions 2, 3, 4, 5, 7, 10, and 14 of the antisense strand may be modified with a 2′-fluoro modified nucleoside.

For example, for oligonucleotides with a sense strand having a sequence of SEQ ID NO: 1, and an antisense strand having a sequence of SEQ ID NO: 3, all of positions 1-7, 12-27, and 31-36 of the sense strand, and/or one or more of positions 1, 6, 8, 9, 11-13, and 15-22 of the antisense strand may be modified with a 2′-O-methyl modified nucleoside; and all of positions 8-11 of the sense strand, and all of positions 2, 3, 4, 5, 7, 10, and 14 of the antisense strand may be modified with a 2′-fluoro, where the chemical structure of the sense strand is shown in FIG. 1A, the antisense strand is shown in FIG. 1B, and the RNAi oligonucleotide is shown in FIG. 1C-1 and FIG. 1C-2.

For oligonucleotides comprising a sense strand having a sequence of SEQ ID NO: 4, and an antisense strand having a sequence of SEQ ID NO: 6, one or more of positions 1, 2, 4, 5, 6, 7, 11, 14, 15, 16, 18, 19, 20, 21, 22, 23, 24, 25, 26, 27, 31, 32, 33, 34, 35, and 36 of the sense strand, and/or one or more of positions 1, 6, 9, 11, 13, 15, 17, 18, 20, 21, and 22 of the antisense strand may be modified with a 2′-O-methyl. In some embodiments, all of positions 1, 2, 4, 5, 6, 7, 11, 14, 15, 16, 18, 19, 20, 21, 22, 23, 24, 25, 26, 27, 31, 32, 33, 34, 35, and 36 of the sense strand, and all of positions 1, 6, 9, 11, 13, 15, 17, 18, 20, 21, and 22 of the antisense strand may be modified with a 2′-O-methyl. Additionally for oligonucleotides having a sense strand having a sequence of SEQ ID NO: 4, and an antisense strand having a sequence of SEQ ID NO: 6, one or more of positions 3, 8, 9, 10, 12, 13, and 17 of the sense strand, and/or one or more of positions 2, 3, 4, 5, 7, 8, 10, 12, 14, 16, and 19 of the antisense strand may be modified with a 2′-fluoro. In some embodiments, all of positions 3, 8, 9, 10, 12, 13, and 17 of the sense strand, and all of positions 2, 3, 4, 5, 7, 8, 10, 12, 14, 16, and 19 of the antisense strand are modified with a 2′-fluoro. For example, for oligonucleotides having a sense strand including a sequence of SEQ ID NO: 4 and an antisense strand having a sequence of SEQ ID NO: 6, all of positions 1, 2, 4, 5, 6, 7, 11, 14, 15, 16, 18, 19, 20, 21, 22, 23, 24, 25, 26, 27, 31, 32, 33, 34, 35, and 36 of the sense strand, and all of positions 1, 6, 9, 11, 13, 15, 17, 18, 20, 21, and 22 of the antisense strand may be modified with a 2′-O-methyl; and all of positions 3, 8, 9, 10, 12, 13, and 17 of the sense strand, and all of positions 2, 3, 4, 5, 7, 8, 10, 12, 14, 16, and 19 of the antisense strand may be modified with a 2′-fluoro; the chemical structures of the sense strand and the antisense strand are shown in FIG. 2A-1 and FIG. 2A-2.

In some embodiments, the terminal 3′-end group (e.g., a 3′-hydroxyl) may be modified with a phosphate group or other group, which can be used, for example, to attach linkers, adapters, or labels or for the direct ligation of an oligonucleotide to another nucleic acid.

### 5′ Terminal Phosphates

The 5′-terminal phosphate groups of the oligonucleotide (e.g., RNAi oligonucleotide) may enhance the interaction with Argonaute 2. In certain embodiments, the oligonucleotide (e.g., RNAi oligonucleotide) includes a uridine at the first position of the 5′ end of the antisense strand. However, oligonucleotides having a 5′-phosphate group may be susceptible to degradation via phosphatases or other enzymes, which can limit their bioavailability in vivo. In some embodiments, oligonucleotides include analogs of 5′ phosphates that are resistant to such degradation. Therefore, the uridine at the 5′ end of the antisense strand may include a phosphate analog. The phosphate analog may be oxymethylphosphonate, vinylphosphonate, or malonylphosphonate. Furthermore, the 5′ end of an oligonucleotide strand may be attached to a chemical moiety that mimics the electrostatic and steric properties of a natural 5′-phosphate group (“phosphate mimic”) (see, Prakash et al., Nucleic Acids Res. 2015 Mar. 31; 43 (6): 2993-3011, the contents of which relating to phosphate analogs are incorporated herein by reference). Many phosphate mimics have been developed that can be attached to the 5′ end (see, U.S. Pat. No. 8,927,513, the contents of which relating to phosphate analogs are incorporated herein by reference). Other modifications have been developed for the 5′ end of oligonucleotides (see, WO 2011/133871, the contents of which relating to phosphate analogs are incorporated herein by reference). In certain embodiments, a hydroxyl group may be attached to the 5′ end of the oligonucleotide.

The oligonucleotide may have a phosphate analog at a 4′-carbon position of the sugar, referred to as a “4′-phosphate analog”. See, for example, WO 2018/045317, the contents of which relating to phosphate analogs are incorporated herein by reference. The oligonucleotide provided herein may include a 4′-phosphate analog at a 5′-terminal nucleotide. In some embodiments, the phosphate analog is an oxymethylphosphonate, in which the oxygen atom of the oxymethyl group is bound to the sugar moiety (e.g., at its 4′-carbon) or analog thereof. In other embodiments, a 4′-phosphate analog is a thiomethylphosphonate or an aminomethylphosphonate, in which the sulfur atom of the thiomethyl group or the nitrogen atom of the aminomethyl group is bound to the 4′-carbon of the sugar moiety or analog thereof. In certain embodiments, a 4′-phosphate analog is an oxymethylphosphonate. In some embodiments, an oxymethylphosphonate is represented by the formula —O—CH₂—PO(OH)₂ or —O—CH₂—PO(OR)₂, in which R is independently selected from H, CH₃, an alkyl group, CH₂CH₂CN, CH₂OCOC(CH₃)₃, CH₂OCH₂CH₂Si(CH₃)₃, or a protecting group. In certain embodiments, the alkyl group is CH₂CH₃. More typically, R is independently selected from H, CH₃, or CH₂CH₃. In some embodiments, R is CH₃. In some embodiments, the 4′-phosphate analog is 5′-methoxyphosphanate-4′-oxy. In some embodiments, the 4′-phosphate analog is 4′-(methyl methoxyphosphonate). In some embodiments, the phosphate analog is a 4′-O-monomethylphosphonate analog.

In some embodiments, a phosphate analog attached to the oligonucleotide is a methoxy phosphonate (MOP). The phosphate analog attached to the oligonucleotide may be a 5′ monomethyl protected MOP. In some embodiments, the following uridine nucleotide comprising a phosphate analog may be used, e.g., at the first position of the antisense strand:

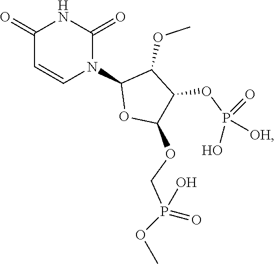 [SMILES: CO[C@@H]1[C@H](OP(=O)(O)O)[C@@H](OCP(=O)(O)OC)O[C@H]1n1ccc(=O)[nH]c1=O]

which modified nucleotide is referred to as [MePhosphonate-4O-mU] or 5′-Methoxy, Phosphonate-4′oxy-2′-O-methyluridine. The 5′-Methoxy, Phosphonate-4′oxy-2′-O-methyluridine may be the first nucleotide at the 5′ end of the antisense strand. For example, the first nucleotide at the 5′ end of either of SEQ ID NOs: 3 or 6 may be a 5′-Methoxy, Phosphonate-4′oxy-2′-O-methyluridine.

### Modified Internucleoside Linkages

Phosphate modifications or substitutions in the oligonucleotide may result in an oligonucleotide that includes at least one (e.g., at least 1, at least 2, at least 3, at least 5, or at least 6) modified internucleotide linkage. Any one of the oligonucleotides disclosed herein may include between 1 and 10 (e.g., 1 to 10, 2 to 8, 4 to 6, 3 to 10, 5 to 10, 1 to 5, 1 to 3 or 1 to 2) modified internucleotide linkages. For example, any one of the oligonucleotides disclosed herein may include 1, 2, 3, 4, 5, 6, 7, 8, 9, or 10 modified internucleotide linkages. In some embodiments, the oligonucleotide (e.g., RNAi oligonucleotide) may include 5 modified internucleotide linkages. For example, the sense stand of the oligonucleotide may include 1 modified internucleotide linkage, and the antisense strand may include 4 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. At least one modified internucleotide linkage of any one of the oligonucleotides as disclosed herein may be a phosphorothioate linkage. In certain embodiments, all of the modified internucleotide linkages of the oligonucleotide may be phosphorothioate linkages.

The oligonucleotide described herein may have a phosphorothioate linkage between one or more of: positions 1 and 2 of the sense strand, positions 1 and 2 of the antisense strand, positions 2 and 3 of the antisense strand, positions 20 and 21 of the antisense strand, and positions 21 and 22 of the antisense strand. For example, the sense strand of the oligonucleotide may have a phosphorothioate linkage between positions 1 and 2 of the sense strand, positions 1 and 2 of the antisense strand, positions 2 and 3 of the antisense strand, positions 20 and 21 of the antisense strand, and positions 21 and 22 of the antisense strand. Accordingly, the sense strand having a sequence of SEQ ID NO: 1 or 4 may have a phosphorothioate linkage between positions 1 and 2, and the antisense strand having a sequence of SEQ ID NO: 3 or 6 may have a phosphorothioate linkage between positions 1 and 2, 2 and 3, 20 and 21, and 21 and 22.

### Base Modifications

The oligonucleotides provided herein may have one or more modified nucleobases. Modified nucleobases, also referred to herein as base analogs, may be linked at the 1′ position of a nucleotide sugar moiety. The modified nucleobase may be a nitrogenous base. In certain embodiments, the modified nucleobase may contain a nitrogen atom. See, U.S. Published Patent Application No. 2008/0274462 the contents of which relating to modified nucleobases are incorporated herein by reference. The modified nucleotide may also include a universal base. However, in certain embodiments, a modified nucleotide may not contain a nucleobase (e.g., abasic).

In some embodiments a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering the structure of the duplex. In some embodiments, compared to a reference single-stranded nucleic acid (e.g., oligonucleotide or polynucleotide) that is fully complementary to a target nucleic acid, a single-stranded nucleic acid containing a universal base forms a duplex with the target nucleic acid that has a lower Tm than a duplex formed with the complementary nucleic acid. However, in some embodiments, compared to a reference single-stranded nucleic acid in which the universal base has been replaced with a base to generate a single mismatch, the single-stranded nucleic acid containing the universal base forms a duplex with the target nucleic acid that has a higher Tm than a duplex formed with the nucleic acid comprising the mismatched base. Non-limiting examples of universal-binding nucleotides include inosine, 1-β-D-ribofuranosyl-5-nitroindole, and/or 1-β-D-ribofuranosyl-3-nitropyrrole (see, US 2007/0254362; Van Aerschot et al., Nucleic Acids Res. 1995 Nov. 11; 23 (21): 4363-70; Loakes et al., Nucleic Acids Res. 1995 Jul. 11; 23 (13): 2361-6; Loakes et al., Nucleic Acids Res. 1994 Oct. 11; 22 (20): 4039-43. Each of the foregoing is incorporated by reference herein for their disclosures relating to base modifications).

### Reversible Modifications

While certain modifications to protect an oligonucleotide from the in vivo environment before reaching target cells can be made, they can reduce the potency or activity of the oligonucleotide once it reaches the cytosol of the target cell. Reversible modifications can be made such that the molecule retains desirable properties outside of the cell, which are then removed upon entering the cytosolic environment of the cell. Reversible modification can be removed, for example, by the action of an intracellular enzyme or by the chemical conditions inside of a cell (e.g., through reduction by intracellular glutathione).

A reversibly modified nucleotide may include a glutathione-sensitive moiety. Typically, nucleic acid molecules may be chemically modified with cyclic disulfide moieties to mask the negative charge created by the internucleotide diphosphate linkages and improve cellular uptake and nuclease resistance. See US 2011/0294869 originally assigned to Traversa Therapeutics, Inc. (“Traversa”), PCT Publication No. WO 2015/188197 to Solstice Biologics, Ltd. (“Solstice”), Meade et al., Nature Biotechnology, 2014,32:1256-1263 (“Meade”), PCT Publication No. WO 2014/088920 to Merck Sharp & Dohme Corp, each of which are incorporated by reference for their disclosures of such modifications. The reversible modification of the internucleotide diphosphate linkages is designed to be cleaved intracellularly by the reducing environment of the cytosol (e.g., glutathione). Earlier examples include neutralizing phosphotriester modifications that were reported to be cleavable inside cells (see, Dellinger et al. J. Am. Chem. Soc. 2003, 125:940-950).

Such a reversible modification allows protection during in vivo administration (e.g., transit through the blood and/or lysosomal/endosomal compartments of a cell) where the oligonucleotide will be exposed to nucleases and other harsh environmental conditions (e.g., pH). When released into the cytosol of a cell where the levels of glutathione are higher compared to extracellular space, the modification is reversed, and the result is a cleaved oligonucleotide. Using reversible, glutathione sensitive moieties, it is possible to introduce sterically larger chemical groups into the oligonucleotide of interest as compared to the options available using irreversible chemical modifications. This is because these larger chemical groups will be removed in the cytosol and, therefore, should not interfere with the biological activity of the oligonucleotides inside the cytosol of a cell. As a result, these larger chemical groups can be engineered to confer various advantages to the nucleotide or oligonucleotide, such as nuclease resistance, lipophilicity, charge, thermal stability, specificity, and reduced immunogenicity. The structure of the glutathione-sensitive moiety may be engineered to modify the kinetics of its release.

In some embodiments, a glutathione-sensitive moiety is attached to the sugar of the nucleotide. In some embodiments, a glutathione-sensitive moiety is attached to the 2′carbon of the sugar of a modified nucleotide. In some embodiments, the glutathione-sensitive moiety is located at the 5′-carbon of a sugar, for example when the modified nucleotide is the 5′-terminal nucleotide of the oligonucleotide. In some embodiments, the glutathione-sensitive moiety is located at the 3′-carbon of sugar, for example when the modified nucleotide is the 3′-terminal nucleotide of the oligonucleotide. In some embodiments, the glutathione-sensitive moiety comprises a sulfonyl group. See, e.g., U.S. Publication Application number 2019/0177355, the contents of which are incorporated by reference herein for its relevant disclosures.

### Targeting Ligands

It may be desirable to target the oligonucleotides of the disclosure to one or more cells or one or more organs (e.g., cells of the liver). Such a strategy may help to avoid undesirable effects in other organs or may avoid undue loss of the oligonucleotide to cells, tissue or organs that would not benefit for the oligonucleotide. Accordingly, in some embodiments, oligonucleotides disclosed herein may be modified to facilitate targeting of a particular tissue, cell, or organ, e.g., to facilitate delivery of the oligonucleotide to the liver. In certain embodiments, oligonucleotides disclosed herein may be modified to facilitate delivery of the oligonucleotide to the hepatocytes of the liver. An oligonucleotide may include a nucleotide that is conjugated to one or more targeting ligand.

A targeting ligand may include a carbohydrate, amino sugar, cholesterol, peptide, polypeptide, protein, or part of a protein (e.g., an antibody or antibody fragment) or lipid. In some embodiments, a targeting ligand is an aptamer. For example, a targeting ligand may be an RGD peptide that is used to target tumor vasculature or glioma cells, CREKA peptide to target tumor vasculature or stoma, transferring, lactoferrin, or an aptamer to target transferrin receptors expressed on CNS vasculature, or an anti-EGFR antibody to target EGFR on glioma cells. In some embodiments, the targeting ligand is one or more N-Acetylgalactosamine (GalNAc) moieties.

One or more (e.g., 1, 2, 3, 4, 5 or 6) nucleotides of an oligonucleotide may be each conjugated to a separate targeting ligand. In some instances, 2 to 4 nucleotides of an oligonucleotide are each conjugated to a separate targeting ligand. The targeting ligands may be conjugated to 2 to 4 nucleotides at either ends of the sense or antisense strand (e.g., the ligand is conjugated to a 2 to 4 nucleotide overhang or extension on the 5′ or 3′ end of the sense or antisense strand) such that the targeting ligands resemble bristles of a toothbrush and the oligonucleotide resembles a toothbrush. For example, an oligonucleotide may include a stem-loop at either the 5′ or 3′ end of the sense strand and 1, 2, 3, or 4 nucleotides of the loop of the stem may be individually conjugated to a targeting ligand. In some embodiments, the oligonucleotide includes a stem-loop at the 3′ end of the sense strand and 3 nucleotides of the loop of the stem are individually conjugated to a targeting ligand.

In some embodiments, it is desirable to target an oligonucleotide that reduces the expression of C3 to the hepatocytes of the liver of the subject. Any suitable hepatocyte targeting moiety may be used for this purpose.

GalNAc is a high affinity ligand for asialoglycoprotein receptors (ASGPR), which are primarily expressed on the sinusoidal surface of hepatocyte cells and has a major role in binding, internalization, and subsequent clearance of circulating glycoproteins that contain terminal galactose or N-acetylgalactosamine residues (asialoglycoproteins). Conjugation, either indirect or direct, of GalNAc moieties to oligonucleotides of the instant disclosure may be used to target these oligonucleotides to the ASGPR expressed on these hepatocyte cells.

For example, an oligonucleotide of the disclosure may be conjugated directly or indirectly to a monovalent GalNAc. The oligonucleotide may be conjugated directly or indirectly to more than one (e.g., 2, 3, 4, or more) monovalent GalNAc, and is typically conjugated to 3 or 4 monovalent GalNAc moieties. The GalNAc moiety (ies) may be present within a loop region of the oligonucleotides described herein. The GalNAc moiety may be used to target the oligonucleotides of the disclosure to ASGPR on hepatocytes; at which point, the GalNAc conjugated oligonucleotide may be internalized and integrated into the intracellular RNAi machinery called the RNA-induced silencing complex (RISC). The RISC Argonaute-2 (Argo-2) protein within this complex targets the antisense strand of the oligonucleotide duplex to its complementary C3 mRNA and initiates its degradation, thus blocking translation of the target.

In some embodiments, 2 to 4 nucleotides of the loop (L) of the stem-loop are each conjugated to a separate GalNAc moiety. In some embodiments, three nucleotides of the loop of the stem-of the oligonucleotide may be conjugated directly or indirectly to three separate monovalent GalNAc moieties. In some embodiments, the oligonucleotide is conjugated to one or more bivalent GalNAc, trivalent GalNAc, or tetravalent GalNAc moieties.

The oligonucleotide described herein may include a monovalent GalNAc attached to a guanine nucleobase, referred to as [ademG-GalNAc] or 2′-aminodiethoxymethanol-guanine-GalNAc, as depicted below:

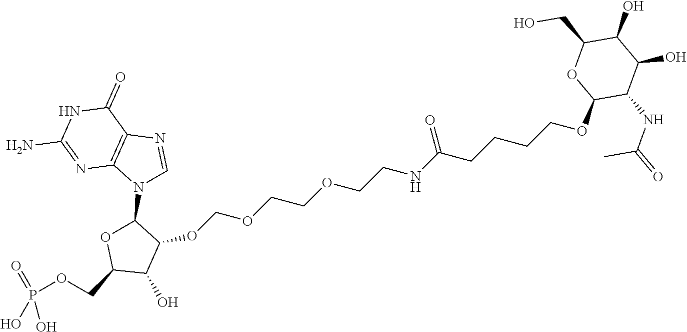 [SMILES: CC(=O)N[C@@H]1[C@@H](OCCCCC(=O)NCCOCCOCO[C@@H]2[C@H](O)[C@@H](COP(=O)(O)O)O[C@H]2n2cnc3c(=O)[nH]c(N)nc32)O[C@@H](CO)[C@@H](O)[C@H]1O]

Additionally, or alternatively, the oligonucleotide herein may include a monovalent GalNAc attached to an adenine nucleobase, referred to as [ademA-GalNAc] or 2′-aminodiethoxymethanol-adenine-GalNAc, as depicted below.

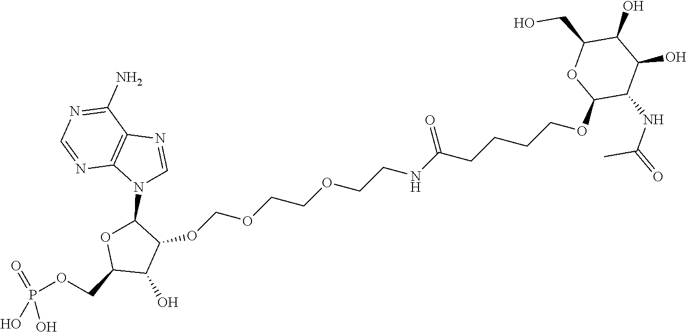

An example of such conjugation is shown below for a loop comprising from 5′ to 3′ the nucleotide sequence GAAA (SEQ ID NO: 8) (L=linker, X=heteroatom) stem attachment points are shown. Such a loop may be present, for example, at nucleotide positions 27-30 of the molecule shown in FIG. 1A. In the chemical formula,

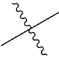

is an attachment point to the oligonucleotide stand.

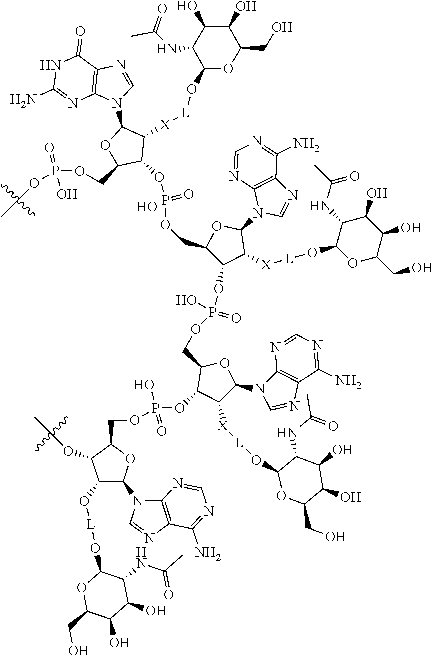

Appropriate methods or chemistry (e.g., click chemistry) can be used to link a targeting ligand to a nucleotide. A targeting ligand may be conjugated to a nucleotide using a click linker. Furthermore, an acetal-based linker may be used to conjugate a targeting ligand to a nucleotide of any one of the oligonucleotides described herein. Acetal-based linkers are disclosed, for example, in International Patent Application Publication Number WO 2016/100401 A1, which published on Jun. 23, 2016, and the contents of which relating to such linkers are incorporated herein by reference. The linker may be a labile linker. However, in other embodiments, the linker is stable (non-labile).

An example is shown below for a tetraloop comprising from 5′ to 3′ the nucleotides GAAA (SEQ ID NO: 8), in which four (4) GalNAc moieties are attached to nucleotides of the loop using an acetal linker. Such a loop may be present in an oligonucleotide disclosed herein (see, for example, positions 27-30 of the oligonucleotides having the sequences of SEQ ID NOs: 1 and 4). In the chemical formula,

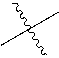

is an attachment point to the oligonucleotide strand.

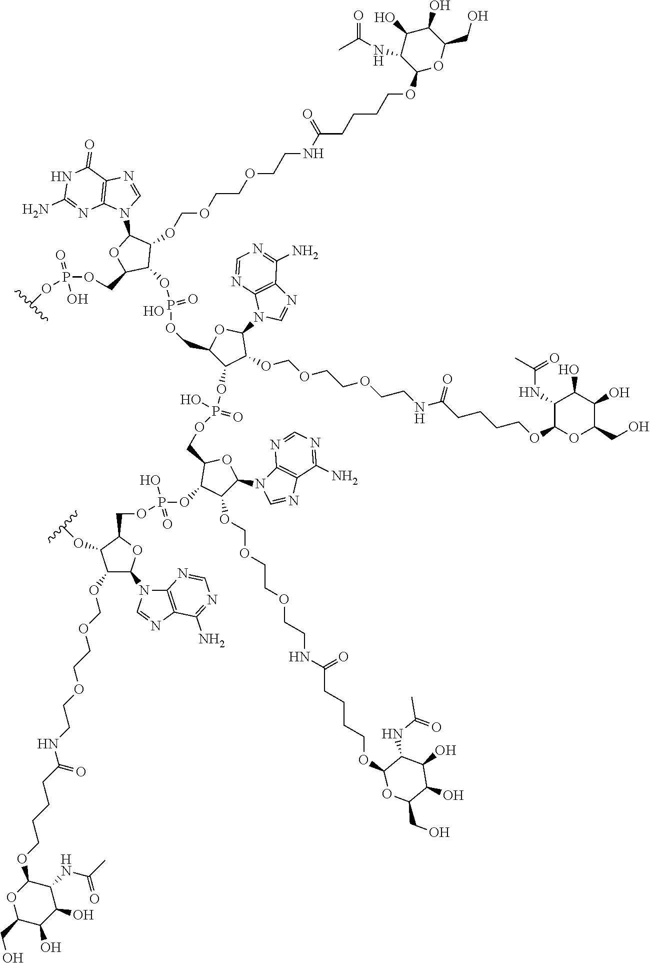 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

In some embodiments, an oligonucleotide herein (e.g., an RNAi oligonucleotide) comprises a sense strand having a tetraloop, wherein three (3) GalNAc moieties are conjugated to nucleotides comprising the tetraloop, and wherein each GalNAc moiety is conjugated to one (1) nucleotide. In some embodiments, an oligonucleotide herein (e.g., an RNAi oligonucleotide) comprises a sense strand having a tetraloop comprising GalINAc-conjugated nucleotides, wherein the tetraloop comprises the following structure:

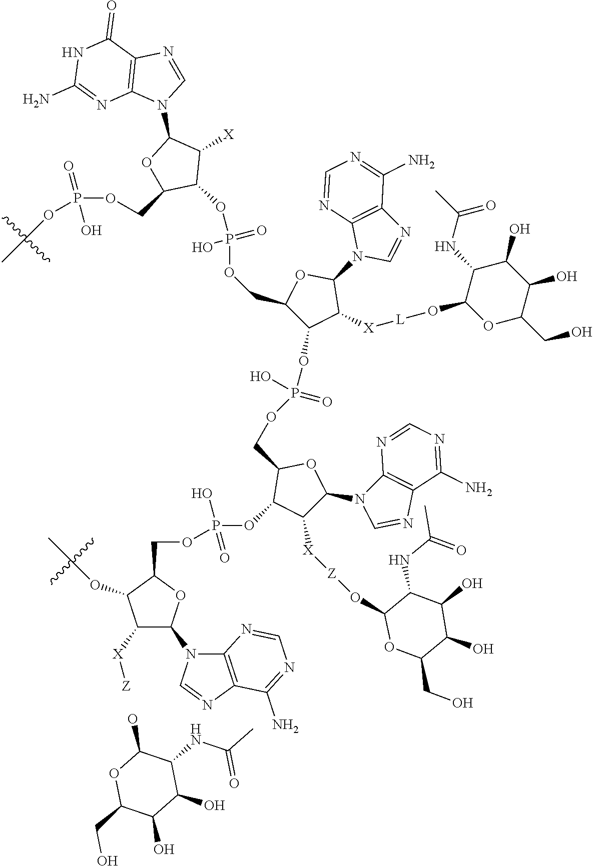

in which:
Z represents a bond, click chemistry handle, or a linker of 1 to 20, inclusive, consecutive, covalently bonded atoms in length, selected from the group consisting of substituted and unsubstituted alkylene, substituted and unsubstituted alkenylene, substituted and unsubstituted alkynylene, substituted and unsubstituted heteroalkylene, substituted and unsubstituted heteroalkenylene, substituted and unsubstituted heteroalkynylene, and combinations thereof; and
X is an O, S, or N.

In another embodiment, an oligonucleotide herein (e.g., an RNAi oligonucleotide) comprises a sense strand having a tetraloop comprising three (3) GalNAc moieties conjugated to nucleotides, wherein the tetraloop comprises the following structure:

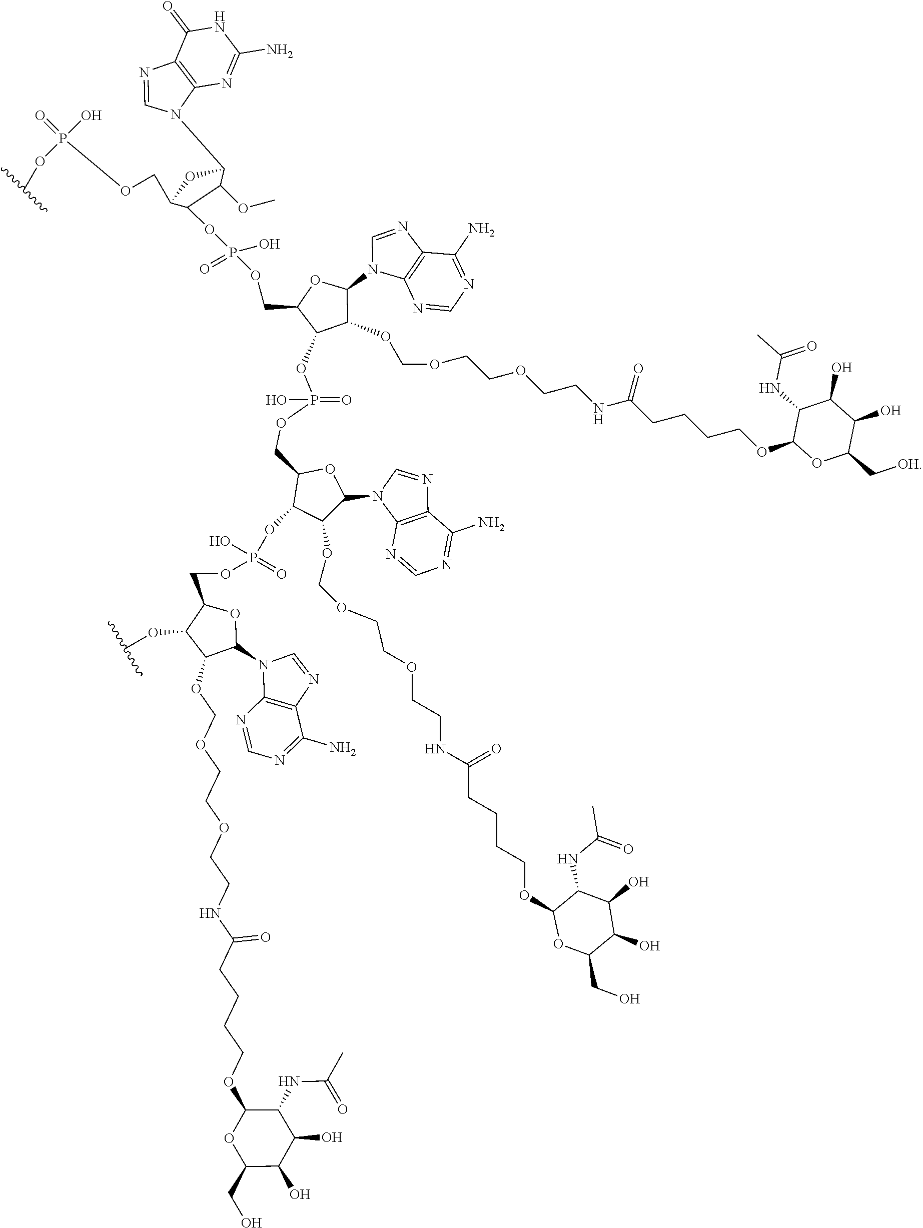

In some embodiments, a duplex extension (e.g., of up to 3, 4, 5, or 6 base pairs in length) is provided between a targeting ligand (e.g., a GalNAc moiety) and an oligonucleotide (e.g., RNAi oligonucleotide). In some embodiments, the duplex extension between a targeting ligand (e.g., a GalNAc moiety) and an oligonucleotide (e.g., RNAi oligonucleotide) is 6 base pairs in length.

### Formulations

Various formulations have been developed to facilitate oligonucleotide use. For example, oligonucleotides can be delivered to a subject or a cellular environment using a formulation that minimizes degradation, facilitates delivery and/or uptake, or provides another beneficial property to the oligonucleotides in the formulation. In some embodiments, provided herein are compositions including oligonucleotides (e.g., single-stranded or double-stranded oligonucleotides) to reduce the expression of C3. Such compositions can be suitably formulated such that when administered to a subject, either into the immediate environment of a target cell or systemically, a sufficient portion of the oligonucleotides enter the cell to reduce C3 expression. Any of a variety of suitable oligonucleotide formulations can be used to deliver oligonucleotides for the reduction of C3 as disclosed herein. In some embodiments, an oligonucleotide, the pharmaceutical composition, the vector, or the cell is formulated in buffer solutions such as phosphate buffered saline solutions, liposomes, micellar structures, vectors, and capsids.

Formulations as disclosed herein may include an excipient. The excipient may confer to a composition improved stability, improved absorption, improved solubility, and/or therapeutic enhancement of the active ingredient. The excipient may be a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide may be lyophilized for extending its shelf-life and then made into a solution before use (e.g., administration to a subject). Accordingly, an excipient in a composition including any one of the oligonucleotides described herein may be a lyoprotectant (e.g., mannitol, lactose, polyethylene glycol, or polyvinyl pyrolidone), or a collapse temperature modifier (e.g., dextran, ficoll, or gelatin).

The pharmaceutical composition including the oligonucleotide may be formulated to be compatible with its intended route of administration. Examples of routes of administration include parenteral, e.g., subcutaneous, intravenous, intradermal, oral (e.g., inhalation), transdermal (topical), transmucosal, and rectal administration (e.g., subcutaneous administration).

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.), or phosphate buffered saline (PBS). The carrier can be a solvent or dispersion medium containing, for example, water, ethanol, polyol (e.g., glycerol, propylene glycol, liquid polyetheylene glycol, and the like), and suitable mixtures thereof. In many cases, it will be optional to include isotonic agents, for example, sugars, polyalcohols, such as mannitol and sorbitol, and sodium chloride in the composition. Sterile injectable solutions may be prepared by incorporating the oligonucleotides in a required amount in a selected solvent with one or a combination of ingredients enumerated above, as required, followed by filtered sterilization.

In some embodiments, a pharmaceutical composition including the oligonucleotide comprises sterile water (or water for injection (WFI)). In some embodiments, a pharmaceutical composition including the oligonucleotide comprises PBS.

In some embodiments, a pharmaceutical composition including the oligonucleotide includes a preservative-free, sterile solution in WFI. In some embodiments, the pH of pharmaceutical composition is about 7.2 (e.g., pH 7.2). In some embodiments, 0.1N NaOH or 0.1N HCl may be titrated if necessary to adjust the pH of the solution to a target of 7.2. In some embodiments, the concentration of free acid form of the RNAi oligonucleotide in the pharmaceutical composition is about 160 mg/ml (e.g., 160 mg/mL). The WFI may be used in some embodiments to bring the total concentration to about 160 mg/mL as the free acid form. In some embodiments, the target fill volume is about 1.3 mL into a 2-mL glass vial. In some embodiments, the solution is expected to be given to patients subcutaneously as its route of administration.

In some embodiments, a composition may contain at least about 0.1% of the therapeutic agent (e.g., an oligonucleotide for reducing C3 expression) or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Even though a number of embodiments are directed to liver-targeted delivery of any of the oligonucleotides disclosed herein, targeting of other tissues is also contemplated.

### Pharmaceutical Use

Disclosed herein are methods for delivering to a cell or a subject an effective amount of any one of the oligonucleotides (e.g., RNAi oligonucleotides) disclosed herein for purposes of reducing expression of C3 in the cell or subject.

The oligonucleotides disclosed herein can be introduced to a cell of a subject with a disease or disorder mediated by complement pathway activation or dysregulation (e.g., activation or dysregulation of C3) using any appropriate nucleic acid delivery method. For example, the oligonucleotides may be delivered to the cell by injecting a solution containing the oligonucleotides, bombardment by particles covered by the oligonucleotides, exposing the cell or organism to a solution containing the oligonucleotides, or electroporation of cell membranes in the presence of the oligonucleotides.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine) can be used. Suitable lipids include Oligofectamine, Lipofectamine (Life Technologies), NC388 (Ribozyme Pharmaceuticals, Inc., Boulder, Colo.), or FuGene 6 (Roche), all of which can be used according to the manufacturer's instructions.

Accordingly, in some embodiments, a formulation comprises a lipid nanoparticle. In some embodiments, an excipient comprises a liposome, a lipid, a lipid complex, a microsphere, a microparticle, a nanosphere or a nanoparticle, or may be otherwise formulated for administration to the cells, tissues, organs, or body of a subject in need thereof (see, e.g., Remington: THE SCIENCE AND PRACTICE OF PHARMACY, 22nd edition, Pharmaceutical Press, 2013).

Effective intracellular concentrations of an oligonucleotide disclosed herein may also be achieved via the stable expression of a polynucleotide encoding the oligonucleotide (e.g., by integration into the nuclear or mitochondrial genome of a mammalian cell) or by the temporary expression in a cell contacted with a polynucleotide (e.g., a plasmid or other vector (e.g., a viral vector) encoding the oligonucleotide). Examples of expression vectors are disclosed in, e.g., WO 1994/011026 and are incorporated herein by reference. Expression vectors for use in the compositions and methods described herein contain an oligonucleotide sequence that reduces C3 expression as well as, e.g., additional sequence elements used for the expression of these agents and/or the integration of these polynucleotide sequences into the genome of a mammalian cell. The expression vector may be a viral vector, a retroviral vector, an adenoviral vector, or an adeno-associated viral vector.

Other methods for delivering oligonucleotides to cells may also be used, such as lipid-mediated carrier transport, chemical-mediated transport, cationic liposome transfection such as calcium phosphate, and vectors including the oligonucleotides. The vectors used for delivery of the oligonucleotides described herein may be viral vectors, such as a retroviral vector (e.g., a lentiviral vector), an adenoviral vector (e.g., Ad5, Ad26, Ad34, Ad35, and Ad48), and an adeno-associated viral vector (AAV) (e.g., AAV1, AAV2, AAV3, AAV4, AAV5, AAV6, AAV7, AAV8, AAV9, and AAV10).

In some examples, an oligonucleotide described herein may be delivered in the form of a transgene that is engineered to express in a cell the oligonucleotides (e.g., its sense and antisense strands). Transgenes may be delivered using a vector, e.g., a viral vector (e.g., adenovirus, retrovirus, vaccinia virus, poxvirus, adeno-associated virus, or herpes simplex virus), as described above or a non-viral vector (e.g., plasmids or synthetic mRNAs). In some embodiments, transgenes can be injected directly into a subject, e.g., at or near the source of action (e.g., within or near the liver) or within the bloodstream.

### C3 Inhibition

Upon administration, the oligonucleotides of the disclosure are capable of binding to and inhibiting the expression of the C3 mRNA. Inhibition of the expression of a C3 gene may be manifested by a reduction of the amount of mRNA expressed by a first cell or group of cells (such cells may be present, for example, in a sample derived (e.g., obtained) from a subject) in which a C3 gene is transcribed and which has or have been treated (e.g., by contacting the cell or cells with an oligonucleotide (e.g., RNAi oligonucleotide) of the disclosure, or by administering an oligonucleotide (e.g., RNAi oligonucleotide) of the disclosure to a subject in which the cells are or were present) such that the expression of a C3 gene is inhibited, as compared to a second cell or group of cells substantially identical to the first cell or group of cells but which has not or have not been so treated (control cell(s) not treated with an oligonucleotide (e.g., RNAi oligonucleotide) or not treated with an oligonucleotide (e.g., RNAi oligonucleotide) targeted to the gene of interest). The level of target mRNA may be measured using techniques well known to one of skill in the art, such as RT-qPCR. The degree of inhibition may be expressed in terms of:

$$\frac{( mRNA in control cells ) - ( mRNA in treated cells )}{( mRNA in control cells )} \times 100 \%$$

A change in the levels of expression of the C3 gene may be assessed in terms of a reduction of a parameter that is functionally linked to C3 gene expression, e.g., C3 protein expression, C3 protein activity, or C3 signaling pathways. C3 gene silencing may be determined in any cell expressing C3, either endogenous or heterologous from an expression construct, and by any assay known in the art.

The consequences of inhibition of the C3 mRNA can be confirmed by an appropriate assay to evaluate one or more properties of a cell or subject, or by biochemical techniques that evaluate molecules indicative of C3 expression (e.g., RNA, protein). The extent to which an oligonucleotide provided herein reduces levels of expression of C3 is evaluated by comparing expression levels to an appropriate control (e.g., a level of C3 mRNA expression in a cell or population of cells to which an oligonucleotide has not been delivered or to which a negative control has been delivered). An appropriate control level of C3 mRNA expression may be a predetermined level or value, such that a control level need not be measured every time. The predetermined level or value can take a variety of forms including a single cut-off value, such as a median or mean. For example, the predetermined level or value may be at or about a level of 75-175 mg/dL of C3 protein, which corresponds to a level of C3 protein that is typically found in the serum of a healthy subject.

The level of expression C3 mRNA in a sample may be determined, for example, by detecting a transcribed polynucleotide, or portion thereof, e.g., mRNA. RNA may be extracted from cells using RNA extraction techniques including, for example, using acid phenol/guanidine isothiocyanate extraction (RNAZOL™ B; Biogenesis), RNEASY™ RNA preparation kits (Qiagen) or PAXGENE™ (PreAnalytix, Switzerland). The C3 mRNA in a sample may also be determined using real-time PCR (RT-PCR). For example, RNA may be extracted by homogenizing tissue samples in QIAzo Lysis reagent using TissueLyser II (Qiagen) and purifying using MAGMAX® Technology (ThermoFisher Scientific) according to the manufacturer's instructions. High-capacity cDNA reverse transcription kits (ThermoFisher Scientific) may then be used to prepare cDNA. Specific primers and probes for C3 and a housekeep control were used for PCR on a CFX384 Real-Time PCR Detection System (Bio-Rad Laboratories), and the BioRad CFX Maestro Software was used to estimate Ct values; the expression level was calculated in EXCEL® and plotted in Prism (GraphPad). The primers used for RT-PCR are described in Table 2.

TABLE 2
Primers used for RT-PCR

| | | | Dye and | SEQ |
|---|---|---|---|---|
| Gene | | | Quencher | ID |
| Target | Primers | Sequence/Probe ID | Label | NO: |
| Mus | mC3 | AAT GAG GCC TTC TCT CTA ACA | N/A | 55 |
| musculus | F4080 | | | |
| (Mm) C3 | | | | |
| Mm C3 | mC3 | ACT TCT TGC AGG TGA CTT TG | N/A | 56 |
| | R4183 | | | |
| Mm C3 | mC3 | /56-FAM/TT GTC GGT G/ZEN/G | FAM/ZEN/IABkFQ | — |
| | P4125 | TGG CAG TGT ATC AT/3IABkFQ/ | | |
| Mm HPRT | Forward | CAA ACT TTG CTT TCC CTG GT | N/A | 57 |
| F576 | | | | |
| Mm HPRT | Reverse | CAA AGT CTG GCC TGT ATC | N/A | 58 |
| R664 | | | | |
| Mm HPRT | Probe | TGGTTAAGGTTGCAAGCTTGCTGGTG | 5′-Hex, Zen, | — |
| P616 | | | 3′-IABkFQ | |
| hC3 F1313 | Forward | CCA AAC TCA GCA TCA ACA CAC | N/A | 59 |
| hC3 R1419 | Reverse | CTG CAT GGT CCT GGT AGC | N/A | 60 |
| hC3 P1339 | Probe | /56-FAM/AG CCA GAA G/ZEN/C | FAM/ZEN/IABKFQ | |
| | | CCT TGA GCA TCA C/3IABkFQ/ | | |
| hC3 F4147 | Forward | GCC AAA GAT CAA CTC ACC TGT | N/A | 61 |
| hC3 R4296 | Reverse | A AGA CAT AGT GGC ATC CTG GT | N/A | 62 |
| hC3 P4197 | Probe | /56-FAM/CC TCT TTT C/ZEN/T | FAM/ZEN/IABKFQ | — |
| | | GTT TCC GGT GCT GGT/3IABkFQ/ | | |
| Monkey C3 | Taqman | Mf02794722_m1 | 5′-FAM | — |
| NeoR-F172 | Forward | AAT GAA CTG CAG GAC GAG G | NA | 63 |
| (Neomycin | Reverse | AGG TGA GAT GAC AGG AGA TCC | NA | 64 |
| resistance) | | | | |
| For HDI | Probe | /5HEX/CAC TGA AGC/ZEN/GGG | 5′-Hex, Zen, | — |
| experiments | | AAG GGA CTG G/3IABkFQ/ | 3′-IABkFQ | |
| RhHPRT1 | RhHPRT1- | CTT TCC TTG GTC AGG CAG TAT | NA | 65 |
| | F583 | | | |
| RhHPRT1 | RhHPRT1- | CAA CAC TTC GTG GAG TCC TT | NA | 66 |
| | R642 | | | |
| RhHPRT1 | RhHPRT1- | /5HEX/CC AAA GAT G/ZEN/G TCA | 5′-HEX, ZEN, | — |
| | R607 | AGG TCG CAA GC/3IABkFQ/ | 3′ IABLFQ | |
| RhPPIB | Taqman | Mf02802985_m1 | 5′-VIC-MGB | — |

Typical assay formats utilizing ribonucleic acid hybridization include nuclear run-on assays, RT-PCR, RNase protection assays, northern blotting, in situ hybridization, and microarray analysis. Circulating mRNA may be detected using methods the described in PCT Publication WO2012/177906, the entire contents of which are hereby incorporated herein by reference. The level of expression of the gene of interest may also be determined using a nucleic acid probe.

Isolated mRNA can be used in hybridization or amplification assays that include, but are not limited to, Northern or southern analyses, polymerase chain reaction (PCR) analyses, and probe arrays. One method for the determination of mRNA levels involves contacting the isolated mRNA with a nucleic acid molecule (probe) that can hybridize to the mRNA of a gene of interest. The mRNA may be immobilized on a solid surface and contacted with a probe, for example by running the isolated mRNA on an agarose gel and transferring the mRNA from the gel to a membrane, such as nitrocellulose. The probe(s) may also be immobilized on a solid surface and the mRNA is contacted with the probe(s), for example, in an AFFYMETRIX® GENECHIP® array. Known mRNA detection methods in the art may be adapted for use in determining the level of mRNA of a gene of interest.

An alternative method for determining the level of expression of a gene of interest in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known in the art. These detection schemes are especially useful for the detection of nucleic acid molecules if such molecules are present in very low numbers. In some aspects of the disclosure, the level of expression of a gene of interest (e.g., C3) is determined by quantitative fluorogenic RT-PCR (i.e., the TAQMANT System) or the DUAL-GLO® Luciferase assay.

The expression levels of mRNA of a gene of interest may be monitored using a membrane blot (such as used in hybridization analysis such as northern, Southern, dot, and the like), or microwells, sample tubes, gels, beads, or fibers (or any solid support including bound nucleic acids). See U.S. Pat. Nos. 5,770,722; 5,874,219; 5,744,305; 5,677,195; and 5,445,934, which are incorporated herein by reference. The determination of gene expression level may also include using nucleic acid probes in solution.

Using the assays described above, a determination can be made about the effectiveness of treatment with the oligonucleotides described herein based on the amount of C3 mRNA reduction. The reduction in levels of C3 mRNA may be a reduction to 1% or lower, 5% or lower, 10% or lower, 15% or lower, 20% or lower, 25% or lower, 30% or lower, 35% or lower, 40% or lower, 45% or lower, 50% or lower, 55% or lower, 60% or lower, 70% or lower, 80% or lower, or 90% or lower compared with an appropriate control level of C3 mRNA or a level of C3 in the subject prior to treatment. The appropriate control level may be a level of C3 mRNA expression in a cell or population of cells that has not been contacted with an oligonucleotide as described herein. In some embodiments, the effect of delivery of an oligonucleotide to a cell according to a method disclosed herein is assessed after a finite period of time. For example, levels of C3 mRNA may be analyzed in a cell at least 8 hours, 12 hours, 18 hours, 24 hours; or at least 3, 4, 5, 10, 15, 20, 30, 40, 50, 60, 70, or 80 days after introduction of the oligonucleotide into the cell.

Furthermore, inhibition the C3 gene may result in the inhibition of C3 protein expression which may be manifested by a reduction in the level of the C3 protein that is expressed by a cell or group of cells (e.g., the level of protein expressed in a sample derived from a subject). As explained above, for the assessment of mRNA suppression, the inhibition of protein expression levels in a treated cell or group of cells may similarly be expressed as a percentage of the level of protein in a control cell or group of cells.

The consequences of inhibition of the C3 protein expression can be confirmed by an appropriate assay to evaluate one or more properties of a cell or subject, or by biochemical techniques that evaluate molecules indicative of C3 protein expression. The extent to which an oligonucleotide provided herein reduces levels of expression of C3 protein is evaluated by comparing expression levels to an appropriate control (e.g., a level of C3 protein expression in a cell or population of cells to which an oligonucleotide has not been delivered or to which a negative control has been delivered). An appropriate control level of C3 protein expression may be a predetermined level or value, such that a control level need not be measured every time, such as an amount of C3 protein determined to be in the normal range, e.g., between 75-175 mg/dL in serum. The predetermined level or value can take a variety of forms including a single cut-off value, such as a median or mean.

The level of C3 protein produced by the expression of the C3 gene may be determined using any method known in the art for the measurement of protein levels. Such methods include, for example, electrophoresis, capillary electrophoresis, high performance liquid chromatography (HPLC), liquid chromatography tandem mass spectrometry (LC/MS/MS), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of proteins produced by the gene of interest. Additionally, the above assays may be used to report a change in the mRNA sequence of interest that results in the recovery or change in protein function thereby providing a therapeutic effect and benefit to the subject, treating a disorder in a subject, and/or reducing of symptoms of a disorder in the subject.

Using the assays described above, a determination can be made about the effectiveness of treatment with the oligonucleotides described herein based on the amount of C3 protein reduction. The reduction in levels of C3 protein may be a reduction to 1% or lower, 5% or lower, 10% or lower, 15% or lower, 20% or lower, 25% or lower, 30% or lower, 35% or lower, 40% or lower, 45% or lower, 50% or lower, 55% or lower, 60% or lower, 70% or lower, 80% or lower, or 90% or lower compared with an appropriate control level of C3 (e.g., about 75-175 mg/dL). The appropriate control level may be a level of C33 expression in a cell or population of cells that has not been contacted with an oligonucleotide as described herein. The effect of delivery of an oligonucleotide to a cell according to a method disclosed herein may be assessed after a finite period of time. For example, levels of C3 may be analyzed in a cell at least 8 hours, 12 hours, 18 hours, 24 hours; or at least one, two, three, four, five, six, seven, or fourteen days after introduction of the oligonucleotide into the cell. The level of C3 may be determined in order to assess whether re-treatment of the subject is needed. For example, if a level of C3 increases to a pre-treatment level (or a level that is at least about 20% or more (e.g., 30%, 40%, 50%, 60%, 70%, 80%, 90%, or more) of the pre-treatment level), the subject may be in need of re-treatment.

Furthermore, inhibition the C3 gene using the methods described herein may result in reducing transcription of C3 mRNA in a cell of a subject identified as having a disease mediated by complement pathway activation or dysregulation. Methods provided herein are useful in any appropriate cell type (e.g., a cell that expresses C3, such as a hepatocyte). In some embodiments, the cell is a primary cell that has been obtained from a subject and that may have undergone a limited number of a passages, such that the cell substantially maintains its natural phenotypic properties. In some embodiments, a cell to which the oligonucleotide is delivered is ex vivo or in vitro (i.e., can be delivered to a cell in culture or to an organism in which the cell resides). In specific embodiments, methods are provided for delivering to a cell an effective amount of an oligonucleotide(s) disclosed herein for purposes of reducing expression of C3 solely in hepatocytes.

An effective amount of an oligonucleotide(s) disclosed herein may be determined as the amount of an oligonucleotide(s) that results in a reduction in symptoms of a disease or disorder mediated by complement pathway activation or dysregulation, such as one of the diseases or disorders described herein. The reduction in symptoms of a disease or disorder mediated by complement pathway activation or dysregulation may be a reduction of at least 10%, at least 20%, at least 30%, at least 40%, at least 50, at least 60%, at least 70%, at least 80%, at least 90%, or 100%, e.g., as determined using clinical assessments known to a person of skill in the art. The amount of reduction in symptoms of a disease or disorder mediated by complement pathway activation or dysregulation may be used to determine if subject is in need of being treated again with an oligonucleotide(s), pharmaceutical composition(s), vector(s), or cell(s) described herein. Examples of assays to determine reduction in a disease mediated by complement pathway activation or dysregulation includes but is not limited to measuring and/or quantifying circulating C3 protein, functional assays (e.g., WEISLAB® assay and hemolytic assay). Quantitation of C3 (or C3 cleavage products) deposition may be performed via IHC or immunofluorescence; and via specific disease biomarkers.

Furthermore, an oligonucleotide described herein that includes both a sense strand and an antisense strand as a duplex polypeptide may be introduced to a cell of a subject using any appropriate nucleic acid delivery. The duplex oligonucleotide may be delivered to the cell by injecting a solution containing the oligonucleotide, bombardment by particles covered by the oligonucleotide, exposing the cell or organism to a solution containing the oligonucleotide, or electroporation of cell membranes in the presence of the oligonucleotide. The duplex oligonucleotides may also be delivered to the cells using lipid-mediated carrier transport, chemical-mediated transport, cationic liposome transfection such as calcium phosphate, and vectors encoding the nucleic acids of the single-strand oligonucleotide. The vectors used for delivery of the duplex oligonucleotide may be viral vectors, such as a retroviral vector (e.g., a lentiviral vector), an adenoviral vector (e.g., Ad5, Ad26, Ad34, Ad35, and Ad48), and an adeno-associated viral vector (AAV) (e.g., AAV1, AAV2, AAV3, AAV4, AAV5, AAV6, AAV7, AAV8, and AAV9).

### Treatment Methods

Also, disclosed herein are methods for the treatment of diseases mediated by complement pathway activation or dysregulation, including, e.g., one or more of the diseases associated with complement pathway activation or dysregulation disclosed herein, in a subject by administration of the compositions described herein (e.g., an oligonucleotide, a vector encoding an oligonucleotide, a cell containing the vector, and a pharmaceutical composition). The method may include the treatment of diseases mediated by complement pathway activation or dysregulation in a subject by administration of a pharmaceutically acceptable salt (e.g., a sodium salt) of the RNAi oligonucleotide described herein. The methods described herein typically involve administering to a subject an effective amount of an oligonucleotide, or pharmaceutically acceptable salt thereof, that is, an amount capable of producing a desirable therapeutic result (e.g., knockdown of C3 expression). A therapeutically acceptable amount may be an amount that is capable of treating a disease or disorder mediated by complement pathway activation or dysregulation (e.g., activation or dysregulation of C3). The appropriate dosage for any one subject will depend on certain factors, including the subject's size, body surface area, age, the particular composition to be administered, the active ingredient(s) in the composition, time and route of administration, general health, and other drugs being administered concurrently. Such treatments could be used, for example, to slow, halt, or prevent any type of disease or disorder mediated by complement pathway activation or dysregulation and may be administered either prophylactically or therapeutically. Administration of a prophylactic agent can occur prior to the detection of, or the manifestation of, symptoms characteristic of the disease or disorder mediated by complement pathway activation or dysregulation, such that the disease or disorder is prevented or, alternatively, delayed in its progression. Subjects at risk for a disease mediated by complement pathway activation or dysregulation can be identified by, for example, one or a combination of diagnostic or prognostic assays known in the art.

The compositions disclosed herein may be administered to a subject using any standard method. For example, any one of the compositions disclosed herein may be administered enterally (e.g., orally, by gastric feeding tube, by duodenal feeding tube, via gastrostomy, or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the liver of a subject). Typically, oligonucleotides disclosed herein are administered intravenously or subcutaneously. The most suitable route for administration in any given case will depend on the particular composition administered, the subject, the particular disease or disorder mediated by complement pathway activation or dysregulation being treated, pharmaceutical formulation methods, administration methods (e.g., administration time and administration route), the subject's age, body weight, sex, severity of the diseases being treated, the subject's diet, and the subject's excretion rate.

The subject suffering from the disease or disorder mediated by complement pathway activation or dysregulation may be administered the oligonucleotides described herein, for example, annually (e.g., once every 12 months), semi-annually (e.g., once every six months), quarterly (e.g., once every three months), bi-monthly (e.g., once every two months), monthly, or weekly. In other instances, the oligonucleotides may be administered every one, two, or three weeks. In certain embodiments, the oligonucleotides may be administered daily.

The subject to be treated for a disease mediated by complement pathway activation or dysregulation may be a human or non-human primate or another mammalian subject (e.g., a human). Other exemplary subjects that may be treated with the oligonucleotides described herein include domesticated animals such as dogs and cats; livestock such as horses, cattle, pigs, sheep, goats, and chickens; and animals such as mice, rats, guinea pigs, and hamsters.

### Dosages

A dosage of the composition of the disclosure (e.g., a composition including an RNAi oligonucleotide, or pharmaceutically acceptable salt thereof, as described herein) can vary depending on many factors, such as the pharmacodynamic properties of the compound, the mode of administration, the age, health, and weight of the recipient, the nature and extent of the symptoms, the frequency of the treatment and/or the type of concurrent treatment, if any, and the clearance rate of the compound in the subject to be treated. One of skill in the art can determine the appropriate dosage based on the above factors.

The oligonucleotides of the disclosure, or pharmaceutically acceptable salts thereof, may be administered in an amount and for a time effective to result in one or more of (e.g., 2 or more, 3 or more, 4 or more of): (a) decreased expression of C3 protein in a cell of the subject, (b) reduced transcription of C3 in the cell of the subject, (c) reduced level of C3 protein in the cell of the subject, (d) reduced activity of the C3 protein the in cell of the subject; and/or (e) reduction in one or more symptoms of a disease or disorder mediated by complement pathway activation or dysregulation.

Accordingly, the disclosure relates to a method for treating a disease mediated by complement pathway activation or dysregulation in a subject in need thereof, in which the method includes administering an effective amount of an oligonucleotide described that binds specifically to C3 mRNA and inhibits expression of C3 protein in the subject. For example, the disclosure provides a method of treating a disease mediated by complement pathway activation or dysregulation in a subject in need thereof including administering to the subject a therapeutically effective amount of an oligonucleotide, pharmaceutical composition, vector, or cell disclosed herein.

The disease mediated by complement pathway activation or dysregulation to be treated utilizing the disclosed methods and compositions may be, e.g., one or more of the diseases associated with complement pathway activation or dysregulation disclosed herein.

The treatment of diseases mediated by complement pathway activation or dysregulation can be accomplished by administration of an oligonucleotide (e.g., an RNAi oligonucleotide) that inhibits the expression and/or translation of C3 mRNA (e.g., the expression of C3 protein), such as those described herein.

The disclosed compositions can be administered in amounts determined to be appropriate by those of skill in the art. In some embodiments, the oligonucleotide described herein may be administered initially in a suitable dosage that may be adjusted as required, depending on the clinical response. In some instances, the oligonucleotide, or pharmaceutically acceptable salt thereof, is administered at a dose of 0.01-100 mg/kg (e.g., 0.01-1 mg/kg, 1-5 mg/kg, 5-20 mg/kg, 20-50 mg/kg, 50-100 mg/kg) of bodyweight of a subject. In certain instances, the oligonucleotide is administered at a concentration of 0.01 mg/kg-50 mg/kg (e.g., 0.01-1 mg/kg, 1-5 mg/kg, 5-10 mg/kg, 10-20 mg/kg, 20-30 mg/kg, 30-40 mg/kg, 40-50 mg/kg) bodyweight of the subject. In other instances, the oligonucleotide is administered at a concentration of 0.01 mg/kg-20 mg/kg (e.g., 0.01-1 mg/kg, 1-5 mg/kg, 5-10 mg/kg, 10-15 mg/kg, 15-20 mg/kg) bodyweight of the subject. In other instances, the oligonucleotide is administered at a concentration of 0.01 mg/kg-15 mg/kg (e.g., 0.01-1 mg/kg, 1-2 mg/kg, 2-5 mg/kg, 5-8 mg/kg, 8-10 mg/kg, 10-12 mg/kg, 12-15 mg/kg) bodyweight of the subject. In other instances, the oligonucleotide is administered at a concentration of 0.01 mg/kg-10 mg/kg (e.g., 0.01-1 mf/kg, 1-2 mg/kg, 2-5 mg/kg, 5-8 mg/kg, 8-10 mg/kg) bodyweight of the subject. In other instances, the oligonucleotide is administered at a concentration of 0.01 mg/kg-5 mg/kg (e.g., 0.01-1 mg/kg, 1-2 mg/kg, 2-3 mg/kg, 3-4 mg/kg, 4-5 mg/kg) bodyweight of the subject. In other instances, the oligonucleotide is administered at a concentration of 0.1 mg/kg-20 mg/kg (0.1-1 mg/kg, 1-5 mg/kg, 5-10 mg/kg, 10-15 mg/kg, and 15-20 mg/kg) bodyweight of the subject. In other instances, the oligonucleotide is administered at a concentration of 0.1 mg/kg-10 mg/kg (e.g., 0.1-1 mg/kg, 1-2 mg/kg, 2-5 mg/kg, 5-7 mg/kg, and 7-10 mg/kg) bodyweight of the subject. In other instances, the oligonucleotide is administered at a concentration of 0.1 mg/kg-5 mg/kg (e.g., 0.1-1 mg/kg, 2-3 mg/kg, 3-4 mg/kg, and 4-5 mg/kg) bodyweight of the subject. In other instances, the oligonucleotide is administered at a concentration of 1 mg/kg-50 mg/kg (e.g., 1-10 mg/kg, 10-20 mg/kg, 20-30 mg/kg, 30-40 mg/kg, and 40-50 mg/kg) bodyweight of the subject. In other instances, the oligonucleotide is administered at a concentration of 1 mg/kg-20 mg/kg (e.g., 1-5 mg/kg, 5-10 mg/kg, 10-15 mg/kg and 15-20 mg/kg) bodyweight of the subject. In other instances, the oligonucleotide is administered at a concentration of 1 mg/kg-10 mg/kg (e.g., 1-2 mg/kg, 2-5 mg/kg, 5-7 mg/kg, and 7-10 mg/kg) bodyweight of the subject. In other instances, the oligonucleotide is administered at a concentration of 1 mg/kg-5 mg/kg (e.g., 1-2 mg/kg, 2-3 mg/kg, 3-4 mg/kg, and 4-5 mg/kg) bodyweight of the subject. In other instances, the oligonucleotide is administered at a concentration of 30 mg/kg-300 mg/kg (e.g., 30-200 mg/kg, 30-100 mg/kg, 30-50 mg/kg, 50-300 mg/kg, 100-300 mg/kg, 200-300 mg/kg, and 250-300 mg/kg).

In certain embodiments, the oligonucleotide is administered at a dose of less than 10 mg/kg (e.g., 9 mg/kg or less, 8 mg/kg or less, 7 mg/kg or less, 6 mg/kg or less, 5 mg/kg or less, 4 mg/kg or less, 3 mg/kg or less, 2 mg/kg or less, 1 mg/kg or less) bodyweight of the subject. In other embodiments, the oligonucleotide is administered at a dose of about 10 mg/kg or less. In another embodiment, the oligonucleotide is administered at a dose of about 9 mg/kg or less (e.g., 8.9 mg/kg, 8 mg/kg, 7 mg/kg, 5 mg/kg, 3 mg/kg, and 1 mg/kg or less). In other embodiments, the oligonucleotide is administered at a dose of about 8 mg/kg or less (e.g., 7.9 mg/kg, 7 mg/kg, 5 mg/kg, 3 mg/kg, and 1 mg/kg or less). In another embodiment, the oligonucleotide is administered at a dose of about 7 mg/kg or less (e.g., 6.9 mg/kg, 6 mg/kg, 4 mg/kg, 2 mg/kg, and 1 mg/kg or less). In another embodiment, the oligonucleotide (e.g., RNAi oligonucleotide) is administered at a dose of about 6 mg/kg or less (e.g., 5.9 mg/kg, 5 mg/kg, 3 mg/kg, and 1 mg/kg or less). In another embodiment, the oligonucleotide is administered at a dose of about 5 mg/kg or less (e.g., 4.9 mg/kg, 4 mg/kg, 3 mg/kg, 2 mg/kg, and 1 mg/kg or less). In another embodiment, the oligonucleotide is administered at a dose of about 4 mg/kg or less (e.g., 3.9 mg/kg, 3 mg/kg, 2 mg/kg, and 1 mg/kg or less). In another embodiment, the oligonucleotide is administered at a dose of about 3 mg/kg or less (e.g., 2.9 mg/kg, 2.5 mg/kg, 2 mg/kg, 1 mg/kg or less). In another embodiment, the oligonucleotide is administered at a dose of about 2 mg/kg or less (e.g., 1.9 mg/kg, 1.5 mg/kg, 1 mg/kg, and 0.5 mg/kg or less). In another embodiment, the oligonucleotide is administered at a dose of about 1 mg/kg or less (e.g., 0.9 mg/kg, 0.8 mg/kg, 0.7 mg/kg, 0.6 mg/kg, 0.5 mg/kg, 0.4 mg/kg, 0.3 mg/kg, 0.2 mg/kg, and 0.1 mg/kg or less).

In another embodiment, the oligonucleotide is administered at a dose of about 0.1-10 mg/kg, about 0.2-10 mg/kg, about 0.3-10 mg/kg, about 0.4-10 mg/kg, about 0.5-10 mg/kg, about 1-10 mg/kg, about 2-10 mg/kg, about 3-10 mg/kg, about 4-10 mg/kg, about 5-10 mg/kg, about 6-10 mg/kg, about 7-10 mg/kg, about 8-10 mg/kg, or about 9 mg/kg of bodyweight of a subject.

In other instances, the dosage of a composition (e.g., a composition including an RNAi oligonucleotide described herein) is a prophylactically or a therapeutically effective amount. In some cases, a viral vector (e.g., an rAAV vector) is administered at a dose of 10⁵, 10⁶, 10⁷, 10⁸, 10⁹, 10¹⁰, 10¹¹, 10¹², 10¹³, 10¹⁴, or 10¹⁵ genome copies (GC) per subject. In some embodiments the rAAV is administered at a dose of 10⁵, 10⁶, 10⁷, 10⁸, 10⁹, 10¹⁰, 10¹¹, 10¹², 10¹³, or 10¹⁴ GC/kg (total weight of the subject).

Optionally, the disclosed oligonucleotides may be administered as part of a pharmaceutically acceptable composition suitable for delivery to a subject, as is described herein. The disclosed agents are included within these compositions in amounts sufficient to provide a desired dosage and/or elicit a therapeutically beneficial effect, as can be readily determined by those of skill in the art.

The disclosed compositions described herein may be administered in an amount (e.g., an effective amount) and for a time sufficient to treat the subject or to effect one of the outcomes described above (e.g., a reduction in one or more symptoms of disease in the subject). The disclosed compositions may be administered once or more than once. The disclosed compositions may be administered once daily, twice daily, three times daily, once every two days, once weekly, twice weekly, three times weekly, once biweekly, once monthly, once bimonthly, twice a year, or once yearly. Treatment may be discrete (e.g., an injection) or continuous (e.g., treatment via an implant or infusion pump). Subjects may be evaluated for treatment efficacy 1 week, 2 weeks, 1 month, 2 months, 3 months, 4 months, 5 months, 6 months, or more following administration of a composition of the disclosure depending on the composition and the route of administration used for treatment. Subjects may be treated for a discrete period of time (e.g., 1, 2, 3, 4, 5, 6, 7, 8, 9, 10, 11, or 12 months) or until the disease or condition is alleviated, or treatment may be chronic depending on the severity and nature of the disease or condition being treated (e.g., for the life of the subject). For example, a subject diagnosed with PNH and treated with a composition disclosed herein may be given one or more (e.g., 1, 2, 3, 4, 5, 6, 7, 8, 9, 10, or more) additional treatments if initial or subsequent rounds of treatment do not elicit a therapeutic benefit including reduction of any one of the symptoms associated with PNH, such as fatigue, weakness, shortness of breath, bruising or bleeding easily, recurring infections, severe headache, blood clots, and difficulty controlling bleeding, or a reduction in the levels of C3 mRNA or C3 protein levels in the cells or serum of the subject.

### Kits

The disclosure also features kits including (a) a pharmaceutical composition including an oligonucleotide (e.g., an RNAi oligonucleotide) agent, or pharmaceutically acceptable salt thereof, that reduces the level and/or activity of C3 in a cell or subject described herein and, optionally, a pharmaceutically acceptable carrier, excipient, or diluent. The kit may contain a vector encoding an oligonucleotide(s) (e.g., an RNAi oligonucleotide(s)) described herein or a cell including a vector encoding an oligonucleotide(s) (e.g., an RNAi oligonucleotide(s)) described herein. The kit may also include a package insert with instructions to perform any of the methods described herein. In some embodiments, the kit includes (a) a pharmaceutical composition including an oligonucleotide (e.g., RNAi oligonucleotide) agent that reduces the level and/or activity of C3 in a cell or subject described herein, (b) an additional therapeutic agent, and (c) a package insert with instructions to perform any of the methods described herein.

### EXAMPLES

The following examples are intended as illustration only, are not meant to limit the disclosure in any way.

### Example 1: Preparation of RNAi Oligonucleotides

### Oligonucleotide Synthesis and Purification

The RNAi oligonucleotides described in this and the foregoing Examples were chemically synthesized using methods described herein. Generally, RNAi oligonucleotides were synthesized using solid phase oligonucleotide synthesis methods as described for 19-23mer siRNAs (see, e.g., Scaringe et al. (1990) Nucleic Acids Res. 18:5433-5441 and Usman et al. (1987) J. Am. Chem. Soc. 109:7845-7845; see also, U.S. Pat. Nos. 5,804,683; 5,831,071; 5,998,203; 6,008,400; 6,111,086; 6,117,657; 6,353,098; 6,362,323; 6,437,117 and 6,469,158) in addition to using known phosphoramidite synthesis (see, e.g. Hughes and Ellington (2017) COLD SPRING HARB PERSPECT BIOL. 9 (1): a023812; Beaucage S. L., Caruthers M. H., Studies on Nucleotide Chemistry V: Deoxynucleoside Phosphoramidites—A New Class of Key Intermediates for Deoxypolynucleotide Synthesis, TETRAHEDRON LETT. 1981; 22:1859-1862. doi: 10.1016/S0040-4039 (01) 90461-7).

RNAi oligonucleotides having a 19mer core sequence were formatted into constructs having a 25mer sense strand and a 27mer antisense strand to allow for processing by the RNAi machinery. The 19mer core sequence was complementary to a region in the C3 mRNA.

Individual RNA strands were synthesized and HPLC purified according to standard methods (Integrated DNA Technologies; Coralville, IA). For example, RNA oligonucleotides were synthesized using solid phase phosphoramidite chemistry, deprotected and desalted on NAP-5 columns (Amersham Pharmacia Biotech; Piscataway, NJ) using standard techniques (Damha & Olgivie (1993) METHODS MOL. BIOL. 20:81-114; Wincott et al. (1995) NUCLEIC ACIDS RES. 23:2677-2684). The oligomers were purified using ion-exchange high performance liquid chromatography (IE-HPLC) on an Amersham Source 15Q column (1.0 cm×25 cm; Amersham Pharmacia Biotech) using a 15 min step-linear gradient. The gradient varied from 90:10 Buffers A:B to 52:48 Buffers A:B, where Buffer A is 100 mM Tris pH 8.5 and Buffer B is 100 mM Tris pH 8.5, 1 M NaCl. Samples were monitored at 260 nm and peaks corresponding to the full-length oligonucleotide species were collected, pooled, desalted on NAP-5 columns, and lyophilized.

The purity of each oligomer was determined by capillary electrophoresis (CE) on a Beckman PACE 5000 (Beckman Coulter, Inc.; Fullerton, CA). The CE capillaries have a 100 μm inner diameter and contain ssDNA 100R Gel (Beckman-Coulter). Typically, about 0.6 nmole of oligonucleotide was injected into a capillary, run in an electric field of 444 V/cm and was detected by UV absorbance at 260 nm. Denaturing Tris-Borate-7 M-urea running buffer was purchased from Beckman-Coulter. Oligoribonucleotides were obtained that were at least 90% pure as assessed by CE for use in experiments described below. Compound identity was verified by matrix-assisted laser desorption ionization time-of-flight (MALDI-TOF) mass spectroscopy on a VOYAGER-DET BIOSPECTROMETRY™ Work Station (Applied Biosystems; Foster City, CA) following the manufacturer's recommended protocol. Relative molecular masses of all oligomers were obtained, often within 0.2% of expected molecular mass.

### Preparation of Duplexes

Single strand RNA oligomers were resuspended (e.g., at 100 UM concentration) in duplex buffer consisting of 100 mM potassium acetate, 30 mM HEPES, pH 7.5. Complementary sense and antisense strands were mixed in equal molar amounts to yield a final solution of, for example, 50 UM duplex. Samples were heated to 100° C. for 5′ in RNA buffer (IDT) and were allowed to cool to room temperature before use. The RNAi oligonucleotides were stored at −20° C. Single strand RNA oligomers were stored lyophilized or in nuclease-free water at −80° C.

### Example 2: Generation of C3-Targeting RNAi Oligonucleotides

Identification of C3 mRNA Target Sequences

Complement is a tightly regulated enzymatic cascade that can be activated by several different pathways, including the complement classical pathway (CCP), in which antibody complexes trigger activation. Regardless of which pathway starts the process, complement activation converges at C3 in the cascade. Once activated, C3 is cleaved to form the effector molecules C3a and C3b, leading to inflammation, deposition of C3b in tissues, and terminal complement activation and further tissue damage.

To generate RNAi oligonucleotide inhibitors of C3 expression, a computer-based algorithm was used to computationally identify C3 mRNA target sequences suitable for assaying inhibition of C3 expression by the RNAi pathway. Over 300 RNAi oligonucleotide guide (antisense) strand sequences, each having a region of complementarity to a suitable C3 target sequence of human C3 mRNA (see Table 3), were prepared and assayed in vitro for C3 expression inhibition. From these RNAi oligonucleotides, a subset of nine (see Table 4) were selected for further study. The subset of nine guide sequences identified by the algorithm were also complementary to the corresponding C3 target sequence of monkey C3 mRNA (SEQ ID NO: 67; Table 3). C3 RNAi oligonucleotides comprising a region of complementarity to homologous C3 mRNA target sequences with nucleotide sequence similarity are predicted to have the ability to target homologous C3 mRNAs.

TABLE 3
Sequences of Human and Monkey C3 mRNA

| Species | Ref Seq # | SEQ ID NO |
|---|---|---|
| Human (Hs) | NM_000064.4 | 12 |
| Cynomolgus monkey (Mf) | XM_005587719.2 | 67 |

### Example 3: Identification of RNAi Oligonucleotides to Inhibit C3 Expression In Vitro

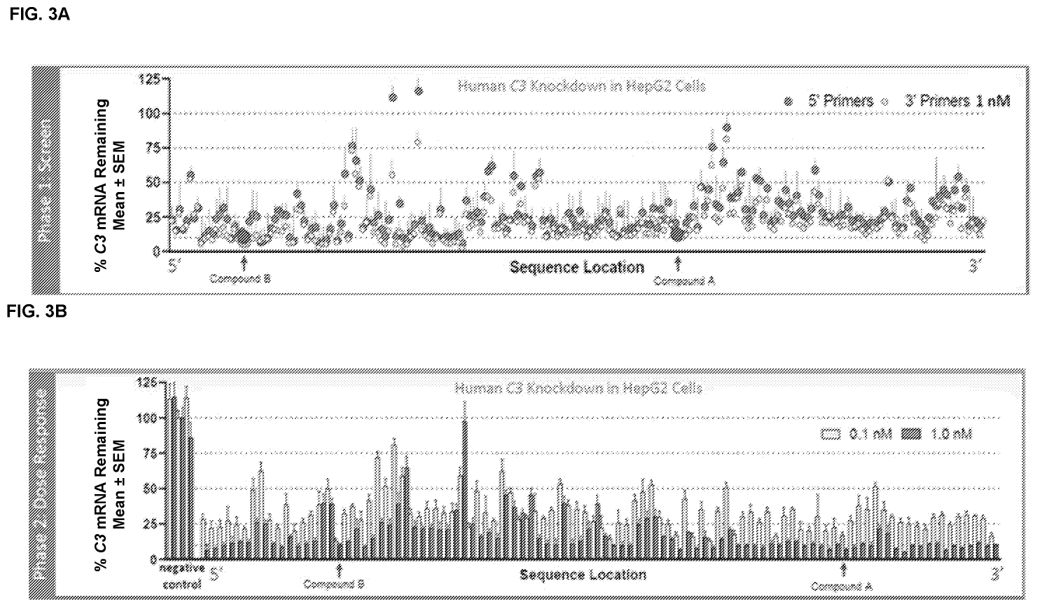

The activity of RNAi oligonucleotides (formatted as dsiRNA oligonucleotides) generated as described in Examples 1 and 2 to reduce C3 mRNA was measured using in vitro cell-based assays. Briefly, HepG2 human liver cells expressing endogenous C3 were transfected with the RNAi oligonucleotides at 1 nM (FIG. 3A) or with a subset of the RNAi oligonucleotide screened in FIG. 3A at two different concentrations (0.1 and 1 nM) (FIG. 3B) as indicated in separate wells of a multi-well cell-culture plate. Cells were maintained for 24 hr following transfection, and then levels of C3 mRNA from the transfected cells were determined using TAQMAN®-based qPCR assays. Two RT-qPCR assays, a 3′ assay and a 5′ assay, were used to determine mRNA levels as measured by HEX and FAM probes, respectively. A subset of RNAi oligonucleotide candidates were selected for further in vivo analysis based on the inhibition of C3 mRNA levels as determined by RT-qPCR.

### Example 4: Screening of RNAi Oligonucleotides in Mice Expressing Human C3 cDNA (HDI Mice)

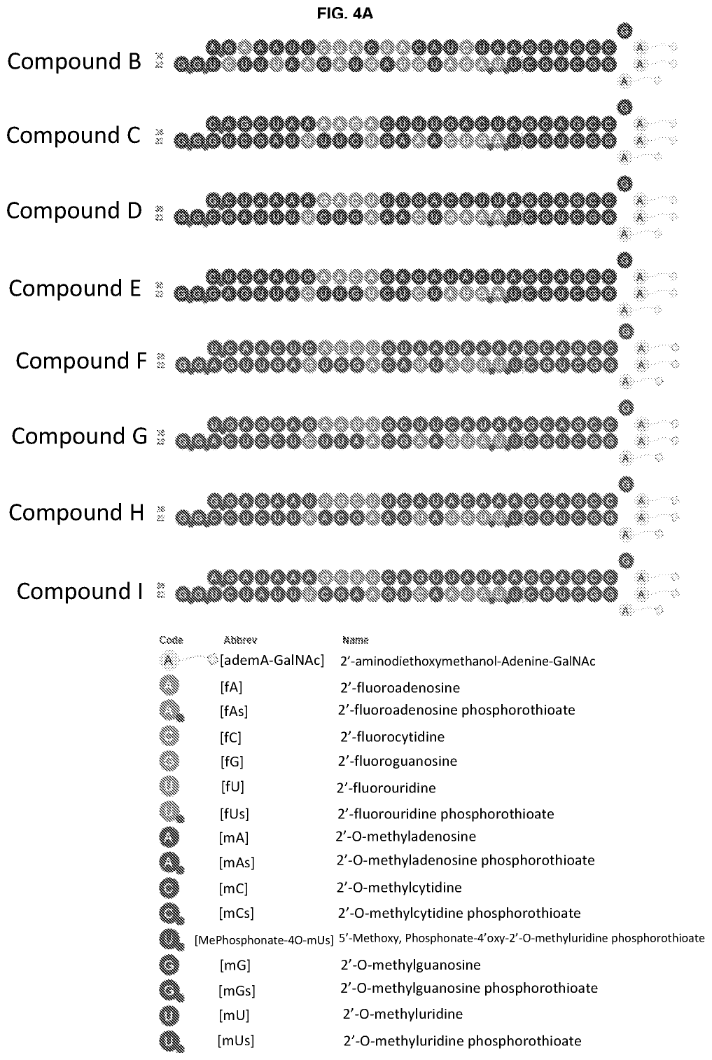

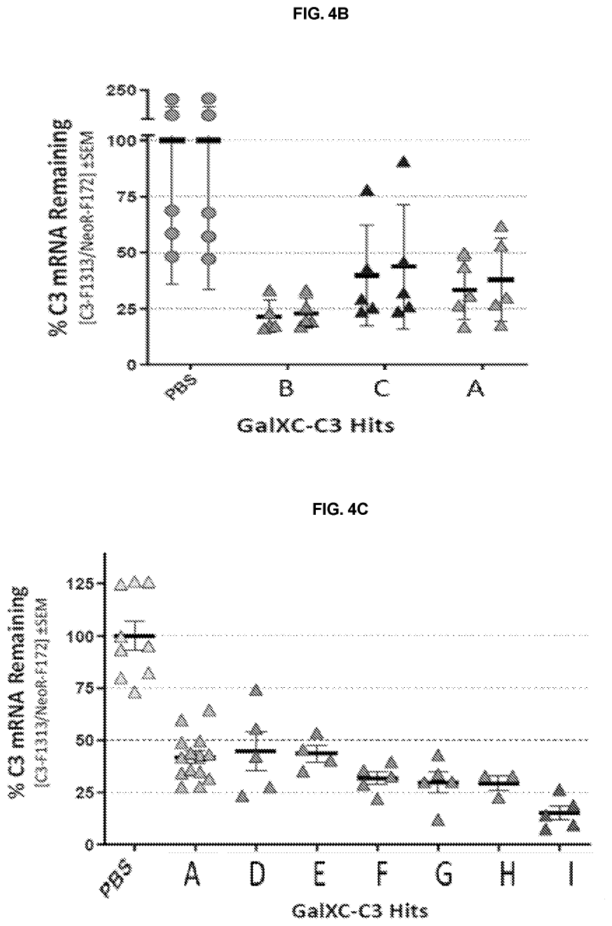

The subset of RNAi oligonucleotide candidates (or “Compounds”) from Example 3 were screened in mice expressing the human C3 cDNA. CD-1 mice transfected with vectors expressing the human C3 cDNA were administered a single subcutaneous dose of selected Compounds (Compounds A-I) at 0.5 or 1 mg/kg. Animals were sacrificed after 4 days for evaluation of human C3 mRNA levels from liver homogenates as determined by RT-qPCR using specific probes. Compounds that showed at least 50% knockdown potency in transfected mice were selected for testing in cynomolgus macaques. The results of the in vivo screening of a subset of 9 Compounds (i.e., Compounds A, B, C, D, E, F, G, H, and I) are depicted in FIGS. 4B and 4C and their corresponding sense and antisense strands are summarized in Table 4 and in FIG. 4A. Data are expressed as percentage of C3 mRNA remaining in the liver relative to PBS treated mice.

TABLE 4
Summary of sense and antisense strands of Compounds A-I

| | Sense | Sense Strand | Antisense | Antisense |
|---|---|---|---|---|
| | Strand | with | Strand | Strand with |
| | SEQ | Modifications | SEQ ID | Modifications |
| Compound | ID NO: | SEQ ID NO: | NO: | SEQ ID NO: |
| A | 1 | 37 | 3 | 38 |
| B | 4 | 39 | 6 | 40 |
| C | 17 | 41 | 18 | 42 |
| D | 18 | 43 | 20 | 44 |
| E | 21 | 45 | 22 | 46 |
| F | 23 | 47 | 24 | 48 |
| G | 25 | 49 | 26 | 50 |
| H | 27 | 51 | 28 | 52 |
| I | 29 | 53 | 30 | 54 |

### Example 5: Screening of RNAi Oligonucleotides in Cynomolgus Macaques

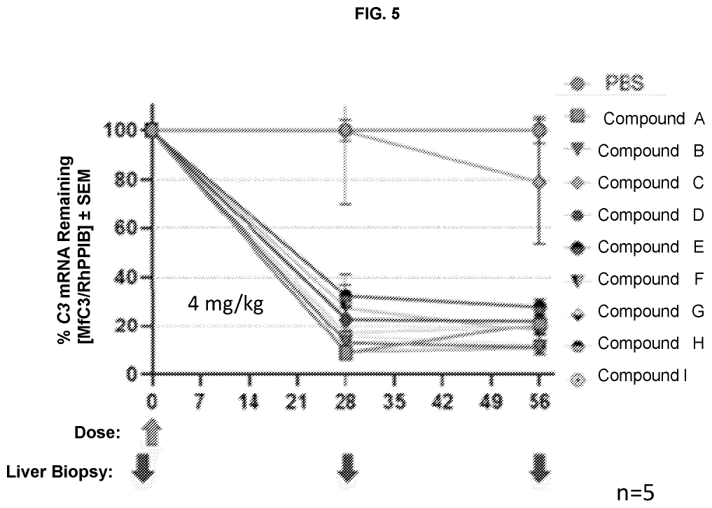

All Compounds A to I, as described in Example 4, Table 4 and FIG. 4A, pre-selected during the mouse screening were tested in cynomolgus macaques (NHP) for duration of C3 mRNA silencing after a single subcutaneous administration of the Compounds A-I at 4 mg/kg. Liver biopsy of all tested animals (n=5/Compound) were collected before dosing and on Day 28 and Day 56 post injection. As demonstrated on FIG. 5, there was at least 50% reduction of liver C3 mRNA levels for most Compounds tested in comparison to normalized baseline levels and time-matched PBS controls as determined by RT-qPCR. Two lead Compounds (Compounds A and B) were selected based on the knockdown level of C3 mRNA in the liver of cynomolgus macaques post single administration for testing in a multidose study.

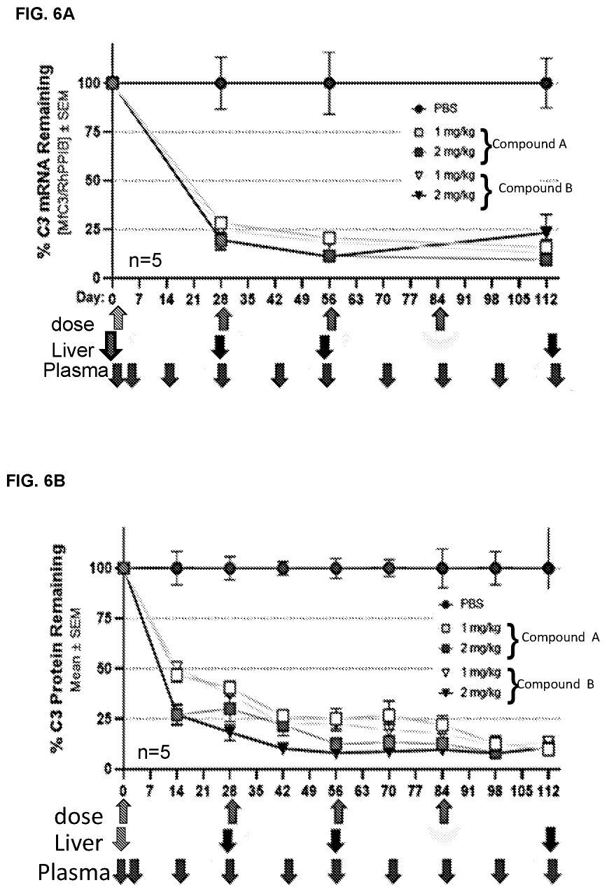

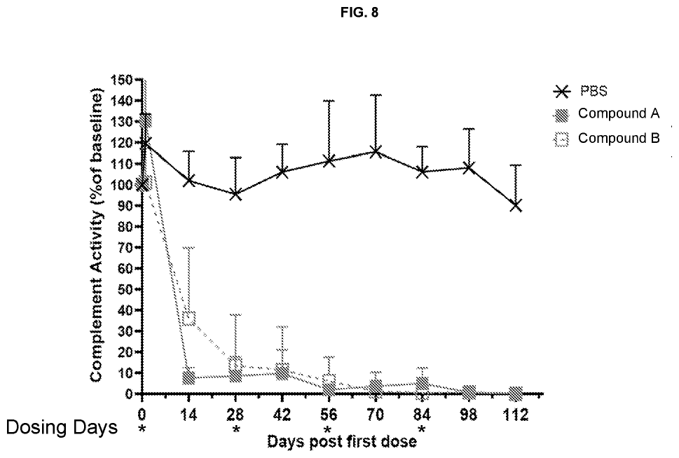

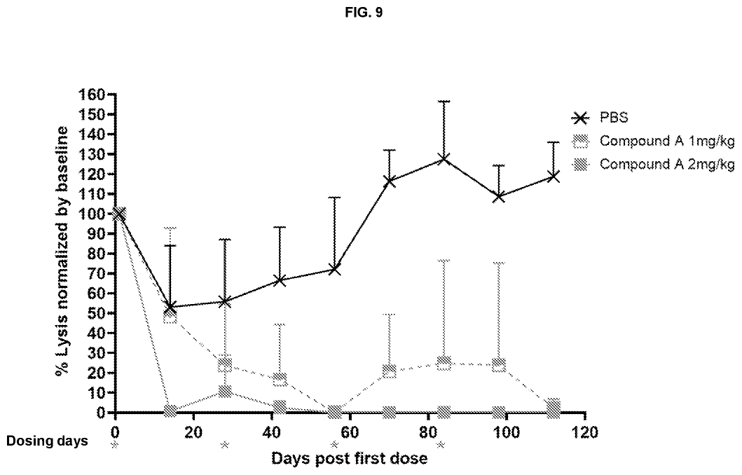

Compounds A and B were selected from the single dose study for further evaluation in a multiple dose NHP study. Cynomolgus macaques were dosed subcutaneously with 1 mg/kg or 2 mg/kg on day 0, day 28, day 56, and day 84 for a total of 4 doses. Liver biopsies were collected pre-dosing and on Days 28, 56 and 112 post initial treatment for evaluation of liver C3 mRNA levels by RT-qPCR (FIG. 6A). Serum samples were collected on pre-dosing, Day 1, 14. 28, 42, 56, 70, 84, 98 and 112 post initial dose for evaluation of C3 protein levels by C3 ELISA kit (FIG. 6B), complement activity by WIESLAB® AP assay (FIG. 8) and by hemolysis of rabbit erythrocytes (FIG. 9). PBS-treated animals were used as control from C3 liver mRNA, C3 serum protein and functional assays. Multiple treatment of cynomolgus macaques with Compound A or B led to a sustained duration of C3 mRNA silencing in the liver, significant reduction of circulating C3 in serum, a >95% reduction of alternative pathway complement activity, and complete inhibition of lysis of rabbit erythrocytes in a hemolytic assay after multiple administrations of Compounds A and B, as depicted in FIGS. 6A, 6B, 8, and 9, respectively.

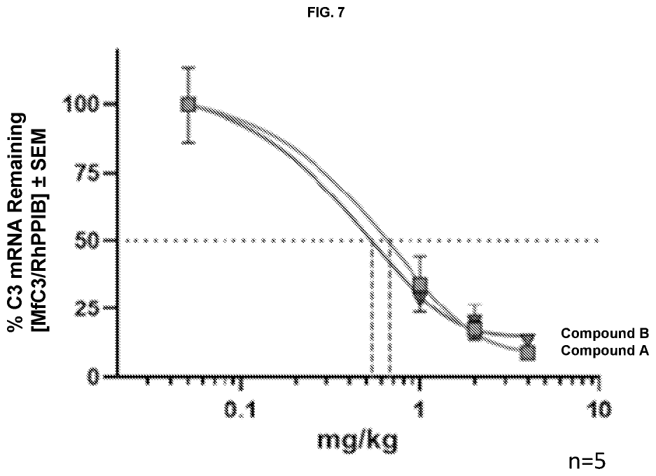

The potency of Compounds A and B was calculated by combining Day 28 results for both single and multidose NHP studies. The approximate ED₅₀ for Compounds A (0.65 mg/kg) and for Compound B (0.55 mg/Kg) was calculated from a dose-response curve generated for both Compounds (FIG. 7).

### Example 6: Pharmacokinetic and Pharmacodynamic Study of Compound J on C3 Expression in CD-1 Mice

CD-1 mice were treated with Compound J (a murine surrogate for Compound A) to assess the percent of liver C3 mRNA knockdown and the serum C3 protein levels in mice as a result of Compound J administration. The percent knockdown of liver C3 mRNA, as a result of Compound J administration, was measured using RT-qPCR. The amount of C3 in serum was measured using a mouse C3 ELISA assay. Mice received a single, subcutaneous dose of Compound J at 0.5 mg/kg, 1 mg/kg, or 6 mg/kg. The single administration of Compound J showed a dose-dependent liver C3 mRNA knockdown percentage of liver C3 mRNA, with greater than 90% reduction of C3 mRNA in the liver from animals that received 6 mg/kg dose (n=5 mice/timepoint). The nadir of mRNA knockdown was 3-14 days after 6 mg/Kg dose as shown in FIG. 10A. The percentage of C3 protein in the serum of CD-1 mice was measured over the course of the study and was correspondingly suppressed (FIG. 10B).

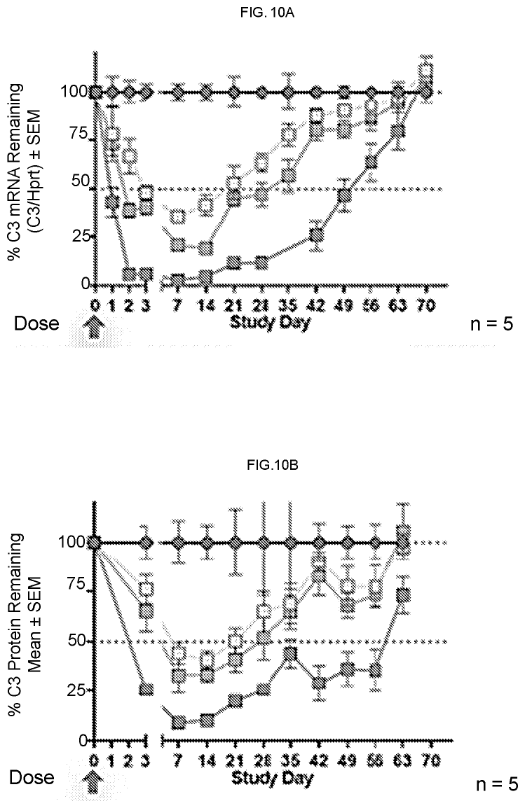

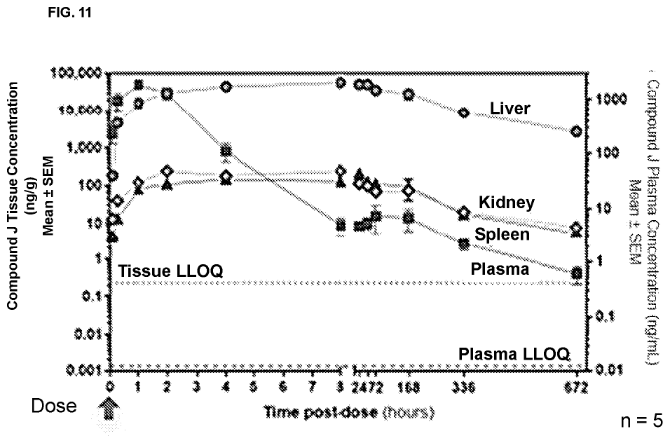

The amount of Compound J in plasma, liver, kidney, and spleen tissues of CD-1 mice which were administered a single, subcutaneous dose of 6 mg/kg of Compound J was measured using stem loop-qPCR over a period of 672 hours after receiving the dose (FIG. 11). Pharmacokinetic analysis indicated that the highest exposure of Compound J was in liver, followed by spleen, kidney, and plasma (FIG. 11).

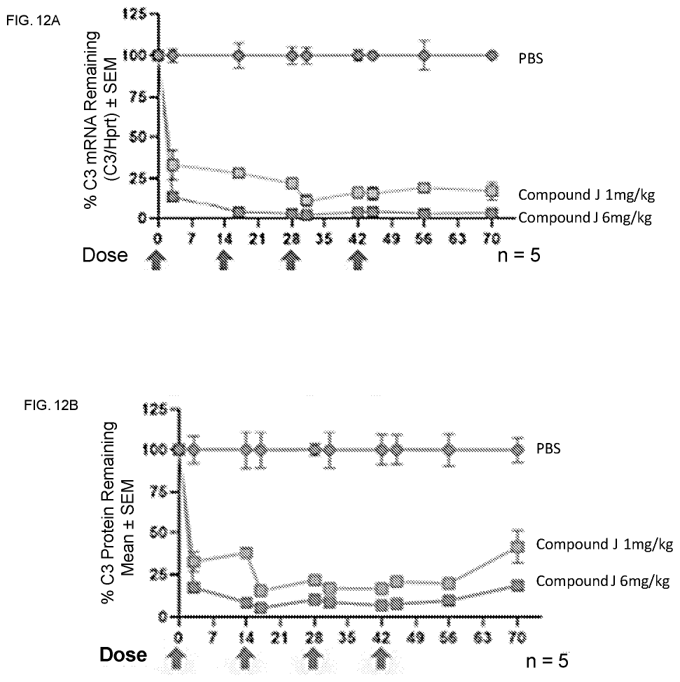

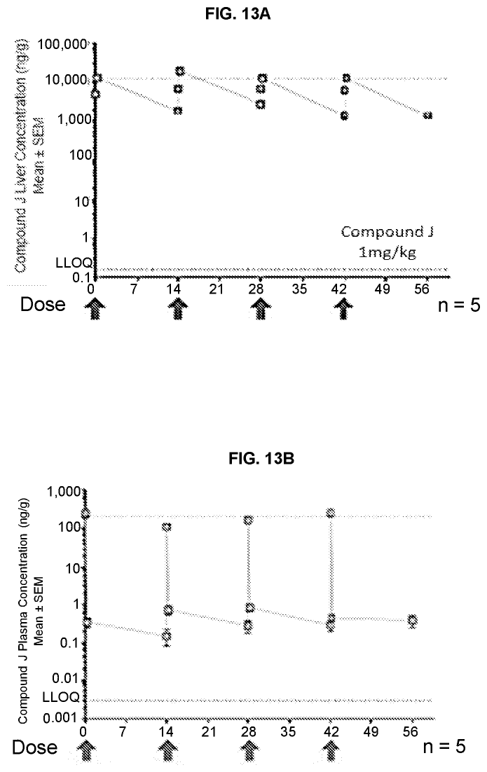

In a multidose study conducted over a 70-day period, the percent C3 mRNA was measured using RT-qPCR, and the amount of C3 protein in serum was measured by mouse C3 ELISA assay, where CD-1 mice received four doses of 1 mg/kg or 6 mg/kg of Compound J on days 0, 14, 28, and 42 as shown in FIGS. 12A and 12B, respectively. This regimen resulted in ˜75% and >95% knockdown of hepatic C3 mRNA and serum protein levels, respectively. Liver biopsies and serum collections were performed on day 3, 14, 17, 28, 31, 42, 45, 56, and 70 after the initial dose. The liver and plasma concentration of Compound J after 4 doses of 1 mg/kg was analyzed from the liver biopsies and plasma samples using Stem Loop qPCR (SL-qPCR) as shown in FIGS. 13A and 13B, respectively. PBS-treated CD-1 mice were used as control for both C3 liver mRNA and C3 serum protein levels.

This multidose study showed that Compound J (a murine surrogate for Compound A) showed a dose-dependent knockdown of liver C3 mRNA that was sustained over the course of 70 days. The reduction of circulating C3 protein levels corresponded to the reduction of C3 mRNA observed in the liver. Additionally, plasma and liver concentrations of Compound J from dosed animals showed no accumulation of Compound J with biweekly dosing (1 mg/kg) (FIGS. 13A and 13B, respectively).

### Example 7: Effect of Compound J on C3 Expression in NZB/W F1 Mouse, a Lupus Nephritis Model

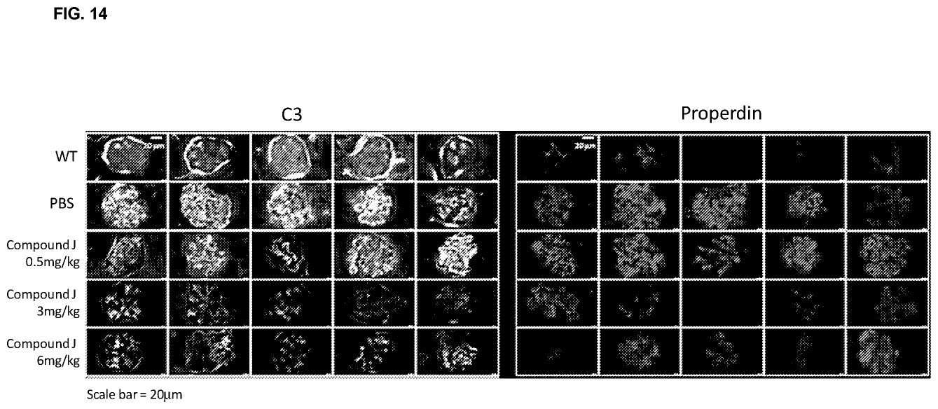

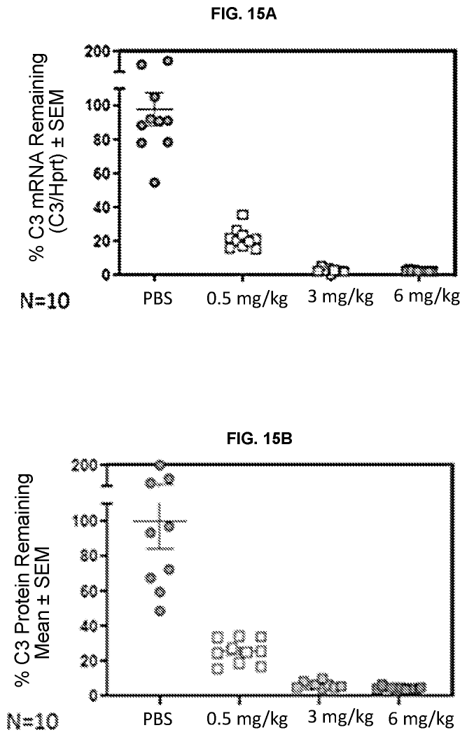

An NZB/W F1 lupus mouse model was used to test proof-of-mechanism in a disease model with Compound J. NZB/W F1 animals were subcutaneously administered with 0.5 mg/kg, 3 mg/kg, or 6 mg/kg doses of Compound J every 4 weeks beginning at the age of 21 weeks (n=10/group). PBS-treated animals were used as negative control and kidneys from CD1-mice were used as non disease control. At the age of 37 weeks, C3 and properdin glomerular deposition was assessed by immunoflurescence imaging of kidneys from Compound J-treated and PBS-control animals (FIG. 14). At, 29 weeks of age, the percent of liver C3 mRNA was measured using RT-qPCR (FIG. 15A), and the amount of C3 protein in serum was quantified using a mouse C3 ELISA assay (FIG. 15B) for each dose level of Compound J. After multiple doses of treatment with Compound J, there was a dose dependent reduction of C3 and properdin glomerular deposition observed from Compound J-treated animals. Multidose treament of NZB/W F1 mice with Compound J showed a dose-dependent knockdown of liver C3 mRNA that was sustained over the course of 16 weeks. The reduction of circulating C3 protein levels (FIG. 15A) corresponded to the reduction of C3 mRNA observed in the liver (FIG. 15B).

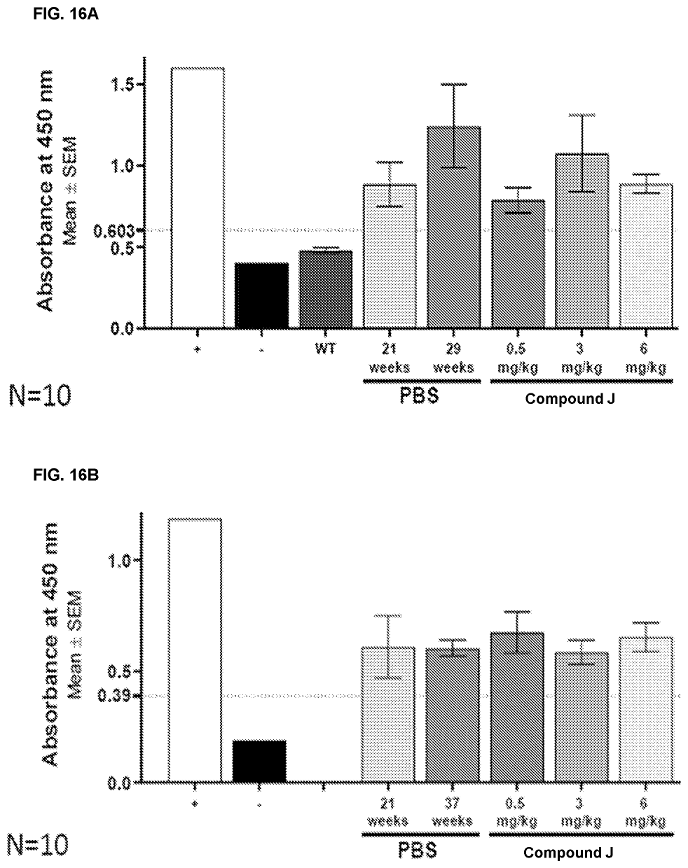

Serum samples were collected at 29 and 37 weeks of age, after 8 and 16 weeks of treatment with Compound J, respectively, to measure circulating IgG immune complexes (CIC) by ELISA assay (FIG. 16A and FIG. 16B). After multiple doses of treatment with Compound J, there was no increase in the levels of circulating immune complexes by the hepatic knockdown of C3 expression compared to CIC levels observed from PBS-treated control group.

### Example: 8: Effect of Compound J on C3 Expression in MRL/Ipr Mouse, a Lupus Nephritis Model

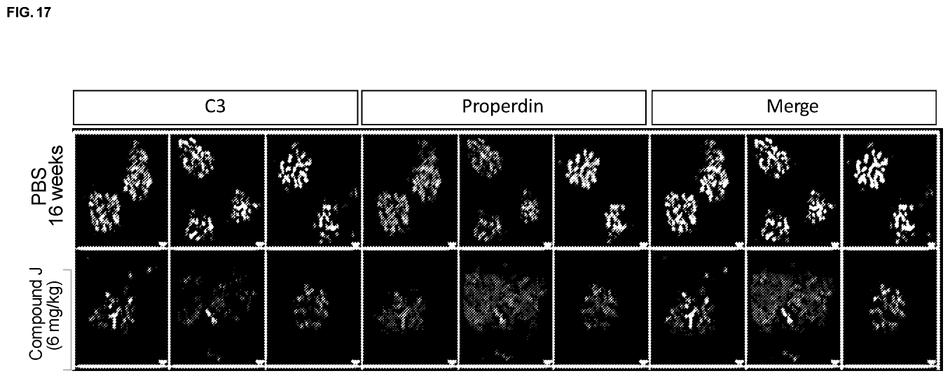

An MRL/Ipr lupus mouse model was used and treated with Compound J. The mice received multiple doses of 6 mg/kg of Compound J. FIG. 17 shows reduction of C3 glomerular deposition from kidneys of MRL/Ipr mice treated with multiple doses of 6 mg/kg of Compound J. A reduction of properdin deposits is also observed from the kidney samples of animals treated with subcutaneous doses of 6 mg/kg of Compound J every two weeks from the ages of 8 to 16 weeks as shown in FIG. 17.

### Example 9: Effect of Compound J on C3 Expression in Cfh^{-/-} Mice, a Complement Dysregulation Model

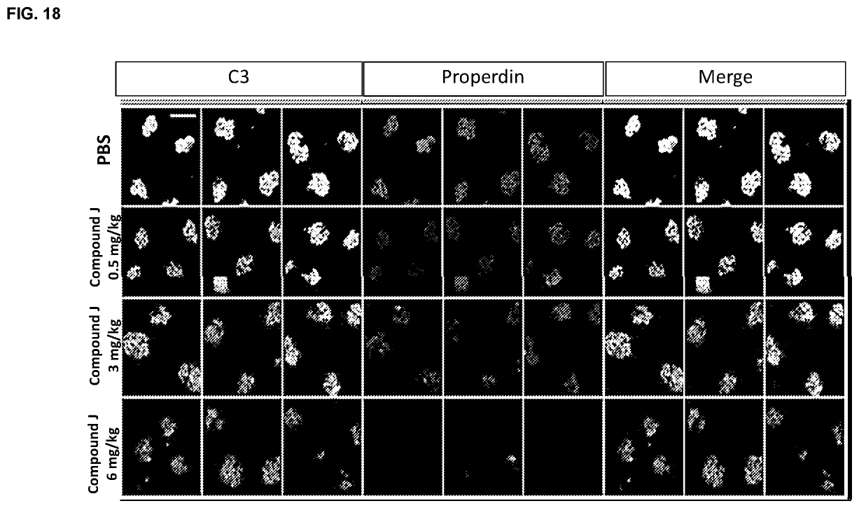

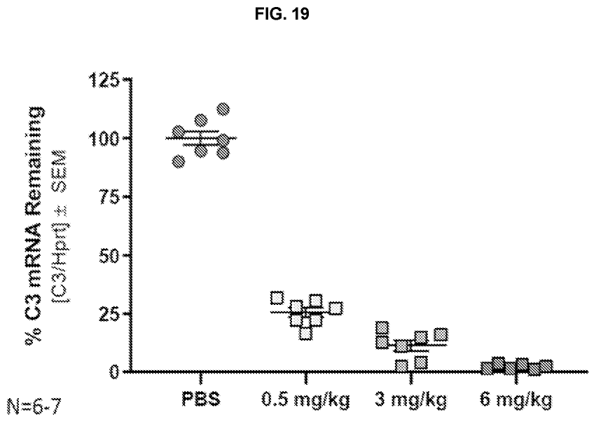

Mice deficient in complement factor H (Cfh^{-/-}) were administered four monthly doses of 0.5 mg/kg, 3 mg/kg, or 6 mg/kg of Compound J from the age of 4 months to 8 months. Kidneys from all treatment groups were collected 4 weeks after last dose of Compound J and immunofluorescence analysis were performed to visualize both C3 and properdin deposition in the glomeruli of CFH−/− treated animals. FIG. 18 shows a dose dependent reduction of C3 glomerular deposition from kidneys of CFH−/− mice treated with multiple increasing doses of Compound J. A reduction of properdin deposits was also observed from the kidney samples of animals treated with subcutaneous doses of Compound J every four weeks from the ages of 16 to 32 weeks of age as shown in FIG. 18. The percent of liver C3 mRNA was measured using RT-qPCR (FIG. 19). Treatment ablated C3 and properdin deposition in the kidney. In addition, the treatment with Compound J also normalized serum C5 levels in these mice (C5 consumption is a hallmark of complement dysregulation in this model).

### Example 10: Effect of Compound J on C3 Expression in a CAIA-Induced Arthritis Mouse Model

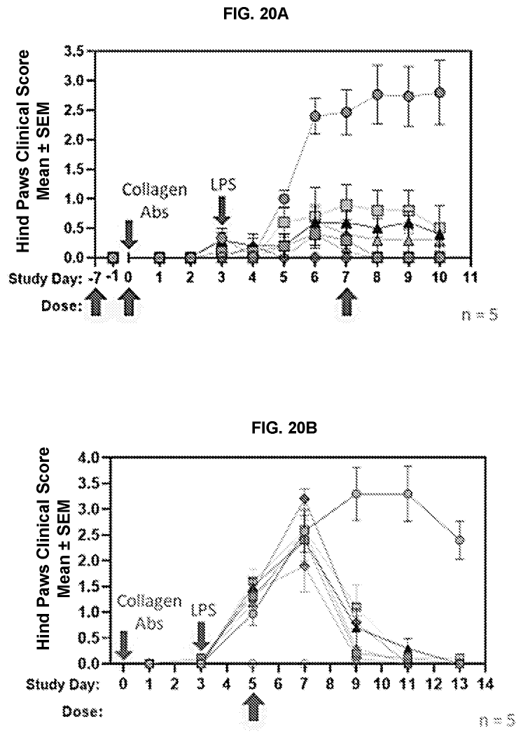

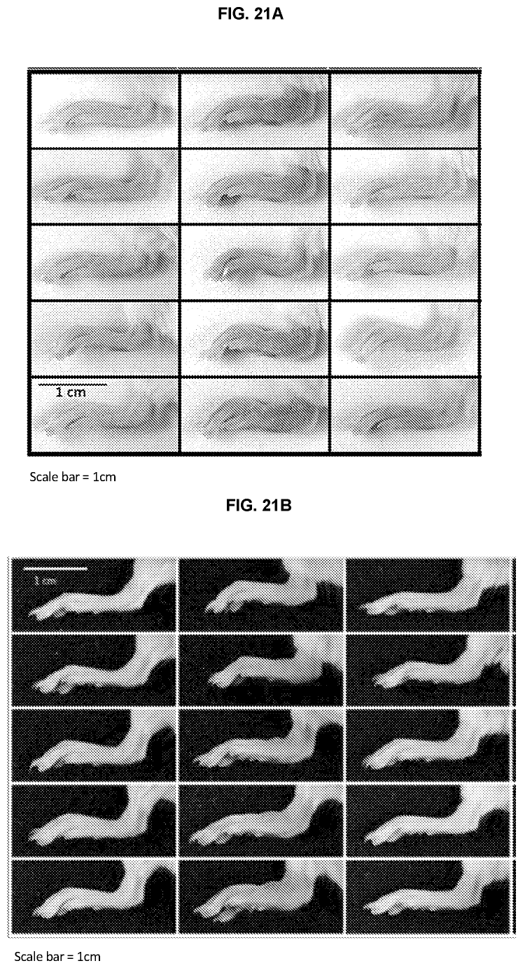

The effect of Compound J in treating symptoms related to arthritis were studied using a collagen antibody-induced arthritis (CAIA) induced arthritis mouse model, which is a simple model for rheumatoid arthritis. The CAIA-induced arthritis mouse model was generated by administering a collagen antibody to the mouse on day 0, followed by administration of an LPS booster on day 3. Compound J was tested in both preventative and therapeutic studies. Animals were dosed with 3 or 6 mg/kg of Compound J on day-7, for preventative study (FIG. 20A), or after disease onset on day 5, for therapeutic study (FIG. 20B). The hind paw inflammation was analyzed visually on day 10 and results from both preventative and therapeutic studies are shown in FIGS. 21A and 21B, respectively. Prophylactic treatment with Compound J prevented the swelling of hind paws, a characteristic hallmark of this model (FIG. 21A). Therapeutic treatment with Compound J completely reverted clinical disease manifestation after a single dose when compared to PBS-treated control animals (FIG. 21B).

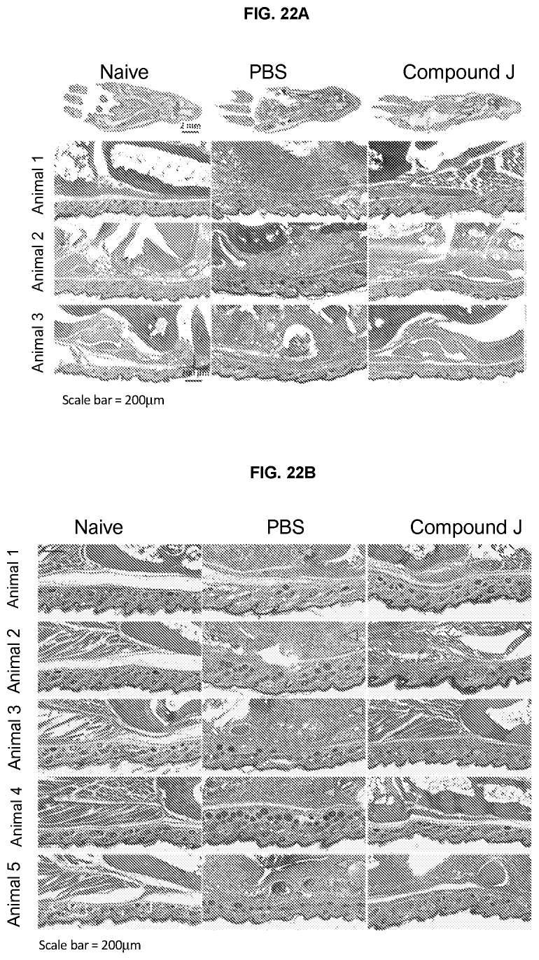

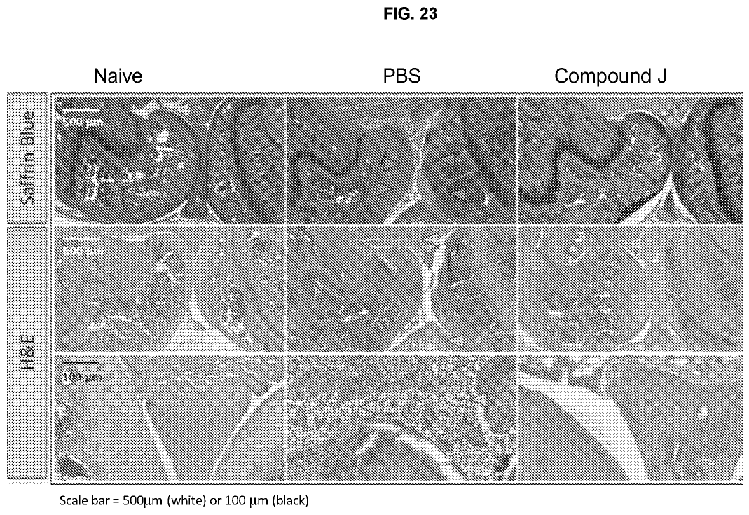

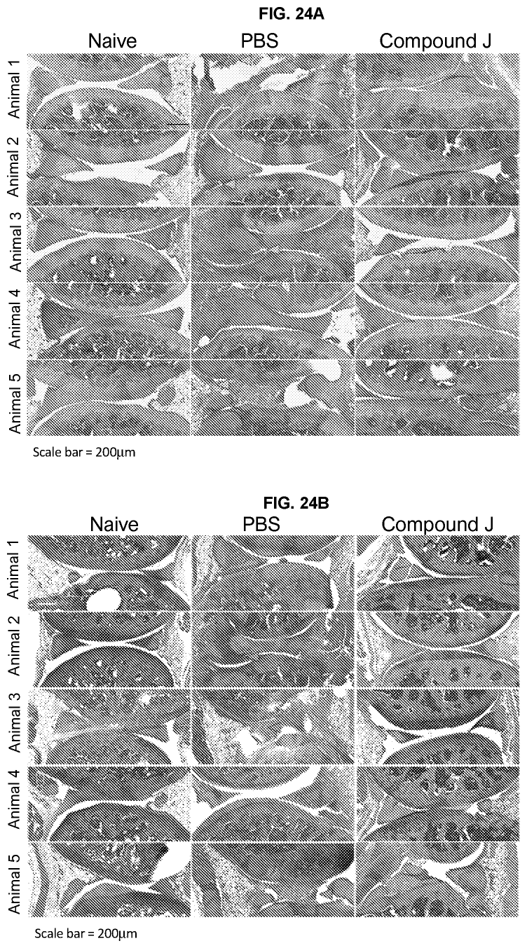

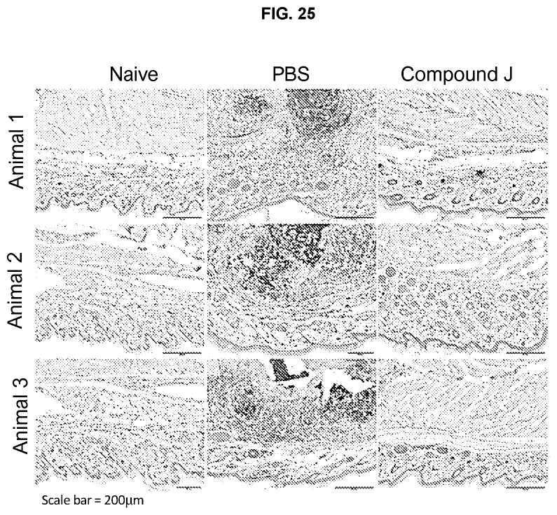

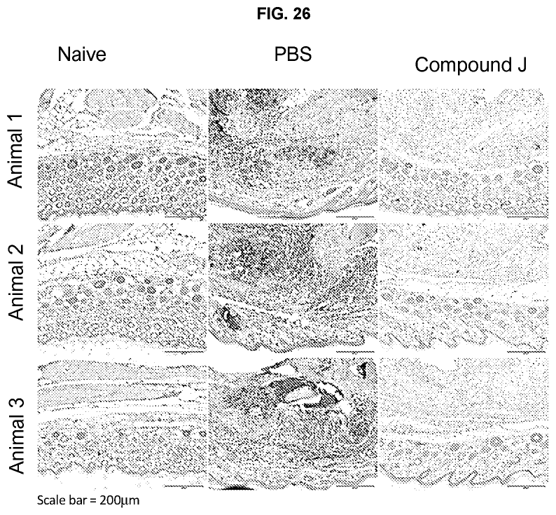

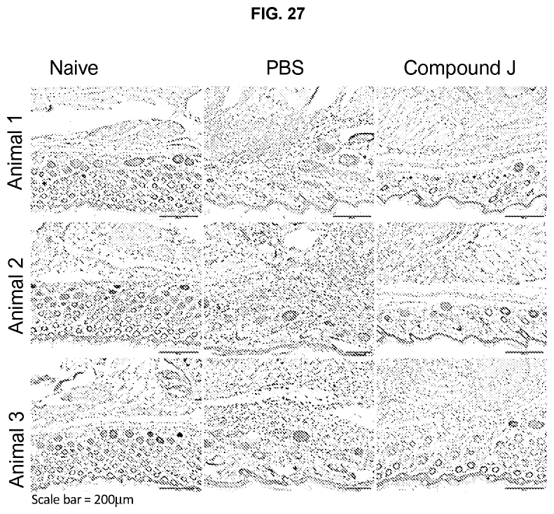

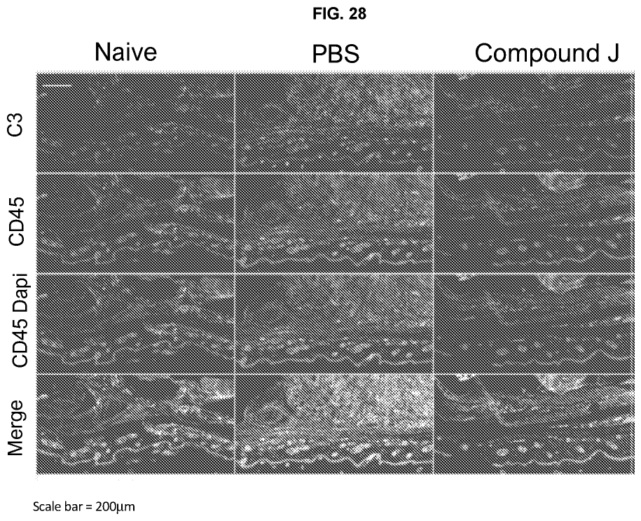

Hematoxylin and eosin (H&E) staining was performed on the biopsy of the hind paws and knees and shows a reduction of local mononuclear cells infiltration in mice that were treated with a single dose of 6 mg/kg of Compound J, either preventatively with 3 doses (FIG. 22A) or therapeutically with a single dose (FIGS. 22B and 24A respectively). Additionally, lymphocytes (CD45 positive cells), leukocytes (CD11b positive cells) and macrophages (F4/80 positive cells) marker staining were performed on biopsy samples as shown in FIGS. 25, 26, and 27, respectively, to show the reduction of local inflammation as a result of therapeutic treatment with 6 mg/kg of Compound J. Biopsy samples were also stained with Safranin O to visualize cartilage in the knees of the CAIA-induced arthritis mouse model. Animals treated with 6 mg/kg of Compound J showed a remarkable reduction in cartilage erosion in comparison to PBS-treated mice when treated preventatively (FIG. 23) or therapeutically (FIG. 24B). Experiments using in situ hybridization to C3 and CD45 mRNAs were performed on biopsy samples in order to assess complement expression at local sites of inflammation for CAIA-induced arthritic mice with and without treatment with 6 mg/kg Compound J, which is shown in FIG. 28. The hepatic knockdown of C3 with Compound J reduced the infiltration of lymphocytes (CD45 positive cells) and the local C3 mRNA expression with therapeutic treatment with Compound J in comparison to PBS-treated animals as a control group.

### Example 11: Effect of Compound J on C3 Expression in a Multiple Sclerosis Mouse Model

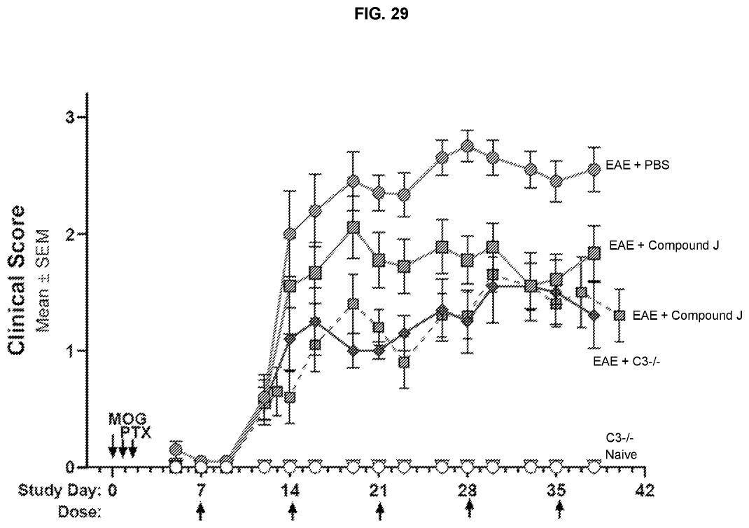

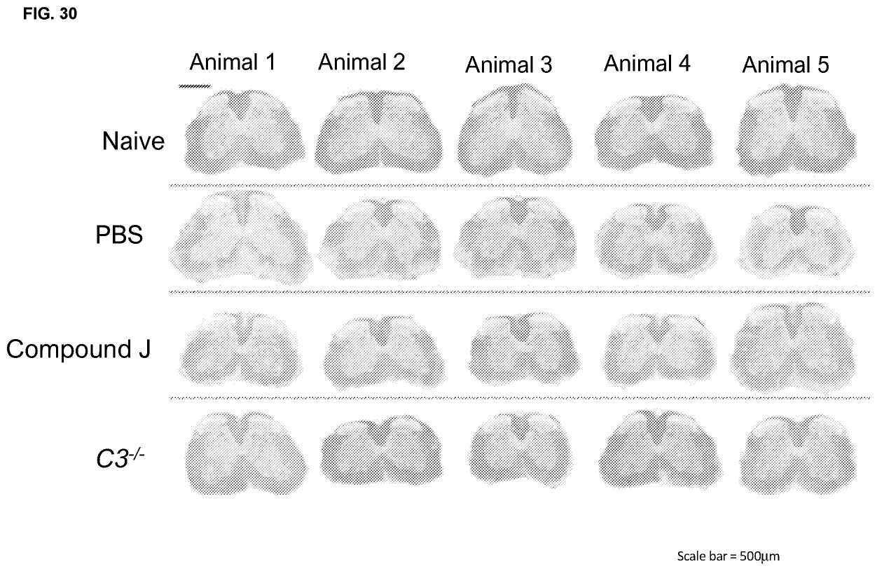

Myelin oligodendrocyte glycoprotein (MOG)-induced experimental autoimmune encephalomyelitis (EAE) mouse model, a model widely used to investigate the immune-mediated mechanism of neuroinflammation and demyelination was treated preventatively with a 6 mg/kg dose of Compound J (n=2 experiments). The liver C3 mRNA levels after treatment with Compound J as well as the C3 protein in serum was assessed using RT-qPCR and a mouse C3 ELISA assay respectively as shown in FIGS. 31A and 31B. Likewise, the percent of C3 mRNA remaining after treatment with Compound J as well as the amount of C3 in serum of MOG-induced EAE mice after being treated with a dosage of 6 mg/kg of Compound J in comparison to C3 deficient mice and MOG-induced EAE C3 deficient mouse strain treated with PBS was assessed. The hepatic knockdown of C3 with Compound J reduced the severity of the disease in both experiments performed (FIG. 29). The reduction of severity observed with liver knockdown by Compound J treatment was similar to the clinical observation in C3 deficient animals (global knockout).

Lumbar spinal cord samples were also obtained from MOG-induced EAE mice treated with Compound J. Luxol fast blue staining along with H&E staining was performed on spinal cord samples in order to visualize myelination as well as mononuclear cell infiltration as shown in FIG. 30. Luxol fast blue spinal cord samples were compared between disease animals treated with 6 mg/kg of Compound J, PBS, and C3 deficient mouse as shown in FIG. 30. MOG-induced animals treated with Compound J showed a reduction of the de-myelination and prevention of immune cell infiltration similar to the levels observed in MOD-induced EAE C3-deficient mice.

Similar results were observed in a proteolipid protein (PLP)-induced EAE model. Disease severity was reduced with C3 siRNA treatment but not sufficiently to prevent relapse, a feature of this animal model.

### Example 12: Murine Absorption, Distribution, Metabolism, and Excretion (ADME) Study

A pharmacokinetic and biodistribution study was performed in male CD-1 mice administered PBS (n=18) or 3, 10, or 100 mg/kg of Compound A via single subcutaneous (SC) injection (n=39/cohort), or 3 mg/kg via a single intravenous (IV) injection (n=36). Bioavailability was approximately 18% based on a comparison of AUCₗₐₛₜ after IV versus SC dose at 3 mg/kg. However, liver exposures were similar between the 3 mg/kg IV and SC cohorts. Plasma exposure compared to the 3 mg/kg dose group increased roughly in a dose proportional manner for the 10 mg/kg group and greater than dose-proportional manner for the 100 mg/kg group. Liver exposure based on Cₘₐₓ and AUCₗₐₛₜ, increased approximately in a dose proportional manner at 10 mg/kg and in a less than dose proportional manner at 100 mg/kg compared to the 3 mg/kg dose group indicative of a saturation of the distribution process to the liver. The elimination half-life in liver ranged from 2.1 to 4 days.

### Example 13: Platelet Activation of Compound A

An assessment of platelet activation in human whole blood stimulated with Compound A did not induce platelet activation. Whole blood samples (5 male and 5 female donors) were stimulated with PBS or Compound A at 10, 100, 200, or 300 μg/mL.

### Example 14: Tolerability of Compound A in Cynomolgus Monkeys

The tolerability of dosing of Compound A was evaluated in a study in naïve male and female cynomolgus monkeys. Animals were administered SC doses of phosphate buffered saline (PBS), n=12) or Compound A at 1.5 mg/kg (low) or 3 mg/kg (high) dose levels on day 0 (n=6/cohort), and liver biopsies were performed on day 21 to determine the level of hepatic C3 knockdown. Based on this analysis, the dosing levels on days 28, 56 and 84 were adjusted to 3 mg/kg (for the low dose) or 6 mg/kg (for the high dose) to try to approximate 75% and 90% C3 mRNA knockdown, respectively. Blood samples were collected on days −21, −7, −3, 0, 28, 56 and 112 and viral, bacterial and parasitic testing of a total of 28 pathogens was conducted pre-study, on day 56 and at necropsy to monitor for potential reactivation of latent viruses and/or infections by serology and blood or fecal PCR. Terminal necropsy for assessment by a clinical pathologist was conducted to determine potential evidence of infection. An interim measurement of circulating C3 protein levels in serum along with CBC, coagulation, clinical chemistry, and urinalysis was also performed. By day 49 following dosing with either the low or high doses of Compound A, hepatic C3 mRNA knockdown of approximately 75 or 80%, respectively, was achieved, with an approximately 80% reduction in the circulating C3 protein levels. These reductions in hepatic C3 mRNA and C3 protein levels were sustained at all timepoints evaluated through day 112, when the study was terminated. There were no gross or microscopic findings at necropsy and no unscheduled mortalities. Body weight, liver function tests, blood cell counts, blood chemistry, and lipid metabolism parameters were not affected by chronic Compound A treatment and there was no evidence of increased pathogenic parasitic, bacterial or viral infection in the treated monkeys compared with a cohort dosed with PBS.

### Example 15: Tolerability of Compound J in CD-1 and NZB/W F1 Mice

The tolerability of chronic dosing of Compound J was evaluated in CD-1 and NZB/W F1 mice. CD-1 mice were administered a total of 4 monthly SC doses of PBS or Compound J (1 or 100 mg/kg), sacrificed one month after the final dose and evaluated by a blinded pathologist for evidence of viral or bacterial infection and histological changes. No treatment-associated histopathological changes or increased infections were observed. Similarly, NZB/W F1 mice were administered 1 or 6 mg/kg Compound J every 4 weeks from 28 to 40 weeks of age with terminal sacrifice at 44 weeks of age or from 24 to 36 weeks of age with terminal sacrifice at 40 weeks of age. No evidence of increased infection was detected by serological and PCR testing for a panel of viruses, bacterial pathogens and parasites, and terminal necropsies evaluated by a veterinary pathologist blinded to the treatment groups did not identify any treatment-associated changes.

### Example 16: Safety Pharmacology Study of Compound A in Cynomolgus Monkeys

Compound A was evaluated in a subcutaneous safety pharmacology study in cynomolgus monkeys. Four animals were administered PBS or increasing single doses of Compound A (at 30, 100 and 300 mg/kg dose levels) once every 7 days, with the same four animals used for each dosing occasion. Safety pharmacology, including assessment of cardiovascular (e.g., ECG, blood pressure, heart rate, etc.), respiratory (respiratory rate) and neurological (functional observational battery) endpoints, as well as clinical assessments, were performed during this study. There were no cardiovascular or respiratory effects observed at any dose level. There were no neurological effects observed at 30 or 100 mg/kg Compound A. At 300 mg/kg, clinical observations of slight to mild tremors (limbs or whole body) were noted in 3 animals at 4 and 24 hours post-dose. The neurological observations observed at 300 mg/kg were considered adverse. Therefore, the no-observed-adverse-effect level (NOAEL) was determined to be 100 mg/kg. Additionally, genetic toxicology assessments which included in vitro micronucleus assay and in vitro bacterial reverse mutation assay were negative for inducing micronuclei and for mutagenic activity, respectively.

### Example 17: PK/PD Study of Compound A in Cynomolgus Monkeys

A single dose PK/PD study was performed in cynomolgus monkeys. Animals received a SC dose of PBS (control) or a single SC or IV dose of 3 mg/kg Compound A on day 0 (n=5 per group). Compound A concentrations in plasma, urine, and tissues were evaluated. Liver biopsies were performed on days 2, 35, 70, 112, 158 and 252 to evaluate liver C3 mRNA levels (the primary pharmacodynamic marker). Circulating C3 protein levels and complement functional activity were additional PD markers that were assessed throughout the study.

Concentrations of Compound A were determined in plasma, liver, and urine using a qualified hybridization-based anion exchange high-performance liquid chromatography with fluorescence detection (AEX-HPLC-FD) method. The reduction of complement component 3 (C3) mRNA expression in monkey liver was measured using real time quantitative polymerase chain reaction (RT-qPCR). The C3 protein in monkey serum was measured using ELISA and the complement functional activity (classical pathway, mannose-binding lectin pathway (MBL), and alternative pathway) was measured using the WIESLAB® assay.

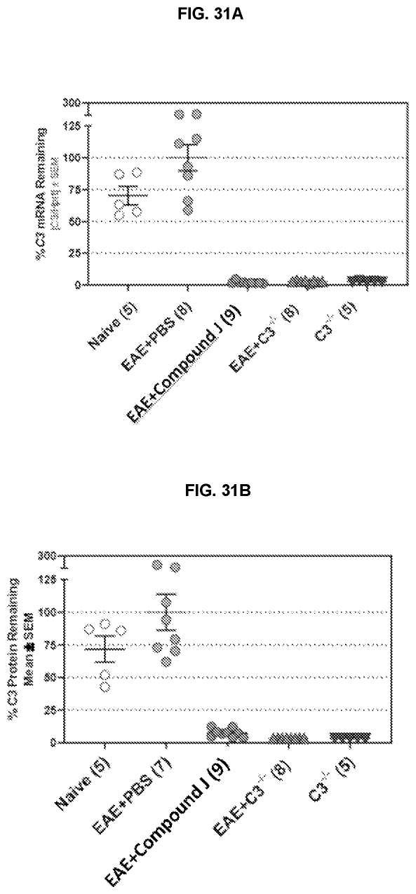

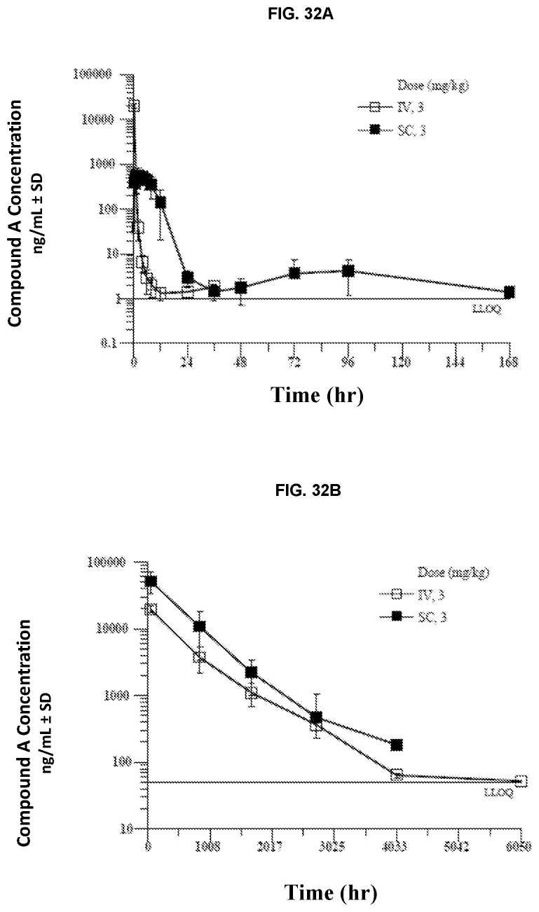

Plasma concentrations of Compound A over time were used to generate a noncompartmental PK profile for individual animals (group mean is plotted in FIG. 32A). The plasma Tₘₐₓ range following SC administration was 1 to 6 hours and the Tₘₐₓ for IV administration was 0.25 hour (first timepoint collected) for all animals. Plasma concentrations of Compound A decreased in a biphasic manner, with a slower distribution phase for the SC route compared to the IV route. The biphasic decrease is indicative of an initial rapid distribution phase, primarily to the liver, followed by a slower elimination phase. Plasma half-lives were reported for 1 of 5 animals in the SC group (2.51 hours) and 2 of 5 animals in the IV group (mean=1.21 hours). The half-lives for the remaining animals were not reported as the acceptance criteria for the terminal phase rate constant were not met. The bioavailability of Compound A was approximately 28.5% for the SC dose relative to the IV dose based on AUCₗₐₛₜ.

Liver concentrations of Compound A over time were used to generate a noncompartmental PK profile for individual animals (group mean is plotted in FIG. 32B). Half-lives in the liver were calculated for all 5 animals in both the SC and IV groups and ranged from 13 to 20 days and 20 to 33 days, respectively.

The total amount of Compound A excreted in the urine within each collection time interval by each animal was used to calculate the total drug excreted and % drug excreted. Mean urinary excretion of Compound A was 4.3% and 4.8% for the SC and IV groups, respectively.

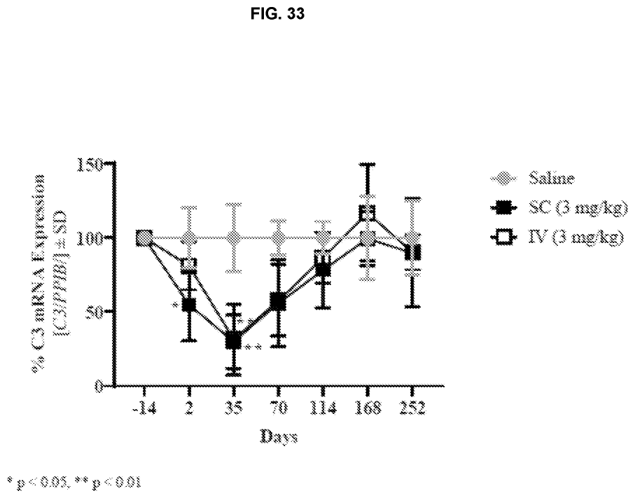

Following a single SC or IV administration of 3 mg/kg of Compound A, reduction of C3 mRNA expression in monkey liver was observed on Day 2 (SC only) and reached maximum reduction (approximately 70% for both dose routes) on Day 35 postdose (FIG. 33). Gradual recovery of C3 mRNA expression over time was observed with a return to near baseline levels by Day 114. This recovery was maintained up to Day 252 for both routes of administration.

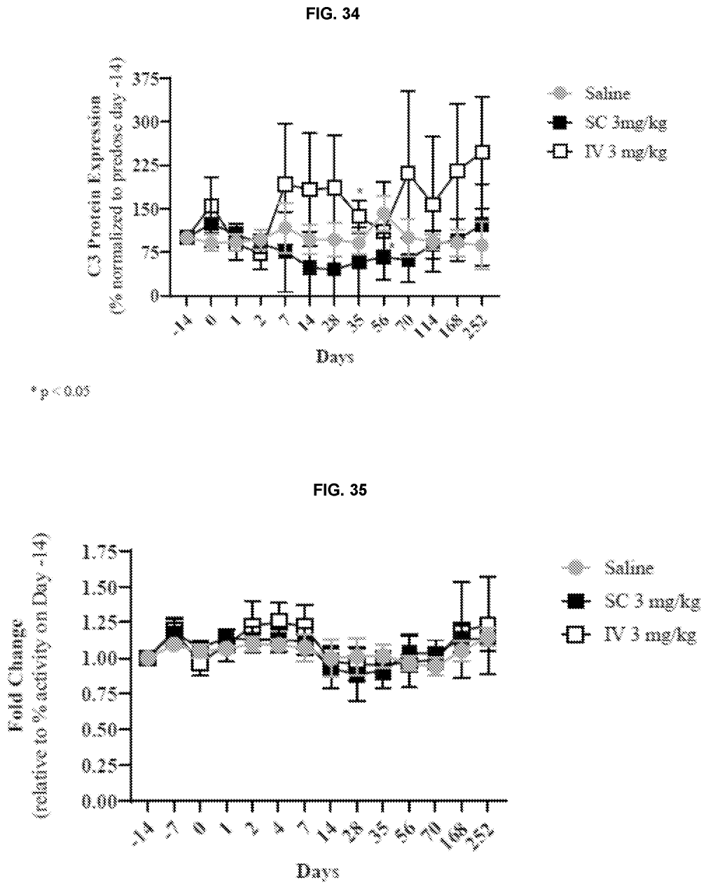

After a single 3 mg/kg SC administration of Compound A, reduction in serum C3 protein was observed from Day 7 to Day 70 (FIG. 34). Maximum mean C3 protein levels in serum decreased by 54.7% at 28 days postdose and recovered to predose levels by Day 168. Relative to controls, there was no reduction of the serum C3 protein levels in animals administered a single 3 mg/kg IV dose of Compound A.

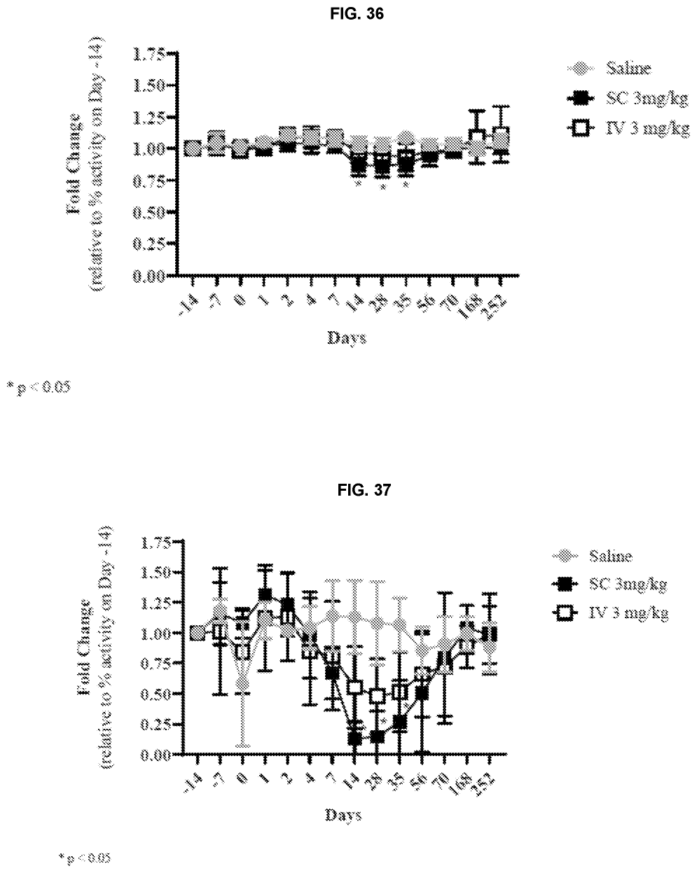

There was no effect on classical complement in either the SC or IV group relative to controls (FIG. 35). The functionality of the lectin and alternative pathways was reduced relative to controls in the SC group on Days 14, 28, and 35. Functionality of the lectin pathway was reduced minimally (approximately 13%) on all three days (FIG. 36), whereas functionality of the alternative pathway was reduced by 87%, 85%, and 73% on Days 14, 28, and 35 (FIG. 37), respectively. Pathway functionality returned to baseline levels by Day 70 for the lectin pathway and Day 168 for the alternative pathway.

Following a single 3 mg/kg SC administration of Compound A, maximum mRNA reduction was 70% on Day 35, maximum reduction of C3 protein was 54.7% on Day 28, and maximum reduction of alternative pathway activity was >87% on Day 14. mRNA expression recovered to baseline levels by Day 168, C3 protein expression returned to baseline by Day 28, and alternative pathway functionality was recovered by Day 168.

### Example 18: Toxicology Studies in CD-1 Mice and Cynomolgus Monkeys

A 6-month toxicology study in mice and a 9-month toxicology study in cynomolgus monkeys were performed. The ranges of dose levels in these studies were selected to achieve at least a 10-fold exposure multiple over the expected exposure of the highest intended clinical dose.

In the murine study, the potential toxicity of repeat-dose (every 4 weeks; 7 doses) SC administration of PBS or Compound A (30, 100, or 300 mg/kg) in CD-1 mice and the potential reversibility of any findings following an 8-week recovery period were evaluated. Ten male and 10 female mice per dosing cohort were evaluated during the dosing portion of the study, and 6 males and 6 females were maintained during the recovery phase. In addition, the toxicokinetic characteristics of Compound A were determined in a sub-study (n=111). Compound A dosed at levels of 30, 100, and 300 mg/kg was well tolerated with no Compound A-related mortality or adverse findings. Clinical pathology findings included minimally increased alanine aminotransferase and minimally decreased triglycerides at Day 171 with complete or partial reversibility evident at the end of the recovery period. Compound A-related non-adverse microscopic findings included minimal or mild mixed cell inflammation of the liver, hepatocellular karyocytomegaly, increased mitoses, and oval cell hyperplasia at the terminal euthanasia with minimal or mild increased mitoses and hepatocellular karyocytomegaly still present at the recovery euthanasia. Based on these results, the NOAEL was considered to be 300 mg/kg, which corresponded to mean AUCₗₐₛₜ values of 760,000 and 543,000 hr*ng/ml and mean Cₘₐₓ values of 160000 and 96400 ng/mL for males and females, respectively, on Day 169.

In the monkey study, the potential toxicity of repeat SC dosing (0, 30, 100, 300 mg/kg every 4 weeks for a total of 10 doses) of Compound A, and the reversibility, persistence, or delayed occurrence of any effects after an 8-week recovery period were evaluated. Four male and 4 female monkeys were included in each dose cohort in the main dosing study, and 2 males and 2 females were included in the 2-month recovery period. In addition, the toxicokinetic and PD characteristics of Compound A were determined. Repeat SC administration of Compound A for 9 months to cynomolgus monkeys was well tolerated. Repeat-dose administration of Compound A produced no test article-related changes in the following parameters: clinical observations, neurologic examinations, ophthalmic examinations, body weights, qualitative food consumption, hematology, clinical chemistry, urinalysis, cytokines (i.e., MCP-1, TNF-α, IL-8, IL-1RA, G-CSF, IFN-γ, IL-1β, and IP-1), complement factors Bb and C3a, gross pathology, or organ weights during the dosing phase of the study. In addition, there were no early mortalities during this study, and all animals survived to the scheduled necropsies. Compound A-related non-adverse increases in fibrinogen occurred at ≥100 mg/kg/dose. At ≥30 mg/kg/dose, there were non-adverse microscopic findings (vacuolated/granular macrophages) in the liver as well as in multiple other tissues. Based on the findings observed, the NOAEL was determined to be 300 mg/kg/dose, with associated AUCₗₐₛₜ of 1330000 hr*ng/ml and Cₘₐₓ of 70900 ng/ml (males and females combined, Day 253).

Genetic toxicology assessments, which included in vitro micronucleus assay and in vitro bacterial reverse mutation assay, were negative for mutagenic activity and for inducing micronuclei, respectively.

Based on the totality of data, the NOAEL is considered to be 300 mg/kg. The neurological clinical signs (e.g., tremors) that were noted in the study with weekly increasing doses were not noted in the 9-month monkey toxicology study with monthly dosing.

### Example 19: Effect of Compound B on C3 Expression in Cynomolgus Monkeys, an AMR Model

A pharmacology study in four sensitized cynomolgus monkeys receiving kidney allografts is performed. In this study, animals receive a SC dose of Compound B once every 4 weeks over a total of 4 months.

### Example 20: Dose Ranging Study in Humans

A single ascending dose (SAD) study in healthy volunteers is performed, combined with a multiple ascending dose (MAD) cohort in patients with complement driven diseases for a First-In-Human (FIH) clinical study. Healthy volunteers and patients receive prophylactic vaccinations against Neisseria meningitides types A, C, W, Y, and B, Streptococcus pneumoniae, and Haemophilus influenzae type B prior to receiving Compound A.

### Example 21: Treating Multiple Sclerosis with Compound A in Humans

A subject suffering from multiple sclerosis is treated with a pharmaceutical composition containing Compound A (e.g., in a dose amount of about 0.01 mg/kg-50 mg/kg bodyweight of the subject). The subject is administered the composition at a frequency of about once a week, for example, by subcutaneous injection, for a period of about 12 months or longer (e.g., until symptoms resolve or stabilize). Approximately once a month, the subject's symptoms and serum C3 levels are evaluated by a clinician to assess the efficacy of Compound A. The subject's serum C3 is quantified using a blood serum sample and can be compared to the amount of C3 protein found in the serum of the subject prior to being administered Compound A or relative to a control amount of C3 protein or the amount of C3 protein present in a serum sample from a normal subject (e.g., a disease-free subject). Treatment with Compound A is determined to be effective if the amount of C3 protein in serum decreases, i.e., by at least 10%, in comparison to the amount of C3 protein in serum prior to treatment with Compound A. Additionally, the subject's symptoms associated with multiple sclerosis, such as blurry vision, slurred speech, dizziness, tingling, lack of coordination, and unsteady gait, can be assessed by a clinician to evaluate if there is a decrease in any or all of the symptoms a subject is experiencing in comparison to the symptoms the subject was experiencing prior to being administered Compound A, and/or in comparison to a placebo control subject.

### Example 22: Treating Arthritis with Compound A in Humans

A subject diagnosed with arthritis is treated with a pharmaceutical compound containing Compound A (e.g., in a dose of about 1.5 mg/kg). The subject is administered the composition at a frequency of about once a month, for example, by subcutaneous injection, for a period of about 6 months or longer (e.g., until symptoms resolve or stabilize). The subject is evaluated (e.g., by assessing the subject's symptoms and/or serum C3 levels) by a clinician to assess the efficacy of Compound A every one or two months. The subject's serum C3 is quantified using a blood serum sample from the subject and is compared to the amount of C3 protein found in the serum of the subject prior to being administered Compound A or relative to a control amount of C3 protein or the amount of C3 protein present in a serum sample from a normal subject (e.g., a disease-free subject), and/or compared to the amount of C3 protein present in a serum sample from a placebo-treated patient. Treatment with Compound A is determined to be effective if the amount of C3 protein in serum decreases by at least 10% in comparison to the amount of C3 protein in serum prior to treatment with Compound A. Additionally, the subject's symptoms associated with arthritis, including pain, stiffness, swelling, redness, and a decreased range of motion, can be assessed by a clinician to evaluate if there is a decrease in any or all of the symptoms a subject is experiencing in comparison to the symptoms the subject was experiencing prior to being administered Compound A.

### Example 23. C3 Assessment Assays

Various assays including assessment of Compound A in plasma or tissue, WIESLAB® complement functional activity assays, assessment of circulating C3, C3 mRNA expression levels, and pharmacokinetic assays may be used as described herein to characterize the effect of Compound A on C3 levels.

### Pharmacokinetic Assays of Compound A

Concentrations of Compound A or Compound J in plasma of mouse and monkey were measured Plasma samples (blanks, unknowns, standards, and QC samples) are enzymatically treated followed by hybridization with the peptide nucleic acid (PNA) probe that has sequence complementarity to the antisense strand of Compound A or Compound J. Samples are injected into a high-performance liquid chromatography (HPLC) equipped with a fluorescence detector. Chromatographic separation was performed using a gradient system on Shimadzu Prominence systems using DNAPAC™ PA200 analytical columns. The fluorescence detector monitored signals from 436 nm (Ex) to 484 nm (Em). To check the retention time of the metabolites, reference samples of individuals and mixtures were prepared and injected. The peaks of Compound A and its expected metabolites were successfully separated. Quantitation of Compound A or Compound J in monkey or murine plasma, respectively, was performed using linear regression. This assay was used, for example, in Examples 6, 12, and 17 as described above.

### WIESLAB® Complement Functional Activity Assay (CCP, CAP, CLP)

The complement classical pathway (CCP), CAP, and complement lectin pathway activities were evaluated using a WIESLAB® Complement System Screen assay, using labeled antibodies specific for a neoantigen to detect the human terminal complement complex (C5b-9) complex produced as a result of complement activation. The assay is also able to detect cynomolgus monkey C5b-9. The amount of neoantigen generated was proportional to the level of functional activity of the individual pathways. Wells in the assay's microtiter strips were coated with specific activators of the classical, or the alternative, or the lectin pathways. Monkey serum samples were diluted in diluent containing a blocker which ensures that only the respective pathway were activated. The wells were washed and C5b-9 was detected with a specific alkaline phosphatase-labeled antibody to the neoantigen expressed. The amount of complement activation correlated with the color intensity measured by absorbance at 405 nm. The value for the positive control provided in the test kit was defined as 100% complement activation. All measured values were expressed as percent (%) complement activity, determined as follows:

[(Sample−negative control)/(positive control−negative control)]*100

This assay was used, for example, in Example 17 above.

### Circulating C3 Protein Level

Assessment of cynomolgus circulating C3 protein levels was evaluated using a Human Complement C3 enzyme-linked immunosorbent assay (ELISA) Kit (cat #Ab108823, Abcam, Cambridge, UK) designed for the quantitative measurement of Complement C3 concentrations in human serum. Since there is cross-reactivity with monkey C3, this kit was also used for the determination of circulating C3 protein in cynomolgus serum samples. A complement C3 specific antibody was precoated onto 96-well plates and blocked. Standards or test samples were added to the wells and subsequently a complement C3 specific biotinylated detection antibody was added, followed by wash buffer. Streptavidin-Peroxidase conjugate was added and unbound conjugates were removed with wash buffer. Tetramethylbenzidine (TMB) was used to visualize the Streptavidin-Peroxidase enzymatic reaction. TMB was oxidized by Streptavidin-Peroxidase to produce a blue color product that changes into yellow after adding acidic stop solution. The density of yellow coloration was directly proportional to the amount of complement C3 captured in the plate. Back-calculated concentration of the sample was determined by the curve fitting regression program generated by the calibration standards. This assay was used, for example, in Example 14 and 17 above.

C3 mRNA Expression Levels

Assessment of C3 mRNA expression was determined in cynomolgus monkey liver samples using a Multiplex Relative Quantitation Real-Time Reverse Transcriptase PCR Assay. mRNA was isolated from frozen liver tissue, followed by mRNA quantitation and transcription into complementary DNA (cDNA). The cDNA was used as the template for the qPCR reaction to measure C3 mRNA levels with normalization to peptidyl-prolyl cis-trans isomerase B (PPIB). The degree of C3 mRNA in the treated groups was calculated as the percent of expression (normalized to PPIB mRNA levels) relative to untreated or the pre-dose group, where C3 mRNA expression in the control group is set at 100%.

### Pharmacokinetic Assays

Concentrations of Compound A in human plasma is measured using a HPLC-FD analytical method. Plasma samples (blanks, unknowns, standards, and quality control [QC] samples) are enzymatically treated with Proteinase K, followed by hybridization with the PNA probe that had sequence complementarity to the antisense strand of Compound A. Samples are injected into an HPLC equipped with a fluorescence detector. Chromatographic separation is performed using a gradient system on Shimadzu Prominence systems using DNAPAC™ PA200 analytical columns. A fluorescence detector monitors signals from 436 nm (Ex) to 484 nm (Em). LC gradient conditions are adjusted and determined based on the retention time of the potential metabolites of Compound A. To evaluate the retention time of the metabolites, reference samples of individuals and mixtures are prepared and injected. The peaks of Compound A and the metabolites are separated. Quantitation of Compound A in human plasma is performed using linear regression.

### Antidrug Antibody Assay

The antidrug antibody (ADA) assay for Compound A in human serum is under development and is planned to be performed using an electrochemiluminescence (ECL) bridging assay. Positive controls (PCs) are being generated from rabbits immunized against an immunogenic cocktail consisting of keyhole limpet hemocyanin (KLH)-conjugated Compound A and KLH-conjugated oligonucleotides of various lengths corresponding to modified Compound A sequences. The PCs, negative controls (NCs), and study samples will be subjected to an acid dissociation step at ambient room temperature then added to a plate containing TRIS, biotin-Compound A, and ruthenium-labeled Compound A, enabling formation of bridging complexes between the labeled Compound A and the Compound A antibodies present in the sample. After incubation, NC, PC, and study samples will be transferred to a streptavidin-coated plate and incubated in the dark for 1 hour during which drug binds to the plate capturing the ADA bridging complex. The plate is then washed, and an Meso Scale Discovery® (MSD®) read buffer is added to generate an ECL signal which is directly proportional to the amount of ADA present in the sample. The ADA assay will be validated prior to evaluation of clinical samples.

### OTHER EMBODIMENTS

All publications, patents, and patent applications mentioned in this specification are herein incorporated by reference to the same extent as if each independent publication or patent application was specifically and individually indicated to be incorporated by reference. While particular embodiments are herein described one of skill in the art will appreciate that further modifications and embodiments are encompassed including variations, uses or adaptations generally following the principles described herein and including such departures from the present disclosure that come within known or customary practice within the art and may be applied to the essential features hereinbefore set forth, and follows in the scope of the claims.

1. An RNAi oligonucleotide, or a pharmaceutically acceptable salt thereof, for reducing complement component C3 (C3) expression, the oligonucleotide comprising a sense strand and an antisense strand, wherein the sense strand and the antisense strand form a duplex region, wherein the antisense strand comprises a region of complementarity to a C3 mRNA target sequence of SEQ ID NO: 13 or 14, and wherein the region of complementarity is at least 15 contiguous nucleotides in length.

2. The RNAi oligonucleotide of claim 1, or a pharmaceutically acceptable salt thereof, wherein:
(a) the sense strand is 15 to 50 nucleotides in length, and/or
(b) the antisense strand is 15 to 30 nucleotides in length.

3-6. (canceled)

7. The RNAi oligonucleotide of claim 1, or a pharmaceutically acceptable salt thereof, wherein the region of complementarity is at least 19 contiguous nucleotides in length, optionally at least 20 nucleotides in length.

8. The RNAi oligonucleotide of claim 1, or a pharmaceutically acceptable salt thereof, wherein the 3′ end of the sense strand comprises a stem-loop set forth as S1-L-S2, wherein S1 is complementary to S2, and wherein L forms a loop between S1 and S2 of 3-5 nucleotides in length.

9-10. (canceled)

11. The RNAi oligonucleotide of claim 8, or a pharmaceutically acceptable salt thereof, wherein:
(a) L is a tetraloop, wherein the tetraloop comprises the nucleic acid sequence of SEQ ID NO: 8; and/or
(b) the stem-loop comprises a nucleic acid sequence having at least 85% identity to SEQ ID NO: 7; and/or
(c) the stem-loop comprises a nucleic acid sequence having up to 1, 2, or 3 substitutions, insertions, or deletions relative to SEQ ID NO: 7.

12-21. (canceled)

22. The RNAi oligonucleotide of claim 1, or a pharmaceutically acceptable salt thereof, wherein the oligonucleotide comprises at least one modified nucleotide.

23-24. (canceled)

25. The RNAi oligonucleotide of claim 22, or a pharmaceutically acceptable salt thereof, wherein the modified nucleotide comprises a 2′-modification, wherein the 2′-modification is a modification selected from 2′-aminoethyl, 2′-fluoro, 2′-O-methyl, 2′-O-methoxyethyl, and 2′-deoxy-2′-fluoro-β-d-arabinonucleic acid.

26-28. (canceled)

29. The RNAi oligonucleotide of claim 25, or a pharmaceutically acceptable salt thereof, wherein:
(a) at least one of nucleotides 1-7, 11-27, and 31-36 of the sense strand and one or more, or all, of nucleotides 1, 6, 8, 9, 11-13, and 15-22 of the antisense strand are modified with a 2′-O-methyl, such as a 2′-O-methyl ribonucleoside; or
(b) wherein at least one of nucleotides 3, 8, 9, 10, 11, 12, 13, and 17 of the sense strand and one or more, or all, of nucleotides 2, 3, 4, 5, 7, 8, 10, 12, 14, 16, and 19 of the antisense strand are modified with a 2′-fluoro, such as 2′-fluoro deoxyribonucleoside.

30-37. (canceled)

38. The RNAi oligonucleotide of claim 1, or a pharmaceutically acceptable salt thereof, wherein:
(a) the oligonucleotide comprises at least one modified internucleotide linkage; and/or
(b) there is no internucleotide linkage between the sense strand and the antisense strand; and/or
(c) the 4′-carbon of the sugar of the 5′-nucleotide of the antisense strand comprises a phosphate analog; and/or
(d) the RNAi oligonucleotide comprises a uridine at the first position of the 5′ end of the antisense strand.

39-46. (canceled)

47. The RNAi oligonucleotide of claim 1, or a pharmaceutically acceptable salt thereof, wherein at least one nucleotide of the oligonucleotide is conjugated to one or more targeting ligands, optionally wherein each targeting ligand comprises a N-acetylgalactosamine (GalNAc) moiety.

48-49. (canceled)

50. The RNAi oligonucleotide of claim 47, or a pharmaceutically acceptable salt thereof, wherein the GalNAc moiety is a monovalent GalNAc moiety, a bivalent GalNAc moiety, a trivalent GalNAc moiety or a tetravalent GalNAc moiety.

51-60. (canceled)

61. The RNAi oligonucleotide of claim 1, or a pharmaceutically acceptable salt thereof, wherein the sense strand and antisense strands comprise nucleotide sequences selected from the group consisting of:
(a) SEQ ID NOs: 1 and 3, respectively, and
(b) SEQ ID NOs: 4 and 6, respectively.

62-63. (canceled)

64. The RNAi oligonucleotide of claim 1, or a pharmaceutically acceptable salt thereof, wherein:
(a) the sense strand comprises a nucleotide sequence as set forth in SEQ ID NO: 37 and the antisense strand comprises a nucleotide sequence as set forth in SEQ ID NO: 38; or
(b) the sense strand comprises a nucleotide sequence as set forth in SEQ ID NO: 39 and the antisense strand comprises a nucleotide sequence as set forth in SEQ ID NO: 40.

65. (canceled)

66. A pharmaceutical composition comprising the RNAi oligonucleotide of claim 1, or a pharmaceutically acceptable salt thereof, and a pharmaceutically acceptable carrier, excipient, or diluent.

67. A method of treating a disease mediated by complement pathway activation or dysregulation, comprising contacting a cell of a subject with the RNAi oligonucleotide of claim 1, or a pharmaceutically acceptable salt thereof.

68. The method of claim 67, wherein the cell is contacted for a time sufficient to obtain degradation of an mRNA transcript of C3, wherein:
(a) the expression of C3 in the cell is reduced; and/or
(b) the translation of C3 mRNA in the cell is reduced; and/or
(c) the level and/or activity of C3 in the cell is reduced.

69-75. (canceled)

76. The method of claim 67, wherein the subject is identified as having a disease, disorder, or condition mediated by complement pathway activation or dysregulation.

77-81. (canceled)

82. A method for reducing C3 expression in a cell, a population of cells, or a subject, the method comprising the step of:
i) contacting the cell or the population of cells with the RNAi oligonucleotide of claim 1, or a pharmaceutically acceptable salt thereof, or the pharmaceutical composition of claim 66; or
ii) administering to the subject the RNAi oligonucleotide of claim 1, or a pharmaceutically acceptable salt thereof, or the pharmaceutical composition of claim 66, wherein optionally reducing C3 expression comprises reducing an amount or level of C3 mRNA, level of C3 protein, or both by about 10% to 100% relative to the level of C3 mRNA, level of C3 protein, or both in the cell of a subject that is not administered the RNAi oligonucleotide of claim 1, or a pharmaceutically acceptable salt thereof.

83-87. (canceled)

88. A kit comprising the RNAi oligonucleotide of claim 1, or a pharmaceutically acceptable salt thereof.

89-91. (canceled)

92. The RNA oligonucleotide of claim 1, wherein the RNA oligonucleotide comprises a pharmaceutically acceptable salt, wherein the pharmaceutically acceptable salt is a sodium salt.

93. (canceled)


SEQUENCE LISTING (67 sequences)

SEQ ID NO: 1 - RNA, 36 nt, Artificial Sequence
  Synthetic Construct
aucaacucaccuguaauaaagcagccgaaaggcugc

SEQ ID NO: 2 - RNA, 20 nt, Artificial Sequence
  Synthetic Construct
aucaacucaccuguaauaaa

SEQ ID NO: 3 - RNA, 22 nt, Artificial Sequence
  Synthetic Construct
uuuauuacaggugaguugaugg

SEQ ID NO: 4 - RNA, 36 nt, Artificial Sequence
  Synthetic Construct
agaaauucuacuacaucuaagcagccgaaaggcugc

SEQ ID NO: 5 - RNA, 20 nt, Artificial Sequence
  Synthetic Construct
agaaauucuacuacaucuaa

SEQ ID NO: 6 - RNA, 22 nt, Artificial Sequence
  Synthetic Construct
uuagauguaguagaauuucugg

SEQ ID NO: 7 - RNA, 16 nt, Artificial Sequence
  Synthetic Construct
gcagccgaaaggcugc

SEQ ID NO: 8 - RNA, 4 nt, Artificial Sequence
  Synthetic Construct
gaaa

SEQ ID NO: 9 - RNA, 58 nt, Artificial Sequence
  Synthetic Construct
aucaacucaccuguaauaaagcagccgaaaggcugcuuuauuacaggugaguugaugg

SEQ ID NO: 10 - RNA, 58 nt, Artificial Sequence
  Synthetic Construct
agaaauucuacuacaucuaagcagccgaaaggcugcuuagauguaguagaauuucugg

SEQ ID NO: 11 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 12 - DNA, 5231 bp, Homo sapiens
actcctccccatcctctccctctgtccctctgtccctctgaccctgcactgtcccagcaccatgggacccacctcaggtcccagcctgctgctcctgctactaacccacctccccctggctctggggagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacccccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgcccgccgacgccgttccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaacctggatgaggacatcattgcagaagagaacatcgtttcccgaagtgagttcccagagagctggctgtggaacgttgaggacttgaaagagccaccgaaaaatggaatctctacgaagctcatgaatatatttttgaaagactccatcaccacgtgggagattctggctgtgagcatgtcggacaagaaagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgctgtctaccatcatttcatcagtgacggtgtcaggaagtccctgaaggtcgtgccggaaggaatcagaatgaacaaaactgtggctgttcgcaccctggatccagaacgcctgggccgtgaaggagtgcagaaagaggacatcccacctgcagacctcagtgaccaagtcccggacaccgagtctgagaccagaattctcctgcaagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcccgaggaggacgaatgccaagacgaagagaaccagaaacaatgccaggacctcggcgccttcaccgagagcatggttgtctttgggtgccccaactgaccacacccccattcccccactccagataaagcttcagttatatctcacgtgtctggagttctttgccaagagggagaggctgaaatccccagccgcctcacctgcagctcagctccatcctacttgaaacctcacctgttcccaccgcattttctcctggcgttcgcctgctagtgtg

SEQ ID NO: 13 - DNA, 20 bp, Artificial Sequence
  Synthetic Construct
atcaactcacctgtaataaa

SEQ ID NO: 14 - DNA, 19 bp, Artificial Sequence
  Synthetic Construct
agaaattctactacatcta

SEQ ID NO: 15 - RNA, 37 nt, Artificial Sequence
  Synthetic Construct
aggaaugagaaucaacaaaaagcagccgaaaggcugc

SEQ ID NO: 16 - RNA, 21 nt, Artificial Sequence
  Synthetic Construct
uuuuguugauucucauuccug

SEQ ID NO: 17 - RNA, 36 nt, Artificial Sequence
  Synthetic Construct
cagcuaaaagacuuugacuagcagccgaaaggcugc

SEQ ID NO: 18 - RNA, 22 nt, Artificial Sequence
  Synthetic Construct
uagucaaagucuuuuagcuggg

SEQ ID NO: 19 - RNA, 36 nt, Artificial Sequence
  Synthetic Construct
gcuaaaagacuuugacuuuagcagccgaaaggcugc

SEQ ID NO: 20 - RNA, 22 nt, Artificial Sequence
  Synthetic Construct
uaaagucaaagucuuuuagcgg

SEQ ID NO: 21 - RNA, 36 nt, Artificial Sequence
  Synthetic Construct
cucaaugaacagagauacuagcagccgaaaggcugc

SEQ ID NO: 22 - RNA, 22 nt, Artificial Sequence
  Synthetic Construct
uaguaucucuguucauugaggg

SEQ ID NO: 23 - RNA, 36 nt, Artificial Sequence
  Synthetic Construct
ucaacucaccuguaauaaaagcagccgaaaggcugc

SEQ ID NO: 24 - RNA, 22 nt, Artificial Sequence
  Synthetic Construct
uuuuauuacaggugaguugagg

SEQ ID NO: 25 - RNA, 36 nt, Artificial Sequence
  Synthetic Construct
ugaggagaauugcuucauaagcagccgaaaggcugc

SEQ ID NO: 26 - RNA, 22 nt, Artificial Sequence
  Synthetic Construct
uuaugaagcaauucuccucagg

SEQ ID NO: 27 - RNA, 36 nt, Artificial Sequence
  Synthetic Construct
ggagaauugcuucauacaaagcagccgaaaggcugc

SEQ ID NO: 28 - RNA, 22 nt, Artificial Sequence
  Synthetic Construct
uuuguaugaagcaauucuccgg

SEQ ID NO: 29 - RNA, 36 nt, Artificial Sequence
  Synthetic Construct
agauaaagcuucaguuauaagcagccgaaaggcugc

SEQ ID NO: 30 - RNA, 22 nt, Artificial Sequence
  Synthetic Construct
uuauaacugaagcuuuaucugg

SEQ ID NO: 31 - DNA, 20 bp, Artificial Sequence
  Synthetic Construct
aggaatgagaatcaacaaaa

SEQ ID NO: 32 - DNA, 5139 bp, Mus musculus
agagaggagagccatataaagagccagcggctacagccccagctcgcctctgcccacccctgccccttaccccttcattccttccacctttttccttcactatgggaccagcttcagggtcccagctactagtgctactgctgctgttggccagctccccattagctctggggatccccatgtattccatcattactcccaatgtcctacggctggagagcgaagagaccatcgtactggaggcccacgatgctcagggtgacatcccagtcacagtcactgtgcaagacttcctaaagaggcaagtgctgaccagtgagaagacagtgttgacaggagccagtggacatctgagaagcgtctccatcaagattccagccagtaaggaattcaactcagataaggaggggcacaagtacgtgacagtggtggcaaacttcggggaaacggtggtggagaaagcagtgatggtaagcttccagagtgggtacctcttcatccagacagacaagaccatctacacccctggctccactgtcttatatcggatcttcactgtggacaacaacctactgcccgtgggcaagacagtcgtcatcctcattgagacccccgatggcattcctgtcaagagagacattctgtcttccaacaaccaacacggcatcttgcctttgtcttggaacattcctgaactggtcaacatggggcagtggaagatccgagccttttacgaacatgcgccgaagcagatcttctccgcagagtttgaggtgaaggaatacgtgctgcccagttttgaggtccgggtggagcccacagagacattttattacatcgatgacccaaatggcctggaagtttccatcatagccaagttcctgtacgggaaaaacgtggacgggacagccttcgtgatttttggggtccaggatggcgataagaagatttctctggcccactccctcacgcgcgtagtgattgaggatggtgtgggggatgcagtgctgacccggaaggtgctgatggagggggtacggccttccaacgccgacgccctggtggggaagtccctgtatgtctccgtcactgtcatcctgcactcaggtagtgacatggtagaggcagagcgcagtgggatcccgattgtcacttccccgtaccagatccacttcaccaagacacccaaattcttcaagccagccatgccctttgacctcatggtgttcgtgaccaaccccgatggctctccggccagcaaagtgctggtggtcactcagggatctaatgcaaaggctctcacccaagatgatggcgtggccaagctaagcatcaacacacccaacagccgccaacccctgaccatcacagtccgcaccaagaaggacactctcccagaatcacggcaggccaccaagacaatggaggcccatccctacagcactatgcacaactccaacaactacctacacttgtcagtgtcacgaatggagctcaagccgggggacaacctcaatgtcaacttccacctgcgcacagacccaggccatgaggccaagatccgatactacacctacctggttatgaacaaggggaagctcctgaaggcaggccgccaggttcgggagcctggccaggacctggtggtcttgtccctgcccatcactccagagtttattccttcatttcgcctggtggcttactacaccctgattggagctagtggccagagggaggtggtggctgactctgtgtgggtggatgtgaaggattcctgtattggcacgctggtggtgaagggtgacccaagagataaccatctcgcacctgggcaacaaacgacactcaggattgaaggaaaccagggggcccgagtggggctagtggctgtggacaagggagtgtttgtgctgaacaagaagaacaaactcacacagagcaagatctgggatgtggtagagaaggcagacattggctgcaccccaggcagtgggaagaactatgctggtgtcttcatggatgcaggcctggccttcaagacaagccaaggactgcagactgaacagagagcagatcttgagtgcaccaagccagcagcccgccgccgtcgctcagtacagttgatggaaagaaggatggacaaagctggtcagtacactgacaagggtcttcggaagtgttgtgaggatggtatgcgggatatccctatgagatacagctgccagcgccgggcacgcctcatcacccagggcgagaactgcataaaggccttcatagactgctgcaaccacatcaccaagctgcgtgaacaacacagaagagaccacgtgctgggcctggccaggagtgaattggaggaagacataattccagaagaagatattatctctagaagccacttcccacagagctggttgtggaccatagaagagttgaaagaaccagagaaaaatggaatctctacgaaggtcatgaacatctttctcaaagattccatcaccacctgggagattctggcagtgagcttgtcagacaagaaagggatctgtgtggcagacccctatgagatcagagtgatgcaggacttcttcattgacctgcggctgccctactctgtagtgcgcaacgaacaggtggagatcagagctgtgctcttcaactaccgtgaacaggaggaacttaaggtgagggtggaactgttgcataatccagccttctgcagcatggccaccgccaagaatcgctacttccagaccatcaaaatccctcccaagtcctcggtggctgtaccgtatgtcattgtccccttgaagatcggccaacaagaggtggaggtcaaggctgctgtcttcaatcacttcatcagtgatggtgtcaagaagacactgaaggtcgtgccagaaggaatgagaatcaacaaaactgtggccatccatacactggacccagagaagctcggtcaagggggagtgcagaaggtggatgtgcctgccgcagaccttagcgaccaagtgccagacacagactctgagaccagaattatcctgcaagggagcccggtggttcagatggctgaagatgctgtggacggggagcggctgaaacacctgatcgtgacccccgcaggctgtggggaacagaacatgattggcatgacaccaacagtcattgcggtacactacctggaccagaccgaacagtgggagaagttcggcatagagaagaggcaagaggccctggagctcatcaagaaagggtacacccagcagctggccttcaaacagcccagctctgcctatgctgccttcaacaaccggccccccagcacctggctgacagcctacgtggtcaaggtcttctctctagctgccaacctcatcgccatcgactctcacgtcctgtgtggggctgttaaatggttgattctggagaaacagaagccggatggtgtctttcaggaggatgggcccgtgattcaccaagaaatgattggtggcttccggaacgccaaggaggcagatgtgtcactcacagccttcgtcctcatcgcactgcaggaagccagggacatctgtgaggggcaggtcaatagccttcctgggagcatcaacaaggcaggggagtatattgaagccagttacatgaacctgcagagaccatacacagtggccattgctgggtatgccctggccctgatgaacaaactggaggaaccttacctcggcaagtttctgaacacagccaaagatcggaaccgctgggaggagcctgaccagcagctctacaacgtagaggccacatcctacgccctcctggccctgctgctgctgaaagactttgactctgtgccccctgtagtgcgctggctcaatgagcaaagatactacggaggcggctatggctccacccaggctaccttcatggtattccaagccttggcccaatatcaaacagatgtccctgaccataaggacttgaacatggatgtgtccttccacctccccagccgtagctctgcaaccacgtttcgcctgctctgggaaaatggcaacctcctgcgatcggaagagaccaagcaaaatgaggccttctctctaacagccaaaggaaaaggccgaggcacattgtcggtggtggcagtgtatcatgccaaactcaaaagcaaagtcacctgcaagaagtttgacctcagggtcagcataagaccagcccctgagacagccaagaagcccgaggaagccaagaataccatgttccttgaaatctgcaccaagtacttgggagatgtggacgccactatgtccatcctggacatctccatgatgactggctttgctccagacacaaaggacctggaactgctggcctctggagtagatagatacatctccaagtacgagatgaacaaagccttctccaacaagaacaccctcatcatctacctagaaaagatttcacacaccgaagaagactgcctgaccttcaaagttcaccagtactttaatgtgggacttatccagcccgggtcggtcaaggtctactcctattacaacctcgaggaatcatgcacccggttctatcatccagagaaggacgatgggatgctcagcaagctgtgccacagtgaaatgtgccggtgtgctgaagagaactgcttcatgcaacagtcacaggagaagatcaacctgaatgtccggctagacaaggcttgtgagcccggagtcgactatgtgtacaagaccgagctaaccaacatagagctgttggatgattttgatgagtacaccatgaccatccagcaggtcatcaagtcaggctcagatgaggtgcaggcagggcagcaacgcaagttcatcagccacatcaagtgcagaaacgccctgaagctgcagaaagggaagaagtacctcatgtggggcctctcctctgacctctggggagaaaagcccaacaccagctacatcattgggaaggacacgtgggtggagcactggcctgaggcagaagaatgccaggatcagaagtaccagaaacagtgcgaagaacttggggcattcacagaatctatggtggtttatggttgtcccaactgactacagcccagccctctaataaagcttcagttgtatttcacccatc

SEQ ID NO: 33 - DNA, 36 bp, Artificial Sequence
  Synthetic Construct
atcaactcacctgtaataaagcagccgaaaggctgc

SEQ ID NO: 34 - DNA, 22 bp, Artificial Sequence
  Synthetic Construct
tttattacaggtgagttgatgg

SEQ ID NO: 35 - DNA, 36 bp, Artificial Sequence
  Synthetic Construct
agaaattctactacatctaagcagccgaaaggctgc

SEQ ID NO: 36 - DNA, 22 bp, Artificial Sequence
  Synthetic Construct
ttagatgtagtagaatttctgg

SEQ ID NO: 37 - RNA, 36 nt, Artificial Sequence; identical to SEQ ID NO: 1
  Synthetic Construct
  modified_base (1)..(1): 2′-O-methyladenosine phosphorothioate
  modified_base (2)..(2): um
  modified_base (3)..(3): cm
  modified_base (4)..(5): 2′-O-methyladenosine
  modified_base (6)..(6): cm
  modified_base (7)..(7): um
  modified_base (8)..(8): 2′-fluorocytidine
  modified_base (9)..(9): 2′-fluoroadenosine
  modified_base (10)..(11): 2′-fluorocytidine
  modified_base (12)..(12): um
  modified_base (13)..(13): gm
  modified_base (14)..(14): um
  modified_base (15)..(16): 2′-O-methyladenosine
  modified_base (17)..(17): um
  modified_base (18)..(20): 2′-O-methyladenosine
  modified_base (21)..(21): gm
  modified_base (22)..(22): cm
  modified_base (23)..(23): 2′-O-methyladenosine
  modified_base (24)..(24): gm
  modified_base (25)..(26): cm
  modified_base (27)..(27): gm
  modified_base (28)..(30): 2′aminodiethoxymethanol-Adenine-GalNAc
  modified_base (31)..(32): gm
  modified_base (33)..(33): cm
  modified_base (34)..(34): um
  modified_base (35)..(35): gm
  modified_base (36)..(36): cm

SEQ ID NO: 38 - RNA, 22 nt, Artificial Sequence
  Synthetic Construct
  modified_base (1)..(1): 5′Methoxy, Phosphonate-4′oxy-2′-O-methyluridine phosporothioate
  modified_base (2)..(2): 2′-fluorouridine phosphorothioate
  modified_base (3)..(3): 2′-fluorouridine
  modified_base (4)..(4): 2′-fluoroadenosine
  modified_base (5)..(5): 2′-fluorouridine
  modified_base (6)..(6): um
  modified_base (7)..(7): 2′-fluoroadenosine
  modified_base (8)..(8): cm
  modified_base (9)..(9): 2′-O-methyladenosine
  modified_base (10)..(10): 2′-fluoroguanosine
  modified_base (11)..(11): gm
  modified_base (12)..(12): um
  modified_base (13)..(13): gm
  modified_base (14)..(14): 2′-fluoroadenosine
  modified_base (15)..(15): gm
  modified_base (16)..(17): um
  modified_base (18)..(18): gm
  modified_base (19)..(19): 2′-O-methyladenosine
  modified_base (20)..(20): 2′-O-methyluridine phosphorothioate
  modified_base (21)..(21): 2′-O-methylguanosine phosphorothioate
  modified_base (22)..(22): gm
uuuauuacaggugaguugaugg

SEQ ID NO: 39 - RNA, 36 nt, Artificial Sequence; identical to SEQ ID NO: 4
  Synthetic Construct
  modified_base (1)..(1): 2′-O-methyladenosine phosphorothioate
  modified_base (2)..(2): gm
  modified_base (3)..(3): 2′-fluoroadenosine
  modified_base (4)..(5): 2′-O-methyladenosine
  modified_base (6)..(7): um
  modified_base (8)..(8): 2′-fluorocytidine
  modified_base (9)..(9): 2′-fluorouridine
  modified_base (10)..(10): 2′-fluoroadenosine
  modified_base (11)..(11): cm
  modified_base (12)..(12): 2′-fluorouridine
  modified_base (13)..(13): 2′-fluoroadenosine
  modified_base (14)..(14): cm
  modified_base (15)..(15): 2′-O-methyladenosine
  modified_base (16)..(16): um
  modified_base (17)..(17): 2′-fluorocytidine
  modified_base (18)..(18): um
  modified_base (19)..(20): 2′-O-methyladenosine
  modified_base (21)..(21): gm
  modified_base (22)..(22): cm
  modified_base (23)..(23): 2′-O-methyladenosine
  modified_base (24)..(24): gm
  modified_base (25)..(26): cm
  modified_base (27)..(27): gm
  modified_base (28)..(30): 2′aminodiethoxymethanol-Adenine-GalNAc
  modified_base (31)..(32): gm
  modified_base (33)..(33): cm
  modified_base (34)..(34): um
  modified_base (35)..(35): gm
  modified_base (36)..(36): cm

SEQ ID NO: 40 - RNA, 22 nt, Artificial Sequence
  Synthetic Construct
  modified_base (1)..(1): 5′Methoxy, Phosphonate-4′oxy-2′-O-methyluridine phosporothioate
  modified_base (2)..(2): 2′-fluorouridine phosphorothioate
  modified_base (3)..(3): 2′-fluoroadenosine
  modified_base (4)..(4): 2′-fluoroguanosine
  modified_base (5)..(5): 2′-fluoroadenosine
  modified_base (6)..(6): um
  modified_base (7)..(7): 2′-fluoroguanosine
  modified_base (8)..(8): 2′-fluorouridine
  modified_base (9)..(9): 2′-O-methyladenosine
  modified_base (10)..(10): 2′-fluoroguanosine
  modified_base (11)..(11): um
  modified_base (12)..(12): 2′-fluoroadenosine
  modified_base (13)..(13): gm
  modified_base (14)..(14): 2′-fluoroadenosine
  modified_base (15)..(15): 2′-O-methyladenosine
  modified_base (16)..(16): 2′-fluorouridine
  modified_base (17)..(18): um
  modified_base (19)..(19): 2′-fluorocytidine
  modified_base (20)..(20): 2′-O-methyluridine phosphorothioate
  modified_base (21)..(21): 2′-O-methylguanosine phosphorothioate
  modified_base (22)..(22): gm
uuagauguaguagaauuucugg

SEQ ID NO: 41 - RNA, 36 nt, Artificial Sequence; identical to SEQ ID NO: 17
  Synthetic Construct
  modified_base (1)..(1): 2′-O-methylcytidine phosphorothioate
  modified_base (2)..(2): 2′-O-methyladenosine
  modified_base (3)..(3): gm
  modified_base (4)..(4): cm
  modified_base (5)..(5): um
  modified_base (6)..(7): 2′-O-methyladenosine
  modified_base (8)..(9): 2′-fluoroadenosine
  modified_base (10)..(10): 2′-fluoroguanosine
  modified_base (11)..(11): 2′-fluoroadenosine
  modified_base (12)..(12): cm
  modified_base (13)..(15): um
  modified_base (16)..(16): gm
  modified_base (17)..(17): 2′-O-methyladenosine
  modified_base (18)..(18): cm
  modified_base (19)..(19): um
  modified_base (20)..(20): 2′-O-methyladenosine
  modified_base (21)..(21): gm
  modified_base (22)..(22): cm
  modified_base (23)..(23): 2′-O-methyladenosine
  modified_base (24)..(24): gm
  modified_base (25)..(26): cm
  modified_base (27)..(27): gm
  modified_base (28)..(30): 2′aminodiethoxymethanol-Adenine-GalNAc
  modified_base (31)..(32): gm
  modified_base (33)..(33): cm
  modified_base (34)..(34): um
  modified_base (35)..(35): gm
  modified_base (36)..(36): cm

SEQ ID NO: 42 - RNA, 22 nt, Artificial Sequence
  Synthetic Construct
  modified_base (1)..(1): 5′Methoxy, Phosphonate-4′oxy-2′-O-methyluridine phosporothioate
  modified_base (2)..(2): 2′-fluoroadenosine phosphorothioate
  modified_base (3)..(3): 2′-fluoroguanosine
  modified_base (4)..(4): 2′-fluorouridine
  modified_base (5)..(5): 2′-fluorocytidine
  modified_base (6)..(6): 2′-O-methyladenosine
  modified_base (7)..(7): 2′-fluoroadenosine
  modified_base (8)..(8): 2′-O-methyladenosine
  modified_base (9)..(9): gm
  modified_base (10)..(10): 2′-fluorouridine
  modified_base (11)..(11): cm
  modified_base (12)..(13): um
  modified_base (14)..(14): 2′-fluorouridine
  modified_base (15)..(15): um
  modified_base (16)..(16): 2′-O-methyladenosine
  modified_base (17)..(17): gm
  modified_base (18)..(18): cm
  modified_base (19)..(19): um
  modified_base (20)..(21): 2′-O-methylguanosine phosphorothioat
  modified_base (22)..(22): gm
uagucaaagucuuuuagcuggg

SEQ ID NO: 43 - RNA, 36 nt, Artificial Sequence; identical to SEQ ID NO: 19
  Synthetic Construct
  modified_base (1)..(1): 2′-O-methylguanosine phosphorothioate
  modified_base (2)..(2): cm
  modified_base (3)..(3): um
  modified_base (4)..(7): 2′-O-methyladenosine
  modified_base (8)..(8): 2′-fluoroguanosine
  modified_base (9)..(9): 2′-fluoroadenosine
  modified_base (10)..(10): 2′-fluorocytidine
  modified_base (11)..(11): 2′-fluorouridine
  modified_base (12)..(13): um
  modified_base (14)..(14): gm
  modified_base (15)..(15): 2′-O-methyladenosine
  modified_base (16)..(16): cm
  modified_base (17)..(19): um
  modified_base (20)..(20): 2′-O-methyladenosine
  modified_base (21)..(21): gm
  modified_base (22)..(22): cm
  modified_base (23)..(23): 2′-O-methyladenosine
  modified_base (24)..(24): gm
  modified_base (25)..(26): cm
  modified_base (27)..(27): gm
  modified_base (28)..(30): 2′aminodiethoxymethanol-Adenine-GalNAc
  modified_base (31)..(32): gm
  modified_base (33)..(33): cm
  modified_base (34)..(34): um
  modified_base (35)..(35): gm
  modified_base (36)..(36): cm

SEQ ID NO: 44 - RNA, 22 nt, Artificial Sequence
  Synthetic Construct
  modified_base (1)..(1): 5′Methoxy, Phosphonate-4′oxy-2′-O-methyluridine phosporothioate
  modified_base (2)..(2): 2′-fluoroadenosine phosphorothioate
  modified_base (3)..(4): 2′-fluoroadenosine
  modified_base (5)..(5): 2′-fluoroguanosine
  modified_base (6)..(6): um
  modified_base (7)..(7): 2′-fluorocytidine
  modified_base (8)..(9): 2′-O-methyladenosine
  modified_base (10)..(10): 2′-fluoroadenosine
  modified_base (11)..(11): gm
  modified_base (12)..(12): um
  modified_base (13)..(13): cm
  modified_base (14)..(14): 2′-fluorouridine
  modified_base (15)..(17): um
  modified_base (18)..(18): 2′-O-methyladenosine
  modified_base (19)..(19): gm
  modified_base (20)..(20): 2′-O-methylcytidine phosphorothioate
  modified_base (21)..(21): 2′-O-methylguanosine phosphorothioate
  modified_base (22)..(22): gm
uaaagucaaagucuuuuagcgg

SEQ ID NO: 45 - RNA, 36 nt, Artificial Sequence; identical to SEQ ID NO: 21
  Synthetic Construct
  modified_base (1)..(1): 2′-O-methylcytidine phosphorothioate
  modified_base (2)..(2): um
  modified_base (3)..(3): cm
  modified_base (4)..(5): 2′-O-methyladenosine
  modified_base (6)..(6): um
  modified_base (7)..(7): gm
  modified_base (8)..(9): 2′-fluoroadenosine
  modified_base (10)..(10): 2′-fluorocytidine
  modified_base (11)..(11): 2′-fluoroadenosine
  modified_base (12)..(12): gm
  modified_base (13)..(13): 2′-O-methyladenosine
  modified_base (14)..(14): gm
  modified_base (15)..(15): 2′-O-methyladenosine
  modified_base (16)..(16): um
  modified_base (17)..(17): 2′-O-methyladenosine
  modified_base (18)..(18): cm
  modified_base (19)..(19): um
  modified_base (20)..(20): 2′-O-methyladenosine
  modified_base (21)..(21): gm
  modified_base (22)..(22): cm
  modified_base (23)..(23): 2′-O-methyladenosine
  modified_base (24)..(24): gm
  modified_base (25)..(26): cm
  modified_base (27)..(27): gm
  modified_base (28)..(30): 2′aminodiethoxymethanol-Adenine-GalNAc
  modified_base (31)..(32): gm
  modified_base (33)..(33): cm
  modified_base (34)..(34): um
  modified_base (35)..(35): gm
  modified_base (36)..(36): cm

SEQ ID NO: 46 - RNA, 22 nt, Artificial Sequence
  Synthetic Construct
  modified_base (1)..(1): 5′Methoxy, Phosphonate-4′oxy-2′-O-methyluridine phosporothioate
  modified_base (2)..(2): 2′-fluoroadenosine phosphorothioate
  modified_base (3)..(3): 2′-fluoroguanosine
  modified_base (4)..(4): 2′-fluorouridine
  modified_base (5)..(5): 2′-fluoroadenosine
  modified_base (6)..(6): um
  modified_base (7)..(7): 2′-fluorocytidine
  modified_base (8)..(8): um
  modified_base (9)..(9): cm
  modified_base (10)..(10): 2′-fluorouridine
  modified_base (11)..(11): gm
  modified_base (12)..(13): um
  modified_base (14)..(14): 2′-fluorocytidine
  modified_base (15)..(15): 2′-O-methyladenosine
  modified_base (16)..(17): um
  modified_base (18)..(18): gm
  modified_base (19)..(19): 2′-O-methyladenosine
  modified_base (20)..(21): 2′-O-methylguanosine phosphorothioate
  modified_base (22)..(22): gm
uaguaucucuguucauugaggg

SEQ ID NO: 47 - RNA, 36 nt, Artificial Sequence; identical to SEQ ID NO: 23
  Synthetic Construct
  modified_base (1)..(1): 2′-O-methyluridine phosphorothioate
  modified_base (2)..(2): cm
  modified_base (3)..(4): 2′-O-methyladenosine
  modified_base (5)..(5): cm
  modified_base (6)..(6): um
  modified_base (7)..(7): cm
  modified_base (8)..(8): 2′-fluoroadenosine
  modified_base (9)..(10): 2′-fluorocytidine
  modified_base (11)..(11): 2′-fluorouridine
  modified_base (12)..(12): gm
  modified_base (13)..(13): um
  modified_base (14)..(15): 2′-O-methyladenosine
  modified_base (16)..(16): um
  modified_base (17)..(20): 2′-O-methyladenosine
  modified_base (21)..(21): gm
  modified_base (22)..(22): cm
  modified_base (23)..(23): 2′-O-methyladenosine
  modified_base (24)..(24): gm
  modified_base (25)..(26): cm
  modified_base (27)..(27): gm
  modified_base (28)..(30): 2′aminodiethoxymethanol-Adenine-GalNAc
  modified_base (31)..(32): gm
  modified_base (33)..(33): cm
  modified_base (34)..(34): um
  modified_base (35)..(35): gm
  modified_base (36)..(36): cm

SEQ ID NO: 48 - RNA, 22 nt, Artificial Sequence
  Synthetic Construct
  modified_base (1)..(1): 5′Methoxy, Phosphonate-4′oxy-2′-O-methyluridine phosporothioate
  modified_base (2)..(2): 2′-fluorouridine phosphorothioate
  modified_base (3)..(4): 2′-fluorouridine
  modified_base (5)..(5): 2′-fluoroadenosine
  modified_base (6)..(6): um
  modified_base (7)..(7): 2′-fluorouridine
  modified_base (8)..(8): 2′-O-methyladenosine
  modified_base (9)..(9): cm
  modified_base (10)..(10): 2′-fluoroadenosine
  modified_base (11)..(12): gm
  modified_base (13)..(13): um
  modified_base (14)..(14): 2′-fluoroguanosine
  modified_base (15)..(15): 2′-O-methyladenosine
  modified_base (16)..(16): gm
  modified_base (17)..(18): um
  modified_base (19)..(19): gm
  modified_base (20)..(20): 2′-O-methyladenosine phosphorothioate
  modified_base (21)..(21): 2′-O-methylguanosine phosphorothioate
  modified_base (22)..(22): gm
uuuuauuacaggugaguugagg

SEQ ID NO: 49 - RNA, 36 nt, Artificial Sequence; identical to SEQ ID NO: 25
  Synthetic Construct
  modified_base (1)..(1): 2′-O-methyluridine phosphorothioate
  modified_base (2)..(2): gm
  modified_base (3)..(3): 2′-O-methyladenosine
  modified_base (4)..(5): gm
  modified_base (6)..(6): 2′-O-methyladenosine
  modified_base (7)..(7): gm
  modified_base (8)..(9): 2′-fluoroadenosine
  modified_base (10)..(11): 2′-fluorouridine
  modified_base (12)..(12): gm
  modified_base (13)..(13): cm
  modified_base (14)..(15): um
  modified_base (16)..(16): cm
  modified_base (17)..(17): 2′-O-methyladenosine
  modified_base (18)..(18): um
  modified_base (19)..(20): 2′-O-methyladenosine
  modified_base (21)..(21): gm
  modified_base (22)..(22): cm
  modified_base (23)..(23): 2′-O-methyladenosine
  modified_base (24)..(24): gm
  modified_base (25)..(26): cm
  modified_base (27)..(27): gm
  modified_base (28)..(30): 2′aminodiethoxymethanol-Adenine-GalNAc
  modified_base (31)..(32): gm
  modified_base (33)..(33): cm
  modified_base (34)..(34): um
  modified_base (35)..(35): gm
  modified_base (36)..(36): cm

SEQ ID NO: 50 - RNA, 22 nt, Artificial Sequence
  Synthetic Construct
  modified_base (1)..(1): 5′Methoxy, Phosphonate-4′oxy-2′-O-methyluridine phosporothioate
  modified_base (2)..(2): 2′-fluorouridine phosphorothioate
  modified_base (3)..(3): 2′-fluoroadenosine
  modified_base (4)..(4): 2′-fluorouridine
  modified_base (5)..(5): 2′-fluoroguanosine
  modified_base (6)..(6): 2′-O-methyladenosine
  modified_base (7)..(7): 2′-fluoroadenosine
  modified_base (8)..(8): gm
  modified_base (9)..(9): cm
  modified_base (10)..(10): 2′-fluoroadenosine
  modified_base (11)..(11): 2′-O-methyladenosine
  modified_base (12)..(13): um
  modified_base (14)..(14): 2′-fluorocytidine
  modified_base (15)..(15): um
  modified_base (16)..(17): cm
  modified_base (18)..(18): um
  modified_base (19)..(19): cm
  modified_base (20)..(20): 2′-O-methyladenosine phosphorothioate
  modified_base (21)..(21): 2′-O-methylguanosine phosphorothioate
  modified_base (22)..(22): gm
uuaugaagcaauucuccucagg

SEQ ID NO: 51 - RNA, 36 nt, Artificial Sequence; identical to SEQ ID NO: 27
  Synthetic Construct
  modified_base (1)..(1): 2′-O-methylguanosine phosphorothioate
  modified_base (2)..(2): gm
  modified_base (3)..(3): 2′-O-methyladenosine
  modified_base (4)..(4): gm
  modified_base (5)..(6): 2′-O-methyladenosine
  modified_base (7)..(7): um
  modified_base (8)..(8): 2′-fluorouridine
  modified_base (9)..(9): 2′-fluoroguanosine
  modified_base (10)..(10): 2′-fluorocytidine
  modified_base (11)..(11): 2′-fluorouridine
  modified_base (12)..(12): um
  modified_base (13)..(13): cm
  modified_base (14)..(14): 2′-O-methyladenosine
  modified_base (15)..(15): um
  modified_base (16)..(16): 2′-O-methyladenosine
  modified_base (17)..(17): cm
  modified_base (18)..(20): 2′-O-methyladenosine
  modified_base (21)..(21): gm
  modified_base (22)..(22): cm
  modified_base (23)..(23): 2′-O-methyladenosine
  modified_base (24)..(24): gm
  modified_base (25)..(26): cm
  modified_base (27)..(27): gm
  modified_base (28)..(30): 2′aminodiethoxymethanol-Adenine-GalNAc
  modified_base (31)..(32): gm
  modified_base (33)..(33): cm
  modified_base (34)..(34): um
  modified_base (35)..(35): gm
  modified_base (36)..(36): cm

SEQ ID NO: 52 - RNA, 22 nt, Artificial Sequence
  Synthetic Construct
  modified_base (1)..(1): 5′Methoxy, Phosphonate-4′oxy-2′-O-methyluridine phosporothioate
  modified_base (2)..(2): 2′-fluorouridine phosphorothioate
  modified_base (3)..(3): 2′-fluorouridine
  modified_base (4)..(4): 2′-fluoroguanosine
  modified_base (5)..(5): 2′-fluorouridine
  modified_base (6)..(6): 2′-O-methyladenosine
  modified_base (7)..(7): 2′-fluorouridine
  modified_base (8)..(8): gm
  modified_base (9)..(9): 2′-O-methyladenosine
  modified_base (10)..(10): 2′-fluoroadenosine
  modified_base (11)..(11): gm
  modified_base (12)..(12): cm
  modified_base (13)..(13): 2′-O-methyladenosine
  modified_base (14)..(14): 2′-fluoroadenosine
  modified_base (15)..(16): um
  modified_base (17)..(17): cm
  modified_base (18)..(18): um
  modified_base (19)..(19): cm
  modified_base (20)..(20): 2′-O-methylcytidine phosphorothioate
  modified_base (21)..(21): 2′-O-methylguanosine phosphorothioate
  modified_base (22)..(22): gm
uuuguaugaagcaauucuccgg

SEQ ID NO: 53 - RNA, 36 nt, Artificial Sequence; identical to SEQ ID NO: 29
  Synthetic Construct
  modified_base (1)..(1): 2′-O-methyladenosine phosphorothioate
  modified_base (2)..(2): gm
  modified_base (3)..(3): 2′-O-methyladenosine
  modified_base (4)..(4): um
  modified_base (5)..(7): 2′-O-methyladenosine
  modified_base (8)..(8): 2′-fluoroguanosine
  modified_base (9)..(9): 2′-fluorocytidine
  modified_base (10)..(11): 2′-fluorouridine
  modified_base (12)..(12): cm
  modified_base (13)..(13): 2′-O-methyladenosine
  modified_base (14)..(14): gm
  modified_base (15)..(16): um
  modified_base (17)..(17): 2′-O-methyladenosine
  modified_base (18)..(18): um
  modified_base (19)..(20): 2′-O-methyladenosine
  modified_base (21)..(21): gm
  modified_base (22)..(22): cm
  modified_base (23)..(23): 2′-O-methyladenosine
  modified_base (24)..(24): gm
  modified_base (25)..(26): cm
  modified_base (27)..(27): gm
  modified_base (28)..(30): 2′aminodiethoxymethanol-Adenine-GalNAc
  modified_base (31)..(32): gm
  modified_base (33)..(33): cm
  modified_base (34)..(34): um
  modified_base (35)..(35): gm
  modified_base (36)..(36): cm

SEQ ID NO: 54 - RNA, 22 nt, Artificial Sequence
  Synthetic Construct
  modified_base (1)..(1): 5′Methoxy, Phosphonate-4′oxy-2′-O-methyluridine phosporothioate
  modified_base (2)..(2): 2′-fluorouridine phosphorothioate
  modified_base (3)..(3): 2′-fluoroadenosine
  modified_base (4)..(4): 2′-fluorouridine
  modified_base (5)..(5): 2′-fluoroadenosine
  modified_base (6)..(6): 2′-O-methyladenosine
  modified_base (7)..(7): 2′-fluorocytidine
  modified_base (8)..(8): um
  modified_base (9)..(9): gm
  modified_base (10)..(10): 2′-fluoroadenosine
  modified_base (11)..(11): 2′-O-methyladenosine
  modified_base (12)..(12): gm
  modified_base (13)..(13): cm
  modified_base (14)..(14): 2′-fluorouridine
  modified_base (15)..(16): um
  modified_base (17)..(17): 2′-O-methyladenosine
  modified_base (18)..(18): um
  modified_base (19)..(19): cm
  modified_base (20)..(20): 2′-O-methyluridine phosphorothioate
  modified_base (21)..(21): 2′-O-methylguanosine phosphorothioate
  modified_base (22)..(22): gm
uuauaacugaagcuuuaucugg

SEQ ID NO: 55 - DNA, 21 bp, Artificial Sequence
  Synthetic Construct
aatgaggccttctctctaaca

SEQ ID NO: 56 - DNA, 20 bp, Artificial Sequence
  Synthetic Construct
acttcttgcaggtgactttg

SEQ ID NO: 57 - DNA, 20 bp, Artificial Sequence
  Synthetic Construct
caaactttgctttccctggt

SEQ ID NO: 58 - DNA, 21 bp, Artificial Sequence
  Synthetic Construct
caacaaagtctggcctgtatc

SEQ ID NO: 59 - DNA, 21 bp, Artificial Sequence
  Synthetic Construct
ccaaactcagcatcaacacac

SEQ ID NO: 60 - DNA, 18 bp, Artificial Sequence
  Synthetic Construct
ctgcatggtcctggtagc

SEQ ID NO: 61 - DNA, 22 bp, Artificial Sequence
  Synthetic Construct
gccaaagatcaactcacctgta

SEQ ID NO: 62 - DNA, 20 bp, Artificial Sequence
  Synthetic Construct
agacatagtggcatcctggt

SEQ ID NO: 63 - DNA, 19 bp, Artificial Sequence
  Synthetic Construct
aatgaactgcaggacgagg

SEQ ID NO: 64 - DNA, 21 bp, Artificial Sequence
  Synthetic Construct
aggtgagatgacaggagatcc

SEQ ID NO: 65 - DNA, 21 bp, Artificial Sequence
  Synthetic Construct
ctttccttggtcaggcagtat

SEQ ID NO: 66 - DNA, 20 bp, Artificial Sequence
  Synthetic Construct
caacacttcgtggagtcctt

SEQ ID NO: 67 - DNA, 5126 bp, Macaca fascicularis
aaagccaactccagcagtcactgctcactcctccccatcctctccctctgtccctctgtccctctgaccctgcactgtcccagcaccatgggactcacctcaggtcccagcctgctgctcctgctactaatccacctccccctggctctggggactcccatgtactctatgatcaccccaaacgtcttgcggctggagagtgaggagaccgtggtgctggaggcccatgacgcgaatggggatgttccggtcactgtcactgtccacgacttcccaggcaaaaaactggtgctgtccagtgagaagaccgtgctgacccctgccaccagccacatgggcagcgtcaccatcaggatcccagccaacaaggagttcaagtcagaaaaggggcacaacaagttcgtgactgtgcaggccaccttcggggcccaagtggtggagaaggtggtactggtcagccttcagagcgggtacctcttcatccagacagacaagaccatctacacccctggctccacagttctctgtcggatcttcaccgtcaaccacaagctgctacccgtgggccggacggtcgtggtcaacattgagaacccggacggcatcccggtcaagcaggactccttgtcttctcagaaccaatttggcatcttgcccttgtcttgggacattccggaactcgtcaacatgggccagtggaagatccgagcctactatgaaaattcgccgcaacaggtcttctccactgagtttgaggtgaaggagtacgtgctgcccagtttcgaggtcatagtggagcctacagagaaattctactacatctataaccagaagggcctggaggtcaccatcaccgccaggttcctctatggaaagaaagtggagggaactgcctttgtcatcttcgggatccaggatggcgagcagaggatttccctgcctgaatccctcaagcgcatccagattgaggatggctcaggagacgccgtgctgagccggaaggtactgctggacggggtgcagaatccccgaccggaagacctagtggggaagtccttgtatgtgtctgtcaccgttatcctgcactcaggcagtgacatggtgcaggcggagcgcagcgggatccccatcgtgacctctccctaccagatccacttcaccaagacgcccaagtacttcaaaccaggaatgccctttgacctcatggtgttcgtgacgaaccccgatggctctccagcctaccgagtccccgtggcagtccagggcgaggacgctgtgcagtctctaacccagggagacggcgtggccaaactcagcatcaacacacaccccagccagaagcccttgagcatcacggtgcgcacgaagaagcgggagctctcggaggcggagcaggctaccaggaccatggaggctcagccctacagcaccgtgggcaactccaacaattacctgcatctctcagtgccacgtgcagagctcagacctggggagaccctcaacgtcaacttcctcctgcgaatggaccgcacccaggaggccaagatccgctactacacctacctgattatgaacaaaggcaagctgttgaaggtgggacgccaggtgcgagagcctggccaggacctggtggtgctgcccctgtccatcaccaccgacttcatcccttccttccgcctggtggcctactacacgctgatcggcgccaacggccagagggaagtggtggccgactccgtgtgggtggacgtcaaggactcttgcgtgggctcgctggtggtaaaaagcggccagtcagaagacaggcagcctttacccgggcagcagatgaccctgaagatagagggtgaccacggggcccgggtgggactggtggctgtggacaagggcgtgtttgtgctgaataagaagaacaagctgacgcagagtaagatctgggacgtggtggagaaggcagacatcggctgcaccccaggcagtgggaaggattacgctggtgtcttctcggatgcaggcctgacctttgcgagcagcagtggccagcagacggcccagagggcagaacttcagtgcccacagccagccgcccgccgacgccgttccgtgcagctcgcggagaagagaatggacaaagttggtcagtaccccaaggagctgcgcaagtgctgcgagcacggtatgcgggagaaccccatgaggttctcatgccagcgccggacccgttacatcaccctggacgaggcgtgcaagaaggccttcctggactgctgcaactacatcaccgagctgcggcggcagcacgcgcgggccagtcacctgggcctggccaggagtaacctggatgaggacatcatcgcagaagagaacatcgtttcccgaagtgagttcccagagagttggctgtggaagattgaagagttgaaagaggcaccgaaaaacggaatctccacgaagctcatgaatatatttttgaaagactccatcaccacgtgggagattctggccgtgagcttgtcagacaagaaagggatctgtgtggcagaccccttcgaggtcacagtaatgcaggacttcttcatcgacctgcggctaccctactctgttgttcgaaacgagcaggtggaaatccgagctgttctctacaattaccggcagaaccaagagctcaaggtgagggtggaactactccacaatccagccttctgcagcctggccaccgccaagaggcgtcaccagcagaccgtaaccatcccccccaagtcctcgctgtccgttccttatgtcatcgtgcccctaaagaccggccagcaggaagtggaagtcaaggctgccgtctaccattttttcatcagtgacggtgtcaggaagtccctgaaggtcgtgccggaaggaatcagaatgaacaaaactgtggctgttcgcacgctggatccagaacgcctgggccaggaaggagtgcagagagaggacgtcccacctgcagacctcagtgaccaagtcccggacaccgagtctgagaccagaattctcctgcaagggaccccggtggcccagatgacagaggatgccatcgatgcggaacggctgaagcacctcatcgtgaccccctcgggctgcggagaacagaacatgatcaccatgacgcccacagtcatcgctgtgcattacctggatgaaacggaacagtgggagaagttcggcccggagaagcggcagggggccttggagctcatcaagaaggggtacacccagcagctggccttcagacaacccagctctgcctttgcggccttcctgaaccgggcacccagcacctggctgaccgcctacgtggtcaaggtcttctctctggctgtcaacctcattgccatcgactcccaggtcctctgcggggctgttaaatggctgatcctggagaagcagaagcccgacggggtcttccaggaggatgcgcccgtgatacatcaagaaatgactggtggattccggaacaccaacgagaaagacatggccctcacggcctttgttctcatctcgctgcaagaggctaaagagatttgcgaggagcaggtcaacagcctgcccggcagcatcactaaagcaggagacttccttgaagccaactacatgaacctacagagatcctacactgtggccatcgctgcctatgccctggcccagatgggcaggctgaagggacctcttctcaacaaatttctgaccacagccaaagataagaaccgctgggaggagcctggtcagcagctctacaatgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggatgtccctgatcacaaggaactgaacctggatgtgtccctccaactgcccagtcgcagctccaagatcatccaccgtatccactgggaatctgccagcctcctgcgatcagaagagaccaaggaaaatgagggtttcacagtcacagctgaaggaaaaggccaaggcaccttgtcggtagtgacaatgtaccatgctaaggccaaaggtcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatactggacatatccatgatgactggcttcgttccagacacagatgacctcaagcagctggcaaacggcgttgacagatacatctccaagtatgagctggacaaagccttctccgataggaacaccctcatcatctacctggacaaggtctcacactctgaggatgactgtatagctttcaaagttcaccaatattttaatgtagagcttatccagcctggtgcagtcaaggtctacgcctattacaacctggcggaaagctgtacccggttctaccacccagaaaaggaggatggaaagctgaacaagctctgtcgtgatgagctgtgccgctgtgctgaggagaattgcttcatacaaaagttggatgacaaagtcaccctggaagaacggctggacaaggcctgtgagccaggagtggactatgtgtacaagacccgactggtcaaggcccagctgtccaatgactttgacgagtacatcatggccattgagcagatcatcaagtcaggctcggatgaggtgcaggttggacaacagcgcacgttcatcagccccatcaagtgcagggaagccctgaagctggaggagaggaaacactacctcatgtggggtctctcctccgatttctggggagagaaacccaatctcagctacatcatcgggaaggacacctgggtggagcactggcccgaggaggacgaatgccaagatgaagagaaccagaaacaatgccaggacctcggcaccttcactgagaacatggttgtctttgggtgccccaactgaccacacccccattcccccactcccaataaagcttcagttatatttca